(19) United States
(12) Patent Application Publication
(10) Pub. No.: US 2024/0139326 A1
(43) Pub. Date: May 2, 2024

(54) METHACRYLAMIDES PROTEIN BINDERS AND USES THEREOF

(71) Applicant: YEDA RESEARCH AND DEVELOPMENT CO. LTD., Rehovot (IL)

(72) Inventors: Nir LONDON, Rehovot (IL); Efrat RESNICK, Rehovot (IL); Rambabu REDDI, Rehovot (IL); Adi ROGEL, Rehovot (IL)

(73) Assignee: YEDA RESEARCH AND DEVELOPMENT CO. LTD., Rehovot (IL)

(21) Appl. No.: 18/258,812

(22) PCT Filed: Dec. 23, 2021
(86) PCT No.: PCT/IL2021/051530

§ 371 Date: Jun. 22, 2023

Related U.S. Application Data
(60) Provisional application No. 63/279,698, filed on Nov. 16, 2021.
(60) Provisional application No. 63/220,517, filed on Jul. 11, 2021.

(30) Foreign Application Priority Data
Dec. 23, 2020 (IL) 279736

(51) Int. Cl. A61K 47/55; C09K 11/07

(52) U.S. Cl. CPC A61K 47/55; C09K 11/07; C09K 2211/1007; C09K 2211/1014; C09K 2211/1022

(57) ABSTRACT

This invention is directed to substituted a methacrylamide compounds as targeted covalent protein binders and uses thereof.

### FIELD OF INTEREST

This invention is directed to substituted a methacrylamide compounds as targeted covalent protein binders and uses thereof.

### BACKGROUND

Selective post-translational modifications (PTMs) of native proteins in cells with chemical probes are a powerful tool to tune and investigate protein function, conformation, structure, cellular signaling, localization, and more. Fluorescent labeling of a protein of interest (POI) is a prominent example that can enable imaging, analysis of the structure, function, dynamics, and localization of a target protein (1,2). Other modifications can control the stability (3), activity (4), and localization (5) of a target protein.

Genetic engineering methods allow the introduction of a fluorescent domain (6), or a chemically reactive domain (7) which enables selective labeling of exogenously expressed proteins.

These approaches, however, typically rely on overexpressed proteins, and the newly introduced domains can be large and perturb the very same process they aim to investigate (8-10). Genetic code expansion enables site-specific incorporation of unnatural amino acids bearing bioorthogonal reactive handles (11-12). The subsequent bio-orthogonal reaction with a suitable complementary reactive functionality allows effective and selective bio-conjugation. This circumvents the introduction of a large domain, but these methods are laborious and require specifically engineered cells (11), limiting their scope.

An alternative to genetic methods is chemical bioconjugation. Several chemical reactions for modifying naturally occurring amino acids while elegantly controlling the selectivity of the probes have been developed for in-vitro protein labeling and allowed the generation of well-defined biotherapeutics and PTM mimics (12-19).

In order to selectively label endogenous proteins even in the crowded environment of live cells, various molecules comprising a target recognition moiety, a reactive functionality, and a probe moiety (or tag) were developed (20-23) In these cases, the protein targeted by traditional affinity labeling often loses its native activity since the recognition moiety permanently occupies its ligand-binding pocket. This may hinder the investigation of protein involvement in relevant cellular processes.

Targeted covalent protein binders or inhibitors are an important class of drugs and chemical probes. However, relatively few electrophiles meet the criteria for successful covalent inhibitor design.

Over the last decade, Hamachi et al have pioneered ligand-directed chemistries which include ligand-directed, -tosyl (LDT)⁴⁹, -acyl imidazole (LDAI)³³, -bromo benzoate (LDBB)⁵⁰, -sulfonyl pyridine⁵¹, and —N-acyl-N-alkyl sulfonamide (LDNASA)³⁵ chemistries. In these bio-conjugation methods, the ligand leaves the active site after forming a covalent bond with nucleophilic residue on the POI⁴⁵. Although these methods enabled prominent applications, and could retain target protein activity^{52,53}, some challenges remain. First, the size of the required activating groups and/or linkers is substantial and precludes the labelling of residues very close to the active site. Second, the nucleophile itself is not rationally selected—it is empirically discovered what residue ends up reacting with the probe, therefore it is hard to assess which target would be amenable to the chemistry. Lastly, some of these chemistries suffer from slow kinetics, low stability in the cellular environment, and structural complexity. Hence, there is a need to develop new ligand-directed chemistries using simple and small reactive groups to reach the desired location and specifically label particular nucleophilic amino acids.

Acrylamides have been widely used as electrophiles for irreversible covalent inhibitors for many proteins bearing non-catalytic cysteines (24-28). For example, afatinib, Ibrutinib, AMG-510 and PL pro inhibitor (SARS-Cov-2 PL^{pro}) are acrylamide based inhibitors of EGFR, Bruton's tyrosine kinase (BTK), K-Ras^{G12C} and respectively. Such irreversible inhibitors have the advantages of non-equilibrium kinetics, full target occupancy, and flexibility to modify the structure for absorption, distribution, metabolism, and excretion (ADME) issues without sacrificing potency and selectivity (29-31). The efficiency of a covalent inhibitor depends upon initial reversible binding with the protein and subsequent covalent bond formation with the target nucleophile. The former depends on its reversible binding kinetics whereas the latter depends on the reactivity of the electrophile and its accurate positioning. The intrinsic reactivity of acrylamides is significantly dictated by the nature of their amine precursor, which is complicated to modify without affecting the reversible binding of the ligand.

Furthermore, substitution at a or R positions usually reduces the reactivity of the acrylamides. On the other hand, electron-withdrawing groups (EWG) at the α-position increase the reactivity of the acrylamide while endowing reversibility to the formation of the covalent bond. The tunability of acrylamide reactivity is important for designing targeted covalent inhibitors. Recently, acrylamide analogs such as allenomides (29), alkynes (30), alkynyl benzoxazines, and dihydroquinazolines (3l) have been reported as covalent reactive groups. However, they differ significantly from acrylamides in their structure and geometry, and therefore the reactive moiety cannot be simply switched without requiring the modification of the reversible binding scaffold. Furthermore, the methacrylamides of this invention improved the efficiency (compared to known acrylamide analogs) towards the targeted protein and further, the methacrylamides of this invention have a releasing compound which can be used as a targeted drug delivery or as a turn on fluorescent/chemiluminescent probes.

This invention is directed to α-substituted methacrylamides as electrophilic warheads with varied reactivity, in the context of targeted covalent inhibitors. These compounds form a covalent bond with a nucleophile of a targeted of site-specific labelling of endogenous proteins, which may be followed by the concomitant release of a leaving group (FIGS. 1-3), such as a toxin, a fluorescent probe, a chemiluminescent probe a radiolabeled probe, a drug or any bio-active group. This invention is directed to Covalent Ligand Directed Release (CoLDR) Compounds providing a versatile addition to the toolbox of targeted covalent inhibitor design and able to modify various potential drug targets like BTK, K-Ras^{G12C}, and SARS-CoV-2 PL^{pro} different probes.

### SUMMARY

In one embodiment, this invention provides a Covalent Ligand Directed Releasing (CoLDR) Compound represented by the structure of formula I:

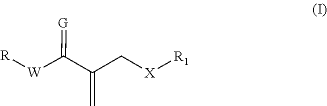

wherein:
R is a protein binding ligand, a fluorescent, a chemiluminescent probe, a radiolabeled probe or a bio-active group;
R₁ is a releasing group comprising a protein binding ligand, a fluorescent, a chemiluminescent probe, a radiolabeled probe or a bio-active group;
wherein R and R₁ are different and at least one of R and R₁ is a protein binding ligand;
W is a bond, NH, O, CH₂ or a linker;
G is O or S; and
X is a bond or a linker;
wherein, if X is a bond then R₁ is linked to the backbone structure directly via an ester bond, an amide bond, an anhydride bond, a carbamate bond, an oxygen atom, a sulfur atom or a nitrogen atom.

In one embodiment, this invention provides a prodrug comprising a Covalent Ligand Directed Releasing (CoLDR) Compound of this invention, wherein R is a protein bindingligand and R₁ is a drug or a targeted inhibitor, wherein, upon interaction between a protein and the protein bindingligand, the drug or the targeted inhibitor is released.

In one embodiment, this invention provides a protein sensor or a protein label comprising a Covalent Ligand Directed Releasing (CoLDR) Compound of this invention, wherein R or R₁ is a fluorescent probe or a chemiluminescent probe, wherein,
if R is a fluorescent probe or a chemiluminescent probe, and R₁ is a protein bindingligand; upon interaction between a protein and the protein binding ligand, the ligand is released and the fluorescent or the chemiluminescent probe is covalently attached to the protein and thereby results in change in fluorescence or chemiluminescence of the probe; or
if R is a protein binding ligand and R₁ is a fluorescent probe or a chemiluminescent probe, upon interaction between a protein and the protein binding ligand, the fluorescent probe or the or the chemiluminescent probe is released and the protein binding ligand is covalently attached to the protein and thereby results in change in fluorescence or chemiluminescence of the probes.

In one embodiment, this invention provides a protein proximity inducer compound comprising a Covalent Ligand Directed Releasing (CoLDR) Compound of this invention, wherein R is a protein binding ligand for the first protein and R₁ is another protein binding ligand for the second protein, wherein, upon interaction between the second protein and the its protein binding ligand, R₁ is released, and the second protein is then active and is labeled with R, inducing a new interaction with the first protein.

### BRIEF DESCRIPTION OF THE DRAWINGS

The subject matter regarded as the analog compounds and uses thereof is particularly pointed out and distinctly claimed in the concluding portion of the specification. The synthetic analog compounds and uses thereof, however, both as to organization and method of operation, together with objects, features, and advantages thereof, may best be understood by reference to the following detailed description when read with the accompanying drawings in which:

FIG. 1: Schematic illustration of the reaction of a target cysteine with a substituted α-methacrylamide through CoLDR (covalent ligand directed release) compounds. A refers to a protein binding ligand and B is a fluorescent/chemiluminescent/radiolabeled probe or a bio-active group, wherein B is released upon interaction with the protein.

FIG. 2: Schematic illustration of the reaction of a target cysteine with a substituted α-methacrylamide through CoLDR (covalent ligand directed release) compounds. A refers to a protein binding ligand and B is a fluorescent/chemiluminescent/radiolabeled probe or a bio-active group, wherein A is released upon interaction with the protein.

FIG. 3: Mechanism of turn-on chemiluminescence of compound 3k by BTK using CoLDR chemistry and subsequent dissociation pathway for the emission of a photon. Ibr refers to the following structure (Ibrutinib derivative):

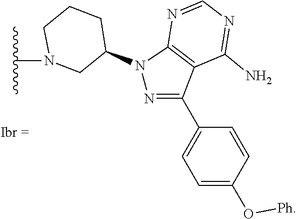

FIGS. 4A-4D: GSH reactivity correlates to the pKₐ of the leaving group. FIG. 4A: An example LC chromatogram shows monitoring of the reaction of 1g with GSH at 30 min (blue) and 48 h (green) GSH adduct: Retention time (RT)=4.3 min, m/z=480; coumarin: RT=4.5 min; reference: RT=4.8 min; 1g; RT=5.3 min; m/z=332. FIG. 4B: GSH t_{1/2} vs. pKₐ of the protonated leaving group (pK_{b} for amines; for 1j, pKₐ of carbonic acid is used). FIG. 4C: Fluorescence intensity of 1g as a function of incubation time with different GSH concentrations FIG. 4D: pH effect on the release and fluorescence of coumarin by 1g at a fixed GSH concentration (5 mM).

FIG. 5: Coumarin fluorescence as a function of GSH concentration. Coumarin's intrinsic fluorescence (Ex/Em=385/435 nm). Decreases with increasing GSH concentration as the pH is decreasing. Fluorescence of released coumarin from 1g initially increases as more thiol liberates the coumarin, but then decreases as the intrinsic fluorescence is decreased.

FIG. 6: Reaction of 100 μM 1g with GSH (0.5, 1 and 5 mM) as a function of time. Normalized % of GSH adduct is quantified by LC/MS. This shows that the release of coumarin is not decreasing with increasing GSH concentrations but only the fluorescence (FIG. 5).

FIG. 7: Effect of pH on the reaction of 5 mM GSH with 100 μM 1g after 24 hours.

FIGS. 8A-8B: α-methacrylamides show varied proteomic reactivity. FIG. 8A: Chemical structures of model electrophilic alkyne probes. FIG. 8B: In-situ proteomic labeling with the alkyne probes. Mino cells were treated for 2 h with either DMSO, IA-alkyne, or 2a-c, then lysed, “clicked” with TAMRA-azide, and imaged via in-gel fluorescence.

FIG. 9: Release of coumarin triggered by the addition of 5 mM GSH to 100 μM of either 1g and 2a at pH 8, shows almost identical release rates.

FIGS. 10A-10E: α-substituted derivatives of Ibrutinib as potential inhibitors: FIG. 10A: Chemical structures of the Ibrutinib derivatives. FIG. 10B: Time course LC-MS binding assay (2 μM compound and 2 μM BTK at room temperature). FIG. 10C: In vitro kinase activity assay using wild-type BTK (0.6 nM BTK, 5 μM ATP). FIG. 10D. GSH half-life (t_{1/2}) of Ibrutinib derivatives does not correlate to measured IC₅₀s FIG. 10E. Dose dependent inhibition of B cell response after anti-IgM-induced activation and treatment with Ibrutinib analogs for 24 h.

FIGS. 11A-11I: Turn-on fluorescent probes using CoLDR chemistry. FIGS. 11A-11C. Structures of turn-on fluorescent probes for BTK, EGFR, and K-Ras^{G12C} respectively. FIGS. 11D-11F. Time dependence of fluorescence intensity (representing the release of coumarin moiety) measured at Ex/Em=385/435 nm. Green curves show that the compounds in and of themselves (2 μM) are not fluorescent. Orange curves show that the proteins themselves (2 μM) are also not fluorescent. Only upon mixing of probe and target (blue curves) it shows an increase in fluorescence. FIGS. 11G-11I. Deconvoluted LC/MS spectra for BTK, EGFR, and K-Ras^{G12C} incubated with 3j, 4b, and 5a at the end of each plate reader measurement. The adduct mass corresponds to a labeling event in which the coumarin moiety was released, validating the proposed mechanism. For BTK (D) a reversible version of Ibrutinib Ibr-H was completed (2 μM; 0.5 h pre-incubation; FIG. 10A) with 3j (red curve). This considerably slowed the release of coumarin and the corresponding increase in fluorescence.

FIG. 12: Incubation of 3j with BTK at low equivalents (1 μM BTK; 50 nM 3j; Ex/Em=385/435 nm) shoed a detectable increase in fluorescence, but considerably slowed down the reaction, to a point that the initial kinetics can be observed.

FIGS. 13A-13B: Time dependence of turn-on fluorescence with 3j (Ex/Em=385/435 nm). FIG. 13A: 10 μM BSA with 2 μM 3j shows no reaction indicating the probes selectivity. FIG. 13B: 2 μM BTK fully labeled with IAA (red) compared to 2 μM non labeled BTK (blue) with 2 μM 3j. The lack of signal for the labeled BTK indicated the fluorescence is triggered by a free cysteine.

FIG. 14: EGFR kinase activity assay for two afatinib analogs 4a and 4b.

FIG. 15A-15D: Chemiluminescent BTK probe allows high throughput screening for BTK inhibitors. FIG. 15A. Structure of the chemiluminescent probe 3k; FIG. 15B: Time dependence of the luminescence signal (representing the release of chemiluminescent moiety). The compound in and of themselves (2 μM; green) is not luminescent. The protein itself (2 μm; orange) is also not luminescent. Only upon mixing of probe and target (blue) it shows an increase in luminescence. Pre incubation of BTK with a reversible version of Ibrutinib Ibr-H (2 μM; 0.5 h; red) inhibits luminescence. FIG. 15C: Schematic summary of % BTK binding inhibition in HTS using 3k shows an enrichment of known kinase inhibitors in the library to bind BTK compared to non-kinase inhibitors. FIG. 15D: Overall view of % BTK binding inhibition in the HTS. Known kinase inhibitors in red and known BTK inhibitors in Green.

FIG. 16A-16C: FIG. 16A: Structures of Ibrutinib and afatinib derivatives linked to toxins and chemotherapeutic compounds. FIG. 16B: LC-MS chromatogram shows the CoLDR chemistry releasing cargo's after reaction with BTK. C. Kinase activity of afatinib derivatives.

FIGS. 17A-17C: FIG. 17A: Ligand directed sites elective labeling of enzymes mechanism. FIG. 17B: structure of the Ibrutinib attached small molecule probes Figure C: Labelling of BTK with the alkyne, fluorescent, and copper-free alkyne compounds without ligand using LC-MS D. B-cell activation of Ibrutinib, 7d and 7f.

FIGS. 18A-18C: FIG. 18A. Structures of PHICs molecules and alkyne tagged NEDD4 inhibitors. FIG. 18B. LC-MS shows the labeling of BTK with PHICs molecules eliminating Ibr.

FIG. 19 presents fluorescence turn on results of compound 7m in the presence of, BTK (2 uM), KRAS (2 uM), BTK+Ibrutinib, BTK+Ibr-H thereby providing a turn-on fluorescence and can be used to label BTK in cells and keep it in active form.

FIG. 20A presents BTK activity in cells is not inhibited by (7d) and (7f). Mino cells were treated with 0.1% DMSO, 1 μM Ibrutinib-NH, 1 μM Ibrutinib-covalent, 100 nM (7d) or 100 nM (7f) for 1 hour. Half of the samples were washed ×3 times with cold-PBS. BTK activity was induced with g/ml anti-human IgM for 5 min, the cells were harvested, lysed and 50 ag of the lysates were then loaded on a 4-20% Bis-Tris gel. Immunoblots of phospho-BTK, total-BTK are presented.

FIG. 20B presents BTK half-life calculation using 7f. Mino cells were incubated with 100 nM 7f for 1 hour to pulse label BTK, washed ×3 times with cold-PBS and re-suspended with fresh medium. A sample of the cells was harvested at the indicated time-points. The cells were lysed, clicked to TAMRA-azide and imaged using Typhon FLA 9500 scanner at 532 nm. BTK levels were quantified with imageJ and half-life was calculated.

FIG. 21 presents synthetic schemes for the BTK labeling probes.

FIGS. 22A-22F present site-selective labeling of BTK using CoLDR chemistry. FIG. 22A—The chemical structure of the Ibrutinib attached methacrylamides with various functional probes. FIG. 22B—A typical example of reaction of BTK (2 μM) with 7n (2 μM) in 20 mM Tris buffer at pH 8, 25° C. FIG. 22C—Deconvoluted LC/MS spectra, shows the labeling of BODIPY probe and demonstrates Ibr-H leaving. FIG. 22D—% of labeling of BTK (2 μM) with the probes (7c, 7d, 7k, 7f, 7e, 7m, 7n, 7o, 7q, 7r, 7s; 2 μM) at 10, 30 and 120 min in 20 mM Tris buffer at pH 8, 25° C. FIG. 22E—Kinetics of the increase in fluorescence intensity measured at Ex/Em=550/620 nm (n=4) upon addition of BTK (2 μM) to 7m (2 μM) in 20 mM Tris buffer at pH 8, 37° C. (blue). Control experiments without BTK (red), preincubation of Ibrutinib (4 μM) and Ibr-H (4 μM) prior tp adding 7m (green and orange respectively) and incubation of K-Ras^{G12C} (pink) with 7m show no fluorescence. FIG. 22F—Deconvoluted LC/MS spectra for BTK incubated with 7m at the end of the fluorescence measurement (shown in 22E). The adduct mass corresponds to a labeling event in which the Ibr-H moiety was released, validating the proposed mechanism.

FIGS. 23A-23E present a reaction with reduced GSH validates the elimination of ligands and demonstrates their intrinsic thiol reactivity is within 2-fold of the parent acrylamide. FIG. 23A—A typical example of the reaction of GSH with 7n in 100 mM PBS buffer at pH 8, 10° C. FIG. 23B—An example LC chromatogram shows monitoring of the reaction of 7n (100 μM) with GSH (5 mM) at 0 h (blue) and 8 h (green) GSH adduct: Retention time (RT)=5.17 min, m/z=707; Ibr-H: RT=5.0 min; reference: RT=5.60 min; 7n; RT=5.38 min; m/z=786. UV absorption measured between 220-400 nm. FIG. 23C—Rates of depletion of Ibr-H derivatives (7d, 7f, 7e, 7m, 7n, 7q, 7r and 7s) in a reaction between 100 μM compound and 5 mM GSH in PBS buffer at pH 8, 37° C. (n=2) for 8 h. D. FIG. 23D—Rates of formation in LC-MS (absorption 220-400 nm) of Ibr-H, GSH adduct and depletion of 7n in a reaction between 100 μM 7n and 5 mM GSH in PBS buffer, pH 8, 37° C. (n=2). FIG. 23E—GSH t½ of all the probes and Ibrutinib.

FIGS. 24A-24G present selective labeling of various target proteins. FIGS. 24A, 24B, 24C—Structures of alkyne/ester labeling for BTK, K-Ras^{G12C} and SARS-CoV-2 PL^{Pro} respectively. FIG. 24D—Deconvoluted LC/MS spectra for BTK (2 uM) incubated with 7g (2 μM) in 20 mM Tris at pH 8, 25° C., 10 min. FIG. 24E—Deconvoluted LC/MS spectra for K-Ras^{G12C} (10 μM) incubated with 7h (100 μM) in 20 mM Tris at pH 8, 37° C., 16 h. FIG. 24F—Deconvoluted LC/MS spectra for PL^{Pro} (2 μM) incubated with 7t (10 μM) in 50 mM Tris at pH 8, 25° C., 16 h. The adduct mass corresponds to a labeling event in which the ligand was released. FIG. 24G—Synthesis route for e Evobrutinib alkyne 7g, 7h and 7t.

FIGS. 25A-25E present that labelling BTK with CoLDR probes does not inhibit its activity in cells. FIG. 25A. Cellular Labeling profile of 7d, 7f, and 7n after 2 h incubation with Mino cells and 7e in Mino cell lysate. 7d and 7f samples were further reacted with TAMRA-azide in lysate before imaging. An arrow indicates BTK's MW. FIG. 25B. Time-dependent labelling profile of 7f with BTK after incubation of Mino cells with 100 nM probe followed by click reaction with TAMRA-azide in lysate prior to imaging. FIG. 25C. Competition experiment of 7d, 7v, 7f and 7n with Ibrutinib. The cells were pre-incubated for 30 min with either 0.1% DMSO or 1 μM Ibrutinib, followed by 2 h incubation with 200 nM 7d, 7f or 100 nM 7v, 7n. FIG. 25D. Mino cells were incubated with 0.1% DMSO, 7d (100 nM) or preincubated with Ibrutinib (1 μM) then 7d (100 nM). Samples were further reacted with biotin-azide in lysate, followed by enrichment, trypsin digestion and peptide identification by LC/MS/MS. The Log(fold-ratio) of proteins enriched by 7d over DMSO is plotted as a function of statistical significance. BTK is clearly identified as the most enriched target, additional prominent targets that correspond to bands identified by in-gel fluorescence (FIG. 25C) are indicated. FIG. 25E. BTK activity assay in Mino cells as measured by autophosphorylation of BTK. The cells were incubated for 1 h with either 0.1% DMSO, 1 μM Ibrutinib, 1 μM Ibr-H or 100 nM 7d, 7f, 7m or 7n. The cells were either washed or not before induction of BTK activity by anti-IgM. FIG. 25F. BTK activity assay: Mino cells were incubated for 2 h with either DMSO, 1 μM 7d, 7f, 7n and 7m, washed, and then incubated for 45 min with Ibrutinib (100 nM). The cells were washed again before induction of BTK activity by anti-IgM. The CoLDR probes were able to rescue BTK activity from inhibition by Ibrutinib. FIG. 25G. Primary B cell activation induced by anti-IgM after 24 h treatment with increasing doses of either Ibrutinib, 7d or 7f showed no inhibition of the CoLDR probes.

FIG. 26A-26F present Measurement of BTK half-life. FIG. 26A. Half-life measurement of BTK using 7f. Mino cells were pulse-labelled with 100 nM 7f for 1 h and were then washed to remove the excess probe. Cells were harvested at the indicated time-points, and lysates were reacted with TAMRA-azide. The signal of BTK was quantified, and the half-life was calculated. FIG. 26B. Half-life measurement of BTK with cycloheximide (CHX) assay, using 20 μg/ml cycloheximide. FIG. 26C. Quantification of BTK levels in A and B (by normalization to the protein concentration) in Mino cells (7f: n=3, CHX: n=4). FIG. 26D. Calculated half-life by both methods, presented as mean±SD. FIG. 26E. Degradation of BTK labelled with 7m using PROTAC 9d. Mino cells were incubated with 7m (100 nM), and then washed to remove the excess probe and again incubated with PROTAC 9d for 2 h at 0.5 μM and 1 μM and then lysed. Samples were subjected to in-gel fluorescence (FL) and western blot (WB). FIG. 26F. Quantification of BTK levels in panel 26E (normalization to the β-actin has been done for western blot).

FIG. 27 presents synthetic scheme for the preparation of PROTACs.

FIG. 28 presents turn-on fluorescent environmental sensitive probe detecting binding events to BTK. FIG. 28A—Fluorescence spectrum scan of 7m (2 μM) in the presence/absence of BTK (2 μM). Inset shows the normalized fluorescence spectrum, where it is evident there is a shift in the peak upon protein binding. FIG. 28B—Dose dependent reduction of the fluorescence, and shift of the peak emission of BTK labelled 7m after the addition of excess ligands (Ibrutinib and Ibr-H). FIG. 28C—Three-fold increase in the fluorescence intensity of 7n (2 μM) when incubated with BTK (2 μM) and reduction of the fluorescence after the addition of excess ligand. FIG. 28D—Changes in the fluorescence intensity of 7e (2 μM) after the addition of BTK (2 μM) followed by Ibrutinib and Ibr-H. FIG. 28E—Fluorescence scan of BTK labelled 7m (2 μM) incubated with various BTK binders shows more than 2.5 fold change in the 650/620 emission ratio. FIG. 28F—BTK inhibitors caused significant quenching of fluorescence of BTK-7m.

FIG. 29 Presents labelling by CoLDR probes does not affect ligand binding. FIG. 29A. Structure of the Ibrutinib based reversible compound used to label the SPR chip. FIGS. 29B-29D. Surface plasmon resonance (SPR) sensorgrams for (29B) BTK, (29C) BTK-7d and (29D) BTK-ibrutinib at different concentrations. FIG. 29E. Kinetic parameters for association (ka) and dissociation constants (kd) for BTK and BTK-1b. SE=Standard Error.

FIG. 30: presents measurement of induced degradation by CoLDR PROTACs. FIG. 30A. Schematic representation of target degradation using CoLDR PROTACS. FIG. 30B. Structure of CoLDR based BTK PROTACS. FIG. 30C. In vitro labelling of BTK (2 μM) with 9a-9c (2 μM) in 20 mM Tris buffer at pH 8, 37° C. FIG. 30D. Western blot evaluation of BTK levels in Mino cells in response to various concentrations of 9c after 24 h of incubation. FIG. 30E. Quantification of BTK levels in (FIG. 30D) by normalization to the j-actin house-keeping gene in Mino cells. DC₅₀ and Dₘₐₓ were calculated by fitting the data to a second-order polynomial using the Prism software. FIG. 30F. Mino cells were pre-treated for 2 h with either Ibrutinib/thalidomide-OH or DMSO before treatment with a BTK PROTAC for 24 h (n=2). Subsequently, BTK levels were measured via Western blot. FIG. 30G. Mino cells were treated for 24 h with either 0.1% DMSO or 9c (500 nM) in 4 replicates. Lysates were subjected to trypsin digestion and peptide identification by LC/MS/MS. The Log₂ (fold-ratio) of proteins enriched in the DMSO samples over 9c treated samples is plotted as a function of statistical significance. Significantly degraded proteins are indicated in red and defined as Log₂ (DMSO/9c) >1 and p-value <0.01.

FIGS. 31A-F: presents fluorescent labelling does not inhibit active site binding and ternary complex formation. FIG. 31A. Schematic representation of protein labelled with CoLDR probe followed degradation with PROTAC. FIG. 31B. Structure of reversible PROTAC 9d.

FIGS. 31C, 31D, 31E. Mino cells were treated with 7n for 1 h washed and incubated with 9d at various concentrations. Degradation was measured using in-gel fluorescence (FIGS. 31C and 31E) and Western blot (FIG. 31D). FIG. 31F. BTK degradation by 9d at 50, 100, 500 nM measured using Western Blot.

It will be appreciated that for simplicity and clarity of illustration, elements shown in the figures have not necessarily been drawn to scale. Further, where considered appropriate, reference numerals may be repeated among the figures to indicate corresponding or analogous elements.

### DETAILED DESCRIPTION

This invention is directed to α-substituted methacrylamides compounds as electrophilic warheads with varied reactivity, in the context of targeted covalent inhibitors.

The α-substituted methacrylamides compounds of this invention are Covalent Ligand Directed Releasing (CoLDR) Compounds possessing (1) a protein binding ligand and (2) a fluorescent, a chemiluminescent, a radiolabeled probe, or any bio-active group; wherein, based on the design of the Covalent Ligand Directed Releasing (CoLDR) Compound,
the protein binding ligand is covalently linked to a protein and the fluorescent, the chemiluminescent or the radiolabeled probe, or any bio-active group is released, upon binding to the protein; or
the fluorescent, the chemiluminescent or the radiolabeled probe, or any bio-active group is covalently linked to the protein and the protein binding ligand is released, upon binding to the protein.

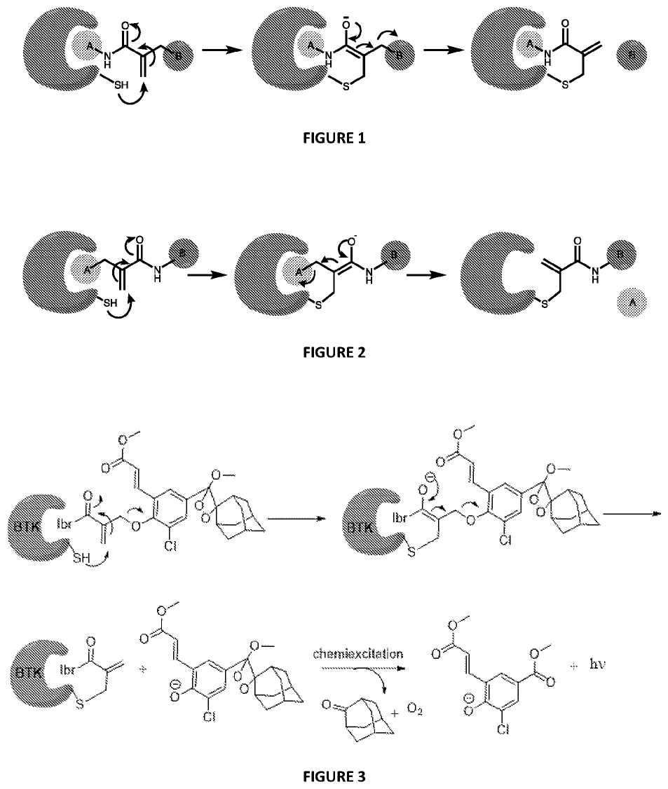

These compounds form a covalent bond with a nucleophile of a targeted protein via addition-elimination reaction upon, which may be followed by the concomitant release of a leaving group (i.e. R₁ of compound of formula I). (FIGS. 1-3).

The Covalent Ligand Directed Releasing (CoLDR) Compounds of this invention can be used to modulate the reactivity of selective covalent inhibitors, sensors, diagnostics or can be used as turn-on probes against proteins.

In one embodiment, this invention is directed to a Covalent Ligand Directed Releasing (CoLDR) Compound represented by the structure of formula I:

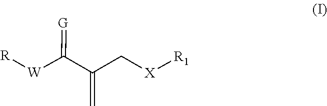

wherein:
R is a protein binding ligand, a fluorescent, a chemiluminescent, a radiolabeled probe or a bio-active group;
R₁ is a releasing group comprising a protein binding ligand, a fluorescent, a chemiluminescent, a radiolabeled probe or a bio-active group;
wherein R and R₁ are different and at least one of R and R₁ is a protein binding ligand;
W is a bond, NH, O, CH₂, or a linker;
G is O or S; and
X is a bond or a linker;
wherein, if X is a bond then R₁ is linked to the backbone structure directly via an ester bond, an amide bond, an anhydride bond, a carbamate bond, an oxygen atom, a sulfur atom or a nitrogen atom.

In one embodiment, this invention is directed to a Covalent Ligand Directed Releasing (CoLDR) Compound represented by the structure of formula IA:

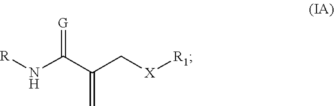

wherein:
R is a protein binding ligand, a fluorescent, a chemiluminescent, a radiolabeled probe or a bio-active group;
R₁ is a releasing group comprising a protein binding ligand, a fluorescent, a chemiluminescent, a radiolabeled probe or a bio-active group;
wherein R and R₁ are different and at least one of R and R₁ is a protein binding ligand;
G is O or S; and
X is a bond or a linker;
wherein, if X is a bond then R₁ is linked to the backbone structure directly via an ester bond, an amide bond, an anhydride bond, a carbamate bond, an oxygen atom, a sulfur atom or a nitrogen atom.

In one embodiment, this invention is directed to a Covalent Ligand Directed Releasing (CoLDR) Compound represented by the structure of formula IB:

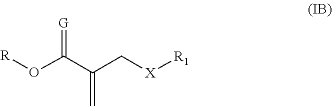

wherein:
R is a protein binding ligand, a fluorescent, a chemiluminescent, a radiolabeled probe or a bio-active group;
R₁ is a releasing group comprising a protein binding ligand, a fluorescent, a chemiluminescent, a radiolabeled probe or a bio-active group;
wherein R and R₁ are different and at least one of R and R₁ is a protein binding ligand;
G is O or S; and
X is a bond or a linker;
wherein, if X is a bond then R₁ is linked to the backbone structure directly via an ester bond, an amide bond, an anhydride bond, a carbamate bond, an oxygen atom, a sulfur atom or a nitrogen atom.

In one embodiment, this invention is directed to a Covalent Ligand Directed Releasing (CoLDR) Compound represented by the structure of formula IC:

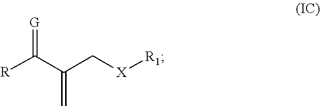

wherein:
R is a protein binding ligand, a fluorescent, a chemiluminescent, a radiolabeled probe or a bio-active group;
R₁ is a releasing group comprising a protein binding ligand, a fluorescent, a chemiluminescent, a radiolabeled probe or a bio-active group;
wherein R and R₁ are different and at least one of R and R₁ is a protein binding ligand;
G is O or S; and
X is a bond or a linker;
wherein, if X is a bond then R₁ is linked to the backbone structure directly via an ester bond, an amide bond, an anhydride bond, a carbamate bond, an oxygen atom, a sulfur atom or a nitrogen atom

In some embodiments, the Covalent Ligand Directed Releasing (CoLDR) Compound of this invention comprises: (1) a protein binding ligand and (2) a fluorescent, a chemiluminescent, a radiolabeled probe, a hydrophobic tag, a bio-active group or a second protein binding ligand.

In some embodiments, the Covalent Ligand Directed Releasing (CoLDR) Compound of this invention comprises a bio-active group. In other embodiments, the bio-active group includes, but not limited to an approved drug, a targeted inhibitor, a cytotoxic, a chemotherapeutic, amino acid side chains, a protein binding ligand, a radiopharmaceutical, substructure or derivative thereof or any chemical modification that elicits a biological perturbation.

“Targeted Inhibitor” as referred herein is a small molecule that shows selective binding of a specific protein or specific protein family. Non limiting examples of targeted inhibitor include: AMG-510, CCT251545, A-366, CPI-169, T0901317, BAY-3827, CM11, Veliparib, BI-1935, SD-36, XMD-12, TH5427, AMG232, 25CN-NBOH, GSK2334470, UNC0642, MRK-740, GSK343, BYL-719, MK-5108, R05353, AX15836, PD0332991, EPZ015666, Luminespib, CPI-360, OICR-9429, PT2399, S63845, Venetoclax, THZ531, CGI1746, (R)-PFI-2, MI-77301, EPZ004777, Linsitinib, Ruxolitinib, FS-694, CPI-0610, CP-724714, GSK481, BTZO-1, MT1, MS023, SCH772984, BAY-1816032, FM-381, Niraparib, UNC1215, SR-318, MRTX849, A-196, CCT251236, JQ1, CH5424802, AT1, BAY-598, UCSF7447, AM-6761, VX-745, PFI-1, PFI-3, GSK4027, SGC0946, SGC707, EED226, BGJ-398, BLU9931, Tofacitinib, GDC-0879, P505-15, PF-CBP1, AMG900, Skepinone-L, AZD2014, GSK484, CHIR-99021, (R)-9s, UCSF4226, NVS-PAK1-1, EI1, KZR-504, AZD1152, SGX-523, CCT241533, RG7388, VH298, PF-477736, BMS-911543, AB680, BAY1125976, GSK583, BI-2545, EPZ-5676, G-5555, A-395, GNF-5, Romidepsin, EPZ011989, ULK-101, THPP-1, D0264, BAY-707, MZ1, UNC1999, WEHI-539, NVP-AEW541, THZ1, AMG-18, JNK-IN-8, BiBET, EPZ-6438, GSK-J4, CCT244747, CPI-1612, KI-696, PF3644022, SGC-CBP30, Tubacin, Selumetinib, Rapamycin, GSK591, ML323, ABBV-744, AC220, Talazoparib, PDD00017273, Filgotinib, A-485, RG7112, BAZ2-ICR, MI-888, BMX-IN-1, BI-9564, PF-3758309, BAY-985, MCC950, UNC2025, AZD-6482, RGFP966, Bistramide A, Ogerin, I-BRD9, I-CBP112, Eleutherobin, GSK864, Salvinorin A, MLi-2, ICI-199441, BIX-02188, Olaparib, A-1155463, WZ4003, KH-CB19, Tubastatin A, AMG 176, eCF309, E7449, AZ191, BAY-826, R02468, ABT-100, XMD8-87, NI-57, NMS-P118, GW3965, eCF506, ACY-738, BAY-549, HG-9-91-01, WM-1119, T-26c, AZ6102, Glyburide, Pevonedistat, GNE7915, Relacatib, Bafetinib, Pictilisib, Afatinib, VE-821, A-1210477, AVL-292, XMD8-92, RUSKI-201, UNC3866, MPS1-IN-1, GNE-2861, ST0609, AZ0108, I-BET151, BAY-885, 2-MT 63, DDR1-IN-1, EPZ020411, CPI-1205, TP-004, Repaglinide, L-Moses, LXR-623, GSK-5959, CPI-637, GPR40ant39, UNC0638, GSK2801, M-808, JAK3i, CX-4945, RSL3, BAY-299, Cotransin, MIV-6R, CP-673451, AC-4-130, LLY-507, ABPA3, TP-020, PF-4800567, Englerin A, LP99, JQEZ5, BI2536, AGI-6780, KU-60019, DS-437, BMS-265246, CMLD-2, BI-D1870, AGI-5198, WH-4-023, Cortistatin A, NI-42, BIX-01294, TX1-85-1, CFI-400945, (R)-Zinc-3573, URMC-099, XAV939, JW55, TTT20171, Imatinib, dTRIM24, MBM-55, MZP-54, TBK1 PROTAC 3i, GNE-049, WZ4002, NCT-505, SR9238, U18666A, NIK SMIl, TL13-112, GSK2982772, MD-224, LNP-023, AMG-337, MK-8033, AZD3988, RU.521, dBET6, ARS-1620, MLT-748, GDC-0834, LSN 3213128, GSK2033, PT2385, Adavosertib, VZ185, GSK2194069, MG-277, TAK-243, A-770041, GNF-5837, GSK2973980A, THAL-SNS-032, dTAG-13, GNE-781, EML631, QC6352, Capmatinib, PF-06869206, BSJ-03-123, Asciminib, SB-612111 or TH1760, TP-024.

“An approved drug” as referred herein is any chemical entity the received the U.S. Food and Drug Administration, China Food and Drug Administration, European Medicines Agency, or any regulatory agency, approval for usage in human.

“A toxin” and “A cytotoxic” as referred herein is a compound with non-selective cell killing activity.

Non limiting examples of “A chemotherapeutic” include: Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vemurafenib, Vinblastine, Vincristine or Vindesine.

“A radiolabeled probe” or “radiopharmaceuticals” include any probe or pharmaceutical, respectively which possess a radioactive isotope. Non limiting examples of radiopharmaceuticals include: 177Lu-PSMA-617 (lutetium Lu 177 vipivotide tetraxetan). 177 Lu PSMA-617 is a radiolabeled drug that target prostate-specific membrane antigen (PSMA) in prostate cancer. PSMA is a membrane bound glycoprotein which is over expressed in prostate cancer. Lutetium-177 once internalized into the cell irreversibly sequestered within the targeted tumor cell. It emits radiation over a millimeter range that is ideal for eradication of the cancer cells. The therapeutic candidate acts by binding to the PSMA expressing cancer cells and exhibit cytotoxicity. Lutetium Lu-177 dotatate or Lutetium (177Lu) oxodotreotide (Lutathera): Lutetium Lu 177 dotatate binds to somatostatin receptors with highest affinity for subtype 2 receptors (SSRT2). Upon binding to somatostatin receptor expressing cells, including malignant somatostatin receptor-positive tumors, the compound is internalized. The beta emission from Lu 177 induces cellular damage by formation of free radicals in somatostatin receptor-positive cells and in neighboring cells. Radium-223 chloride (Xofigo): The active moiety of radium Ra 223 dichloride is the alpha particle-emitting isotope radium-223, which mimics calcium and forms complexes with the bone mineral hydroxyapatite at areas of increased bone turnover, such as bone metastases. The high linear energy transfer of alpha emitters (80 keV/micrometer) leads to a high frequency of double-strand DNA breaks in adjacent cells, resulting in an anti-tumor effect on bone metastases. The alpha particle range from radium-223 dichloride is less than 100 micrometers (less than 10 cell diameters) which limits damage to the surrounding normal tissue.

In some embodiments, the Covalent Ligand Directed Releasing (CoLDR) Compound of this invention comprises a fluorescent, a chemiluminescent or a radiolabeled probe. In other embodiments, the fluorescent probe comprises non limited examples of rhodamine, cyanine, coumarin, Nile red, Nile blue, dansyl, umberiferon, bodipy, environment sensitive fluorophore or derivative thereof. In other embodiments, the chemiluminescent probe comprises dioxetane-based compounds, 2,3-dihydrophthalazinedione such as luciferin and luminol or derivative thereof. In other embodiments the radiolabeled probe includes any ligand possessing a radioactive isotope.

In some embodiment, the Covalent Ligand Directed Releasing (CoLDR) Compound of this invention comprise a protein binding ligand. In another embodiment, the protein binding ligand comprises any acrylamide-based or vinylsulfone based or α,β unsaturated carbonyl based protein inhibitor or analog thereof. In another embodiment, the protein binding ligand comprises afatinib, Ibrutinib, Evobrutinib, AMG-510, PL pro inhibitor or derivatives thereof. In another embodiment, a non-limiting example of a protein binding ligand is afatinib or poziotinib or osimertinib or neratinib and its targeted protein is EGFR. In another embodiment, a non-limiting example of a protein binding ligand is Ibrutinib or zanubrutinib or evobrutinib or remibrutinib or spebrutinib and its targeted protein is BTK or BLK. In another embodiment, a non-limiting example of a protein binding ligand is AMG-510 or ARS-1620 or MRTX849 and its targeted protein is K-Ras^{G12C}. In another embodiment, a non-limiting example of a protein binding ligand is PF-06651600 and its protein target is JAK3. In another embodiment, a non-limiting example of a protein binding ligand is Futibatinib or FIIN1 or FIIN2 or FIIN3, PRN1371 and its protein target is FGFR. In another embodiment, a non-limiting example of a protein binding ligand is NU6300 and its protein target is CDK2. In another embodiment, a non-limiting example of a protein binding ligand is THZ1 and its protein target is CDK7. In another embodiment, a non-limiting example of a protein bindingligand is THZ531 and its protein target is CDK12 or CDK13. In another embodiment, a non-limiting example of a protein binding ligand is CNX-1351 and its protein target is PI3Kα. In another embodiment, a non-limiting example of a protein binding ligand is JNK-IN-8 (or derivatives or analogs thereof) and its protein target is JNK. In another embodiment, a non-limiting example of a protein binding ligand is MKK7-COV-3 (or derivatives or analogs thereof) and its protein target is MKK7. In another embodiment, a non-limiting example of a protein binding ligand is CC-90003 and its protein target is ERK1 or ERK2. In another embodiment, a non-limiting example of a protein binding ligand is E6201 and its protein target is MEK1.

In some embodiments, the Covalent Ligand Directed Releasing (CoLDR) Compound of this invention is presented by the structures of formula I, IA, IB or IC. In other embodiments, R₁ of the structures of formula I, IA, IB or IC, is a releasing group, wherein upon interaction between a protein and the protein target ligand of the Covalent Ligand Directed Releasing (CoLDR) Compound, R₁ is released. In another embodiment, if R₁ is a protein binding ligand, then, the protein binding ligand of R₁ is released.

In some embodiments, R of the structures of formula I, IA, IB or IC is a protein binding ligand, and R₁ is a fluorescent, a chemiluminescent or a radiolabeled probe. In another embodiment, R of the structures of formula I, IA, IB or IC is a protein binding ligand and R₁ is a fluorescent, a chemiluminescent or a radiolabeled probe, wherein R₁ (the fluorescent, chemiluminescent or the radiolabeled probe) is released upon binding to the protein, while the protein binding ligand is covalently linked to the protein. In another embodiment, the covalent bond is formed via a nucleophilic moiety of the protein and the double bond (—C═CH₂) of the compound structures of formula I, IA, IB or IC. In another embodiment, the nucleophilic moiety of the protein is a thiol, an amine, or a hydroxyl.

In some embodiments, R of the structures of formula I, IA, IB or IC is a protein binding ligand, and R₁ is a bio-active group. In another embodiment, R of the structures of formula I, IA, IB or IC is a protein binding ligand and R₁ is a bio-active group, wherein R₁ (the bio-active group) is released upon binding to the protein, while the protein binding ligand is covalently linked to the protein. In another embodiment, the covalent bond is formed via a nucleophilic moiety of the protein and the double bond (—C═CH₂) of the compound structures of formula I, IA, IB or IC. In another embodiment, the nucleophilic moiety of the protein is a thiol, an amine, or a hydroxyl.

In some embodiments, R of the structures of formula I, IA, IB or IC is a fluorescent, a chemiluminescent or a radiolabeled probe, and R₁ is a protein binding ligand. In another embodiment, R of the structures of formula I, IA, IB or IC is a fluorescent, a chemiluminescent or a radiolabeled probe and R₁ is a protein binding ligand, wherein R₁ (the protein binding ligand) is released upon binding to the protein, while the fluorescent, chemiluminescent or the radiolabeled probe is covalently linked to the protein. In another embodiment, the covalent bond is formed via a nucleophilic moiety of the protein and the double bond (—C═CH₂) of the compound structures of formula I, IA, IB or IC. In another embodiment, the nucleophilic moiety of the protein is a thiol, an amine, or a hydroxyl.

In some embodiments, R of the structures of formula I, IA, IB or IC is a bio-active group and R₁ is a protein binding ligand. In another embodiment, R of the structures of formula I, IA, IB or IC is a bio-active group and R₁ is a protein binding ligand, wherein R₁ (the protein binding ligand) is released upon binding to the protein, while the bio-active group is covalently linked to the protein. In another embodiment, the covalent bond is formed via a nucleophilic moiety of the protein and the double bond (—C═CH₂) of the compound structures of formula I, IA, IB or IC. In another embodiment, the nucleophilic moiety of the protein is a thiol, an amine, or a hydroxyl.

In some embodiments, R of the structures of formula I, IA, IB or IC is a protein binding ligand for a first protein and R₁ is a protein binding ligand for a second protein. In another embodiment, R of the structures of formula I, IA, IB or IC is a protein binding ligand for the first protein and R₁ is a protein binding ligand for the second protein, wherein R₁ (the protein binding ligand for the second protein) is released upon interaction to the second protein, while the protein binding ligand for the first protein is covalently linked to the first protein. In another embodiment, the covalent bond is formed via a nucleophilic moiety of the protein and the double bond (—C═CH₂) of the compound structures of formula I, IA, IB or IC. In another embodiment, the nucleophilic moiety of the protein is a thiol, an amine, or a hydroxyl.

In some embodiments, X as defined in the structures of Formula I, IA, IB or IC is a linker or a bond. In other embodiments, X is a bond. In other embodiments, X is a linker. In other embodiments, the linker comprises an alkyl, a cycloalkyl, a heterocycloalkyl, an aryl, a heteroaryl, an ester bond, an amide bond, a carbamate bond, an anhydride bond, an oxygen atom, an amine, a sulfur atom, a nitrogen atom, a dendrimer, a self immolative linker, a PEG or combination thereof. In another embodiment the linker is alkylene diamine. In another embodiment the linker is —N-alkyl-N, N-alkyl-C(O)N—, —N-alkyl-N(CO)—, —N-alkyl-O—C(O)—N—, —OC(O)-alkyl-N—, —OC(O)-alkyl-C(O)N—, —OC(O)-alkyl-N(CO)—, —OC(O)-alkyl-O—C(O)—N—, —C(O)O-alkyl-N—, —C(O)O-alkyl-C(O)N—, —C(O)O-alkyl-N(CO)—, —C(O)O-alkyl-O—C(O)—N—, —O—(CO)—N-alkyl-C(O)N, —O—(CO)—N-alkyl-NC(O)—, —O—(CO)—N-alkyl-N—, —O—C(O)—N-alkyl-O—C(O)—N—; wherein the nitrogen (N) and the alkyl can be optionally substituted. In another embodiment the linker is a self immolative linker. In another embodiment the linker is a dendrimer. In another embodiment the linker is a PEG.

In other embodiments, if wherein, if X of the structures of Formula I, IA, IB or IC is a bond then R₁ is linked to the backbone structure directly via an ester bond, an amide bond, an anhydride bond, a carbamate bond, an oxygen atom, a sulfur atom or a nitrogen atom. Each is a separate embodiment of this invention.

In some embodiments, G as defined in the structures of Formula I, IA, IB or IC is an oxygen atom (O) or a sulfur atom (S). In other embodiments, G is an oxygen atom (O). In other embodiments, G is a sulfur atom (S).

In some embodiments, W as defined in the structures of Formula I is a bond, NH, an oxygen atom (O), CH₂ or a linker. In other embodiments, W is a bond. In other embodiments, W is a NH. In other embodiments, W is an oxygen atom (O). In other embodiments, W is a CH₂. In other embodiments, W is a linker. In other embodiments, the linker comprises an alkyl, a cycloalkyl, a heterocycloalkyl, an aryl, a heteroaryl, an ester bond, an amide bond, a carbamate bond, an anhydride bond, an oxygen atom, an amine, a sulfur atom, a nitrogen atom, a dendrimer, a self immolative linker, a PEG or combination thereof. In another embodiment the linker is alkylene diamine. In another embodiment the linker is —N-alkyl-N, N-alkyl-C(O)N—, —N-alkyl-N(CO)—, —N-alkyl-O—C(O)—N—, —OC(O)-alkyl-N—, —OC(O)-alkyl-C(O)N—, —OC(O)-alkyl-N(CO)—, —OC(O)-alkyl-O—C(O)—N—, —C(O)O-alkyl-N—, —C(O)O-alkyl-C(O)N—, —C(O)O-alkyl-N(CO)—, —C(O)O-alkyl-O—C(O)—N—, —O—(CO)—N-alkyl-C(O)N, —O—(CO)—N-alkyl-NC(O)—, —O—(CO)—N-alkyl-N—, —O—C(O)—N-alkyl-O—C(O)—N—; wherein the nitrogen (N) and the alkyl can be optionally substituted. In another embodiment the linker is a self immolative linker. In another embodiment the linker is a dendrimer. In another embodiment the linker is a PEG.

In some embodiments, this invention is directed to a prodrug, wherein the prodrug comprises a Covalent Ligand Directed Releasing (CoLDR) Compound represented by the structures of Formula I, IA, IB or IC of this invention, wherein R is a protein binding ligand and R₁ is a drug or a targeted inhibitor, or a toxin, or a radiopharmaceutical, or a chemotherapeutic wherein, upon interaction between a protein and the protein binding ligand, the drug or the targeted inhibitor or the toxin or the chemotherapeutic is released.

In some embodiments, provided herein a pharmaceutical composition comprising a prodrug Covalent Ligand Directed Releasing (CoLDR) Compound represented by the structures of Formula I, IA, IB or IC, wherein R is a protein binding ligand and R₁ is a drug, a radiopharmaceutical, a targeted inhibitor, a toxin or a chemotherapeutic and a pharmaceutical acceptable carrier.

In another embodiment, a covalent bond is formed between the protein and the protein binding ligand of the Covalent Ligand Directed Releasing (CoLDR) Compounds provided herein. In another embodiment, a covalent bond is formed via a nucleophilic moiety of the protein and the double bond (—C═CH₂) of the CoLDR compounds provided herein. In another embodiment, the nucleophilic moiety of the protein is a thiol, an amine, or a hydroxyl.

In some embodiments, this invention provides a protein sensor or a protein label comprising a Covalent Ligand Directed Releasing (CoLDR) Compound represented by the structures of Formula I, IA, IB or IC of this invention, wherein R or R₁ is a fluorescent probe or a chemiluminescent probe, wherein,
if R is a fluorescent probe or a chemiluminescent probe, and R₁ is a protein binding ligand; upon interaction between a protein and the protein binding ligand, the protein binding ligand is released and the fluorescent or the chemiluminescent probe is covalently attached to the protein and thereby results in change in fluorescence or chemiluminescence of the probe (FIG. 2, where A is a fluorescent or the chemiluminescent probe a and B is protein binding ligand); or
if R is a protein binding ligand and R₁ is a fluorescent probe or a chemiluminescent probe, upon interaction between a protein and the protein binding ligand, the fluorescent probe or the or the chemiluminescent probe is released and the protein binding ligand is covalently attached to the protein and thereby results in change in fluorescence or chemiluminescence of the probes. (FIG. 1, where A is a protein binding ligand and B is fluorescent or the chemiluminescent probe).

In some embodiments, this invention provides a protein sensor or a protein label comprising a Covalent Ligand Directed Releasing (CoLDR) Compound represented by the structures of Formula I, IA, IB or IC of this invention, wherein R or R₁ is a radiopharmaceutical probe, wherein,
if R is a radiopharmaceutical probe, and R₁ is a protein binding ligand; upon interaction between a protein and the protein binding ligand, the protein binding ligand is released and the radiolabeled probe is covalently attached to the protein and thereby the protein can be diagnosed/sensed (FIG. 2, where A is radiolabeled probe a and B is protein binding ligand); or
if R is a protein binding ligand and R₁ is radiolabeled probe, upon interaction between a protein and the protein binding ligand, the radiolabeled probe is released and the protein binding ligand is covalently attached to the protein and thereby the protein can be diagnosed/sensed. (FIG. 1, where A is a protein binding ligand and B is radiolabeled probe).

In another embodiment, a covalent bond is formed between the protein and the protein binding ligand. In another embodiment, a covalent bond is formed via a nucleophilic moiety of the protein and the double bond (—C═CH₂) of the compounds of formula I, IA, IB or IC. In another embodiment, the nucleophilic moiety of the protein is a thiol, an amine, or a hydroxyl.

Exemplary specific compounds of the Compounds of I, IA-IC of this invention are represented in the following table:

| Protein binding | | Bio-active group/ |
|---|---|---|
| ligand | Structure | Labeling probe/drug |
| Ibrutinib (BTK inhibitor) | 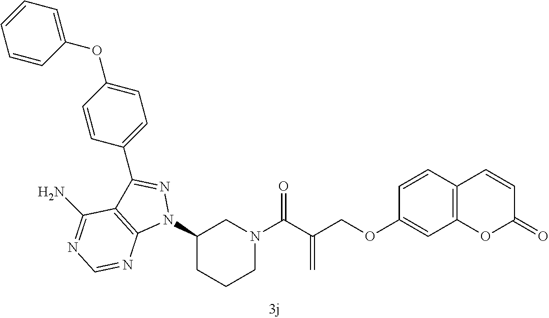 [SMILES: C=C(COc1ccc2ccc(=O)oc2c1)C(=O)N1CCC[C@@H](n2nc(-c3ccc(Oc4ccccc4)cc3)c3c(N)ncnc32)C1] | coumarin |
| Ibrutinib | 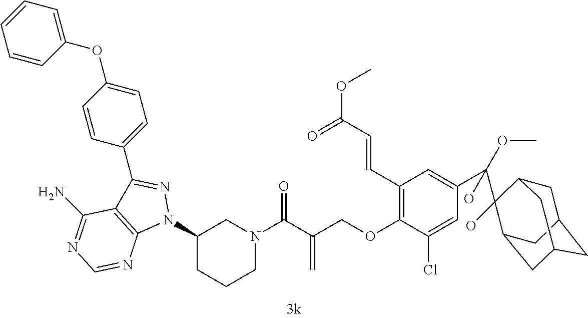 [SMILES: C=C(COc1c(Cl)cc(C(O)(OC)C2(O)C3CC4CC(C3)CC2C4)cc1/C=C/C(=O)OC)C(=O)N1CCC[C@@H](n2nc(-c3ccc(Oc4ccccc4)cc3)c3c(N)ncnc32)C1] | Chemiluminescent probe |
| Ibrutinib | 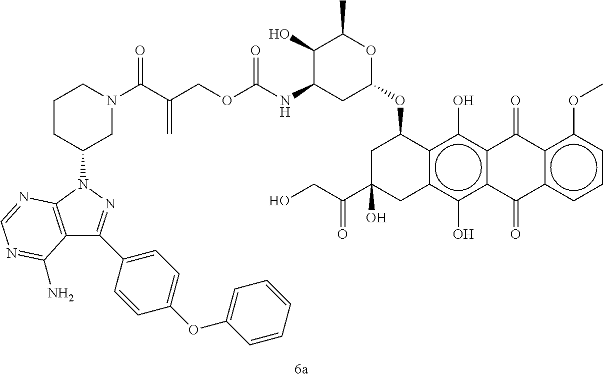 | Doxorubicin |
| Ibrutinib | 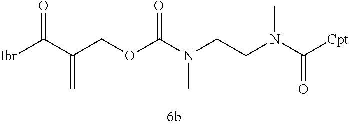 | Camptothecin |
| Ibrutinib | 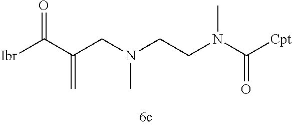 | Camptothecin |
| Ibrutinib | 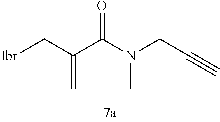 | alkyne |
| Ibrutinib | 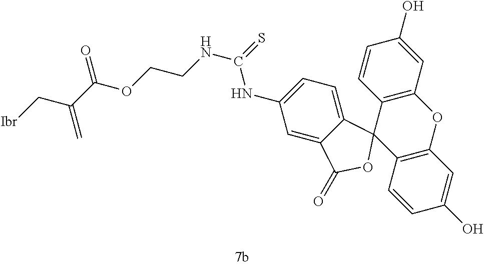 | FITC |
| Ibrutinib | 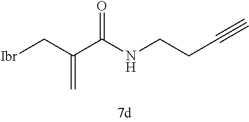 | alkyne |
| Ibrutinib | 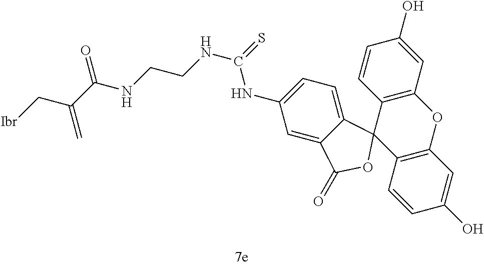 | FITC |
| Ibrutinib | 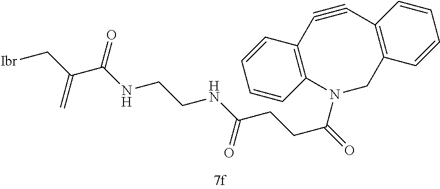 | dibenzyl cyclooctyne |
| Ibrutinib | 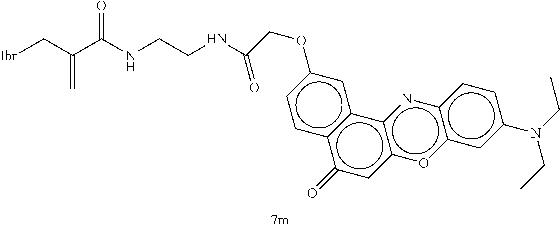 | Nile red |
| Ibrutinib | 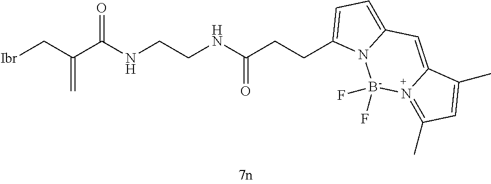 | BODIPY |
| Ibrutinib | 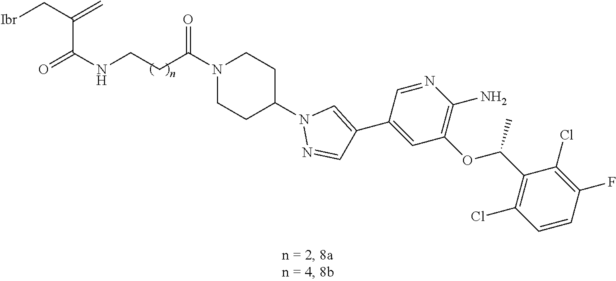 | crizotinib |
| Ibrutinib | 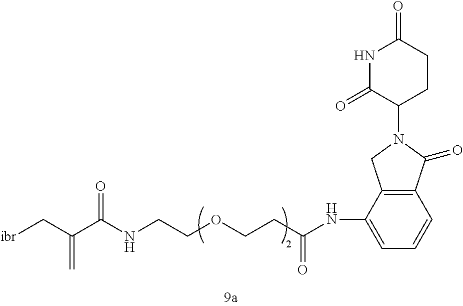 | Pomalidomide |
| Ibrutinib | 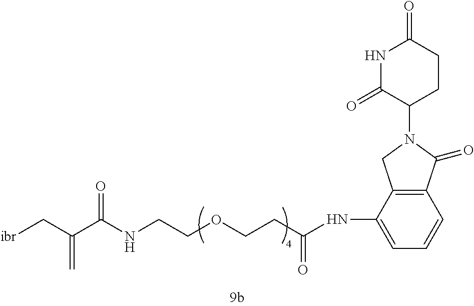 | Pomalidomide |
| Ibrutinib | 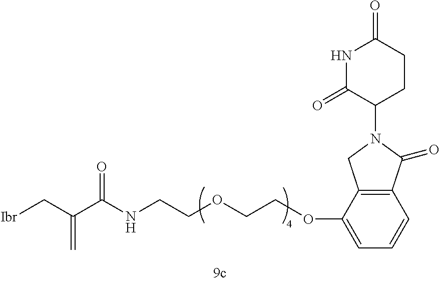 | thalidomide |
| Ibrutinib | 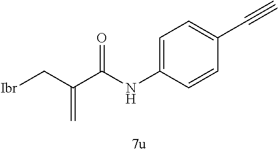 | alkyne |
| Ibrutinib | 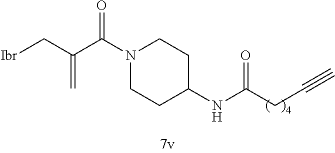 | alkyne |
| Ibrutinib | 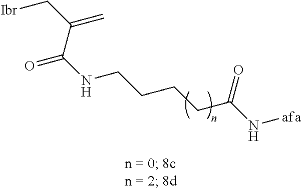 | afatinib |
| Ibrutinib | 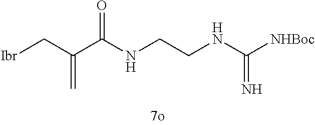 | Boc guanidine |
| Ibrutinib | 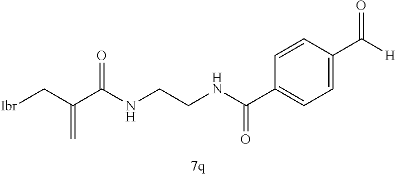 | aromatic aldehyde |
| Ibrutinib | 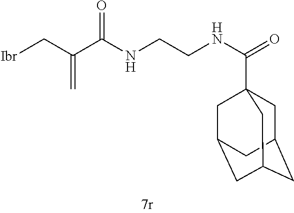 | hydrophobic adamantine |
| Ibrutinib | 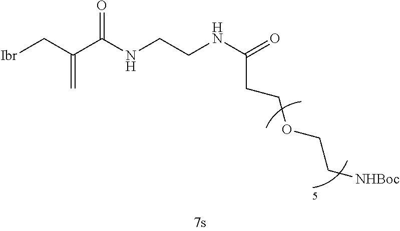 | PEGlylated boc amine |
| Afatinib | 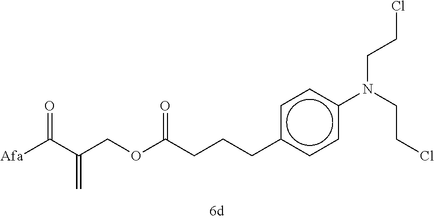 | Chlorambucil |
| Afatinib | 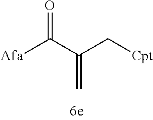 | Camptothecin |
| Afatinib | 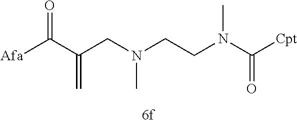 | Camptothecin |
| Afatinib | 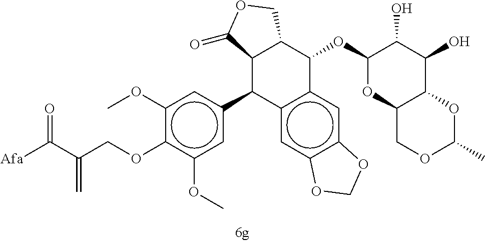 | Doxorubicine |
| Afatinib | 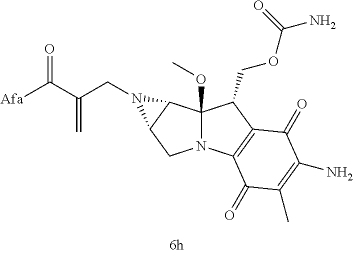 | Mytomycin-C |
| Afatinib | 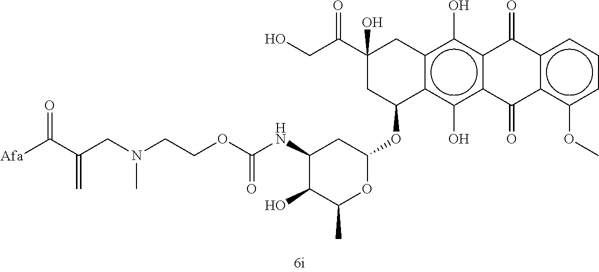 | Doxorubicine |
| Afatinib | 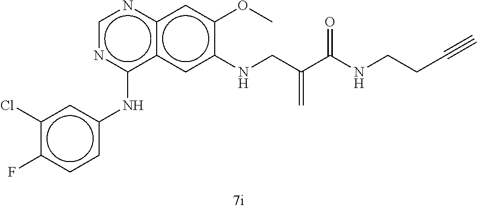 | alkyne |
| Afatinib | 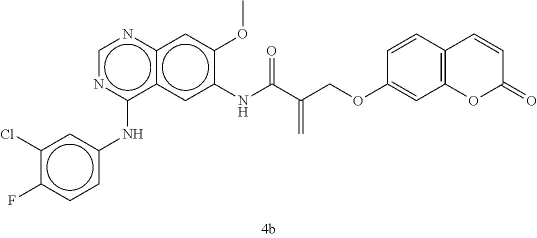 | coumarine |
| PL-pro inhibitor | 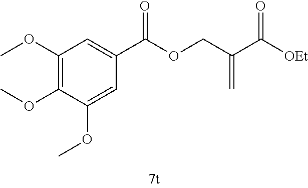 [SMILES: C=C(COC(=O)c1cc(OC)c(OC)c(OC)c1)C(=O)OCC] | Simple ester |
| Evotrutinib | 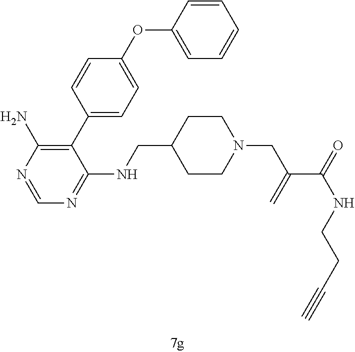 [SMILES: C#CCCNC(=O)C(=C)CN1CCC(CNc2ncnc(N)c2-c2ccc(Oc3ccccc3)cc2)CC1] | Alkyne |
| AMG-510 | 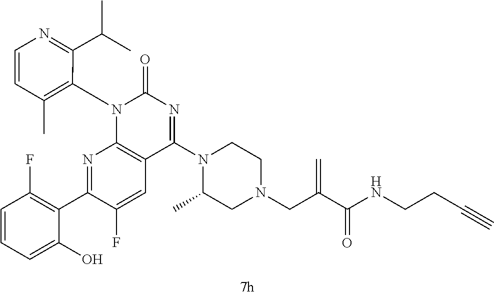 [SMILES: C#CCCNC(=O)C(=C)CN1CCN(c2nc(=O)n(-c3c(C)ccnc3C(C)C)c3nc(-c4c(O)cccc4F)c(F)cc23)[C@@H](C)C1] | alkyne |
| AMG-510 | 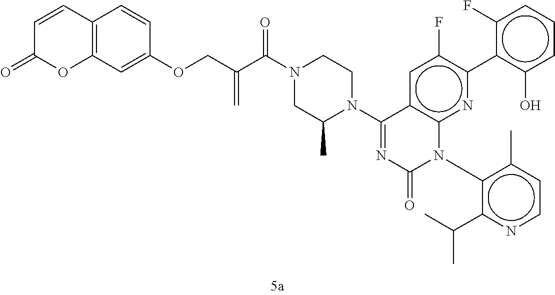 | coumarin |
| NEDD4 inhibitor | 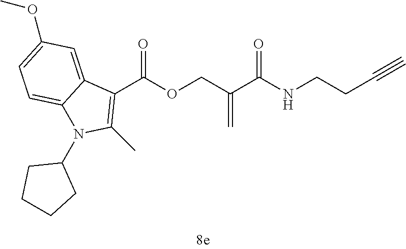 [SMILES: C#CCCNC(=O)C(=C)COC(=O)c1c(C)n(C2CCCC2)c2ccc(OC)cc12] | alkyne |
| NEDD4 inhibitor | 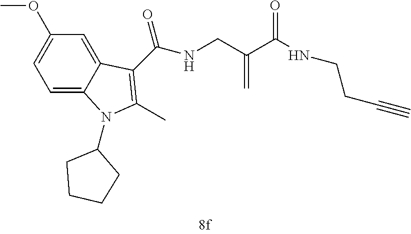 [SMILES: C#CCCNC(=O)C(=C)CNC(=O)c1c(C)n(C2CCCC2)c2ccc(OC)cc12] | alkyne |

α-substituted methacrylamides which upon reaction with thiol nucleophiles (See FIG. 1), undergo a conjugated addition-elimination reaction ultimately releasing the substituent at the alpha′ position. These compounds have been used as targeted covalent inhibitors and covalent ligand directed release (CoLDR) chemistry for the turn on fluorescence and chemiluminescence probes (FIG. 1). Several amines, phenols, carboxylic acids and carbamates successfully underwent elimination after the reaction with thiol group. The proper attachment of the ligand at alpha′ position of the methacrylamides can lead to the elimination of the ligand (recognition element) after the reaction with the functional group electrophile (i.e thiol of cysteine), which can be used for site-specific labeling at the protein active site with various probes (FIG. 2).

In some embodiment, this invention provides a protein proximity inducer of a first protein and a second protein comprising a Covalent Ligand Directed Releasing (CoLDR) Compound represented by the structures of Formula I, IA, IB or IC of this invention, wherein R is a protein binding ligand for a first protein and R₁ is another protein binding ligand for a second protein, wherein, upon interaction between the second protein and the corresponding protein binding ligand, R₁ is released, the second protein is then active and is labeled with R, inducing a new interaction with the first protein.

In another embodiment, a covalent bond is formed between the first protein and the corresponding protein binding ligand. In another embodiment, the covalent bond is formed via a nucleophilic moiety of protein A and the double bond (—C═CH₂) of the compounds of formula I, IA, IB or IC. In another embodiment, the nucleophilic moiety of the protein is a thiol, an amine, or a hydroxyl.

In some embodiments, the Covalent Ligand Directed Releasing (CoLDR) Compound of this invention is used as a protein labeling to diagnose a disease or a targeted protein. The labeling of a targeted protein is done by the changes in the fluorescence or chemiluminescence or radioactivity properties upon binding of the Covalent Ligand Directed Releasing (CoLDR) Compound of this invention to the targeted protein.

In some embodiments, the Covalent Ligand Directed Releasing (CoLDR) Compound of this invention is used as a protein sensor to diagnose a disease or a targeted protein. The sensing of a targeted protein is done by the changes in the fluorescence or chemiluminescence properties or radioactivity properties if a radiolabeled probe/radiopharmaceutical is used upon binding of the Covalent Ligand Directed Releasing (CoLDR) Compound of this invention to the targeted protein.

In some embodiments, the Covalent Ligand Directed Releasing (CoLDR) Compound of this invention is used as prodrug or a drug delivery system, wherein a drug is released upon binding of the Covalent Ligand Directed Releasing (CoLDR) Compound of this invention to a targeted protein.

In some embodiments, the Covalent Ligand Directed Releasing (CoLDR) Compound of this invention is used for protein proximity inducer wherein R of formula I, IA-IC is a protein binding ligand for the first protein and R₁ is another protein binding ligand for the second protein, wherein, upon interaction between the second protein and the its protein binding ligand, R₁ is released, and the second protein is then active and is labeled with R, inducing a new interaction with the first protein.

The prodrugs, drug delivery system, protein sensor, protein proximity inducer, or protein labeling of this invention offer several advantages for drug discovery and chemical biology including, predictable attenuation of reactivity, late-stage installation with no additional modifications to the core scaffold, and importantly the ability to functionalize compounds as turn-on probes.

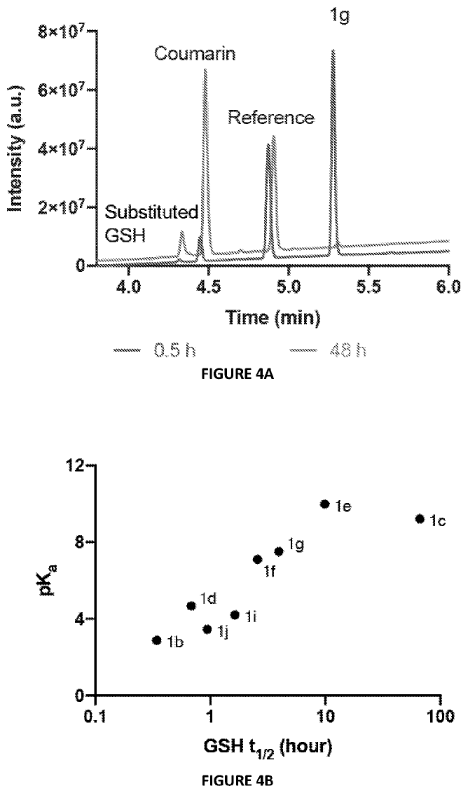

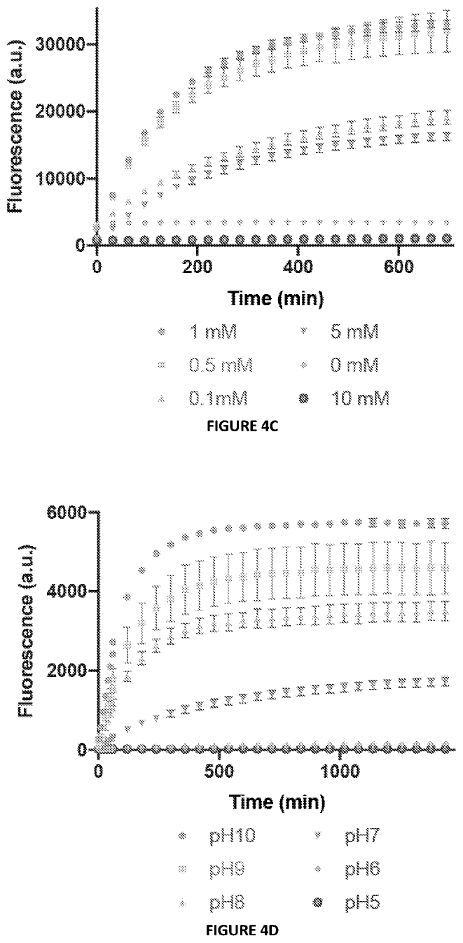

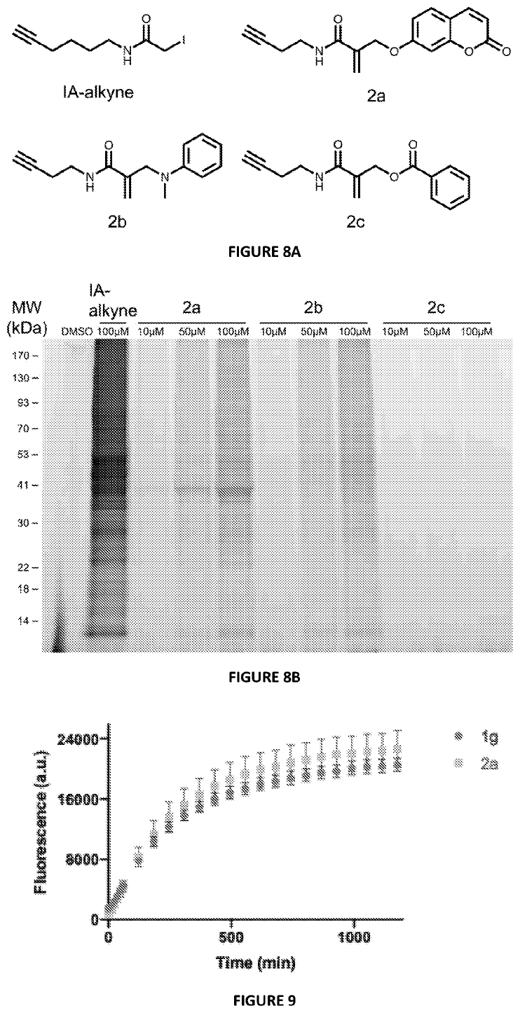

The substituted methacrylamides in the context of model compounds (See Example 2) span a wide window of thiol reactivity (as evaluated by t_{1/2} for their reaction with GSH; Table 1) which is predictable and depends on the pKₐ of their respective leaving group (FIG. 4B). These types of electrophiles are suitable for chemoproteomic applications with various proteomic reactivities (FIG. 8). As such these joins a growing collection of cellular compatible, cysteine targeting electrophiles that may expand the scope of the targetable cysteinome. These methacrylamides leave an identical adduct on proteomically labelled cysteines, mixtures of such compounds may be used as convenient probes for quantitative chemoproteomics with potentially increased coverage.

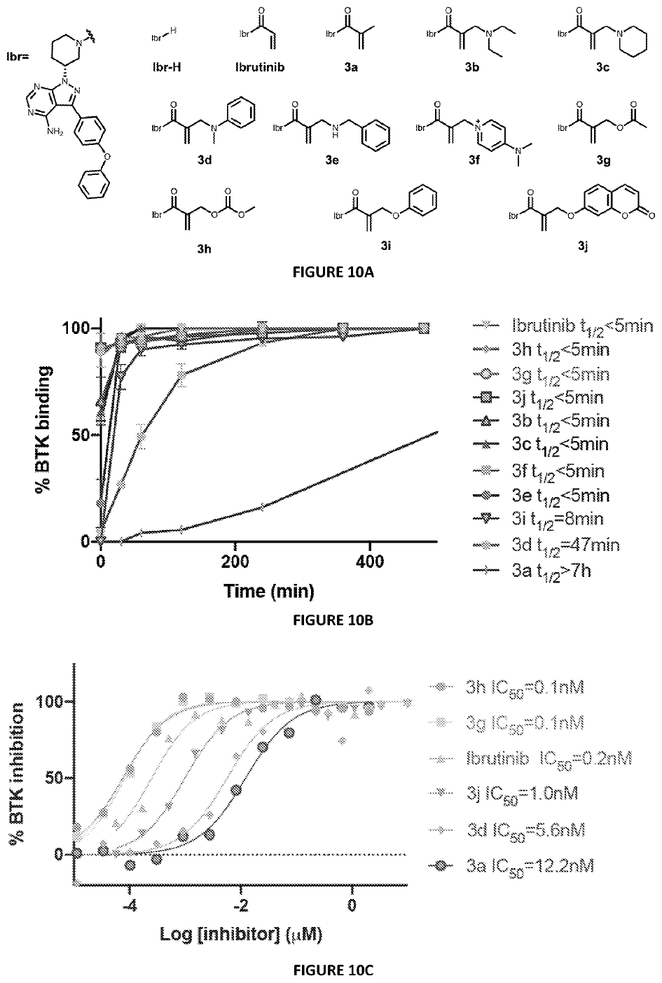

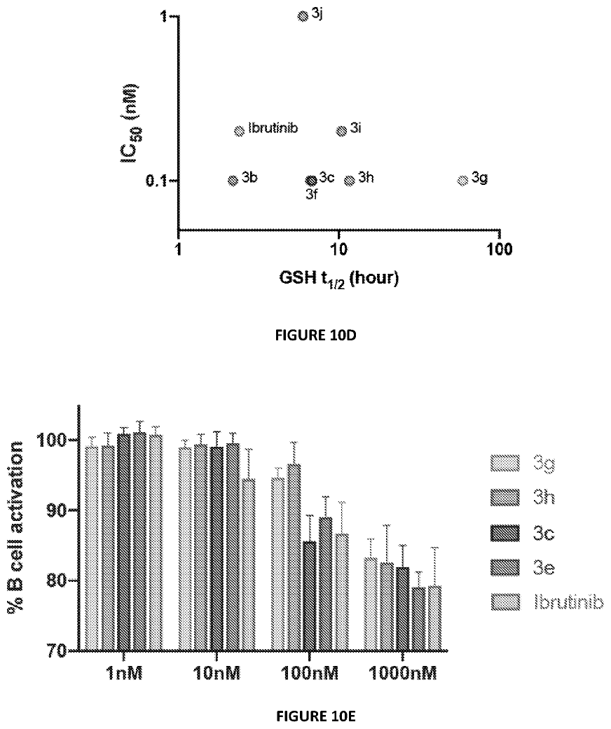

In the context of targeted covalent inhibitors, the model compounds (See Example 2) demonstrated significantly reduced thiol reactivity (FIG. 10C) and the vast majority of compounds showed lower GSH reactivity than the parent unsubstituted acrylamide. This may confer improved selectivity for such targeted covalent inhibitors, by lowering the number of possible off-targets as was previously shown for lower-reactivity covalent analogs of Ibrutinib. In this context, it is also interesting to note the cellular reactivity of the ester probes (e.g. 2c and 3g) which may also confer kinetic selectivity as was previously shown for fumarate esters.

Several of these compounds showed improved inhibition of BTK over Ibrutinib, which is already a highly optimized BTK inhibitor (FIG. 10B-E). Perhaps through locking the electrophile in a conformation more compatible with covalent bond formation. This suggests that this class of electrophiles can be useful for late-stage optimization of targeted covalent inhibitors. Particularly since they can be installed directly on the acrylamide. Functional assays for B cell receptor signaling inhibition, in primary B cells, showed that they are active in a cellular context with comparable potency to Ibrutinib (FIG. 10E).

In some embodiments, this new class of electrophiles provides the ability to trigger the release of a chemical cargo, facilitated by a specific target cysteine. Most of the previously reported turn-on approaches are based on enzymatic functions by reductases, glycosidases, proteases, and lactamases. In the context of covalent labeling, acyloxymethyl ketones were used to generate FRET-based turn-on fluorescent probes for proteases, quinone methide chemistry was also used for quenched activity-based probes. Recently, PET-based and cysteine reactive turn-on fluorescent probes have also been reported. Relatedly, Hamachi and colleagues reported several ligand directed chemistries, in which a guiding ligand leaves the active site after the probe reacts with random nucleophilic residues (lysine, serine, and histidine) on the protein surface. These methods have been used to develop turn-on fluorescent probes (32-36), but require the ligand to retain high affinity and selectivity towards its target protein after modification with relatively large reactive groups.

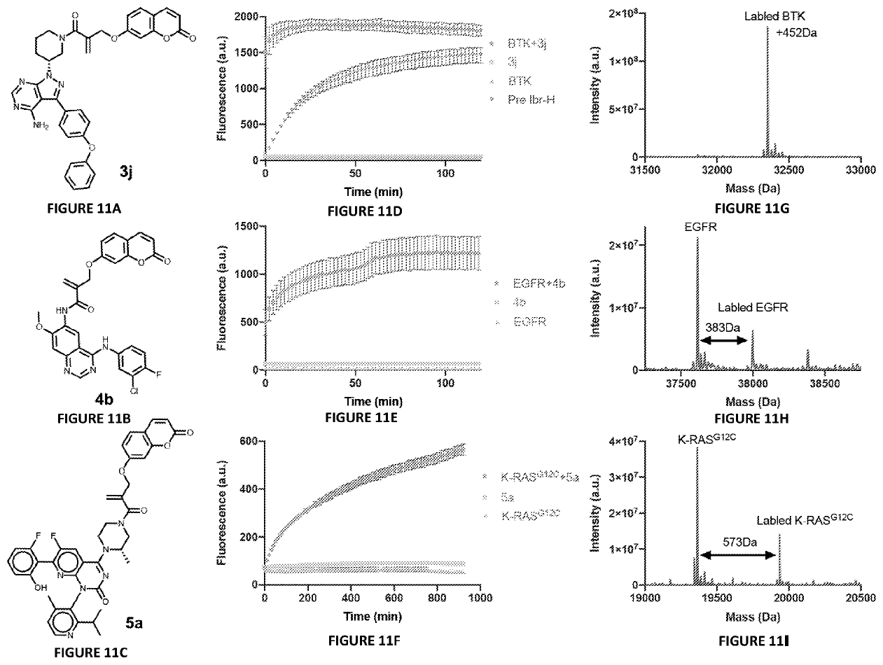

In this invention, the turn-on release of a fluorophore is triggered, in a selective fashion (FIG. 11; FIG. 13). The approach is demonstrated generally, coined as CoLDR chemistry, by applying it to three various targeted covalent inhibitors, including against the challenging K-Ras^{G12C} oncogenic mutant. This approach is of course not limited to fluorophores. Since there is a wide scope of compatible leaving group functionalities (phenols, amines, carboxylic acids) many cargoes should be available for targeted release such as pro-drugs (37-39), chemotherapeutic agents (40-41), imaging agents (42-44), or self immolative linkers (16) potentially useful for both diagnostics as well as therapeutics.

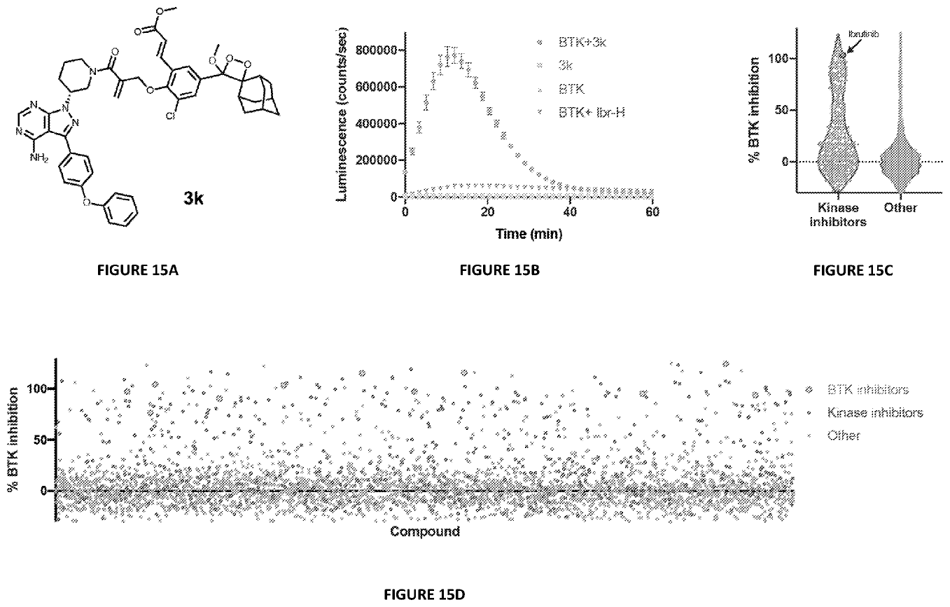

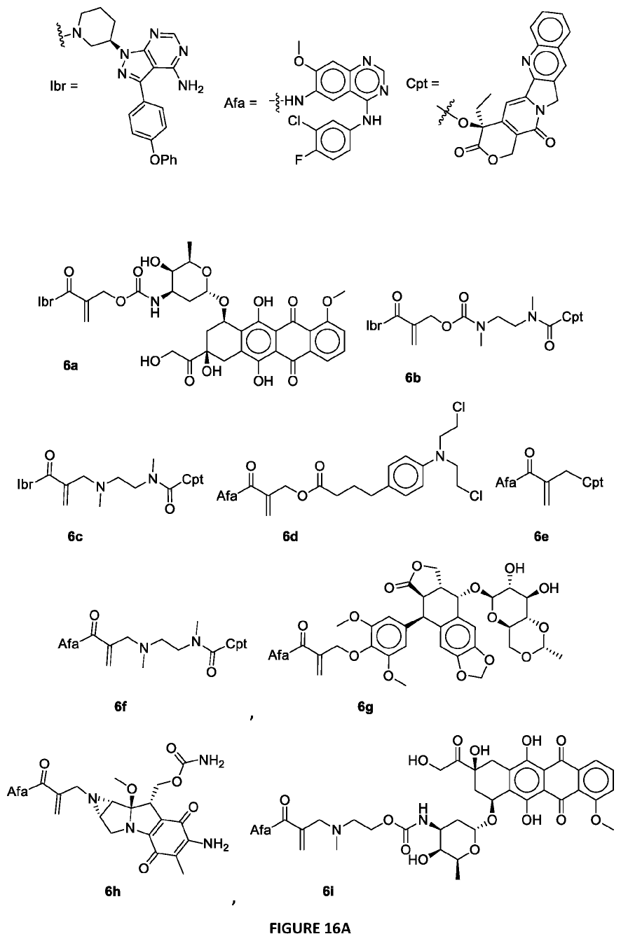

In this invention, it is demonstrated that CoLDR chemistry is also applicable for the generation of turn-on chemiluminescence (FIG. 15) and has used this novel functional probe to facilitate a small high-throughput screen against BTK resulting in the identification of known BTK inhibitors and non-selective kinase inhibitors. This assay is considerably simpler than typical enzymatic based assay, as it does not require any substrate or enzymatic reaction optimization. Moreover, it has the benefit of site-selective screening, since only inhibitors that will compete with the probe binding next to its target will reduce the signal. A similar screen with the K-Ras^{G12C} probe for instance is expected to identify mainly switch-II pocket binders. This allows a convenient method to screen e.g., for allosteric binders is present near the target pocket.

The Covalent Ligand Directed Releasing (CoLDR) Compound structures of this invention can be used to modulate the reactivity of selective covalent inhibitors or can be used as turn-on fluorogenic probes against proteins (such as BTK, EGFR, and K-Ras^{G12C}), and with a turn-on chemiluminescent probe for BTK.

In this invention the α-substituted methacrylamides of the structures of Formula I, IA, IB or IC are new class of electrophiles suitable for targeted covalent inhibitors. While typically α-substitutions inactivate acrylamides, hetero α-substituted methacrylamides are showing to have higher nucleophilic reactivity with the protein and undergo a conjugated addition-elimination reaction ultimately releasing the substituent. Their nucleophilic reactivity with the protein is tunable and correlates with the pKₐ of the leaving group.

Using the covalent ligand directed release (CoLDR) chemistry provided herein, various potential drug targets like BTK, KRAS, SARS-Cov-2-PLpro were modified with different probes. For BTK selective labelling in cells were shown of both alkyne and fluorophores tags. Protein labelling by traditional affinity methods often inhibits protein activity since the directing ligand permanently occupies the target binding pocket. Using CoLDR chemistry, modification of BTK by the probes provided herein in cells preserves its activity. Further, the half-life of drug targets (such as BTK) in its native environment with minimal perturbation is being determined using the Covalent Ligand Directed Releasing (CoLDR) Compound structures of this invention. Using an environment-sensitive ‘turn-on’ fluorescent probe, the ligand binding to the active site of drug targets (such as BTK) is monitored. In another embodiment the efficient degradation of BTK by CoLDR-based BTK PROTACs (DC₅₀<100 nM), which installed a E3 ligase binder target (e.g. CRBN binder) onto BTK is provided. In another embodiment provided herein an efficient degradation of a protein target by CoLDR-based PROTACs are provided by installing an E3 ligase binder covalently on the target. This type of Proteolysis targeting chimeras (PROTACs) may enable the tuning of degradation kinetics of the target protein while keeping the protein in its active form. This approach joins very few available labeling strategies that maintain the target protein activity and thus makes an important addition to the toolbox of chemical biology.

In some embodiments, the compounds or probes disclosed herein are used to label proteins (non-limiting examples include: BTK, KRAS, and SARS-COV-2-PLpro) to their active site (having hydroxyl, thiol or amine groups). This site-selective labeling comes with many advantages like the development of “turn on” fluorescent probes, half-life identification in the native cellular environment, and PROTACs (Proteolysis targeting chimeras) for degradation.

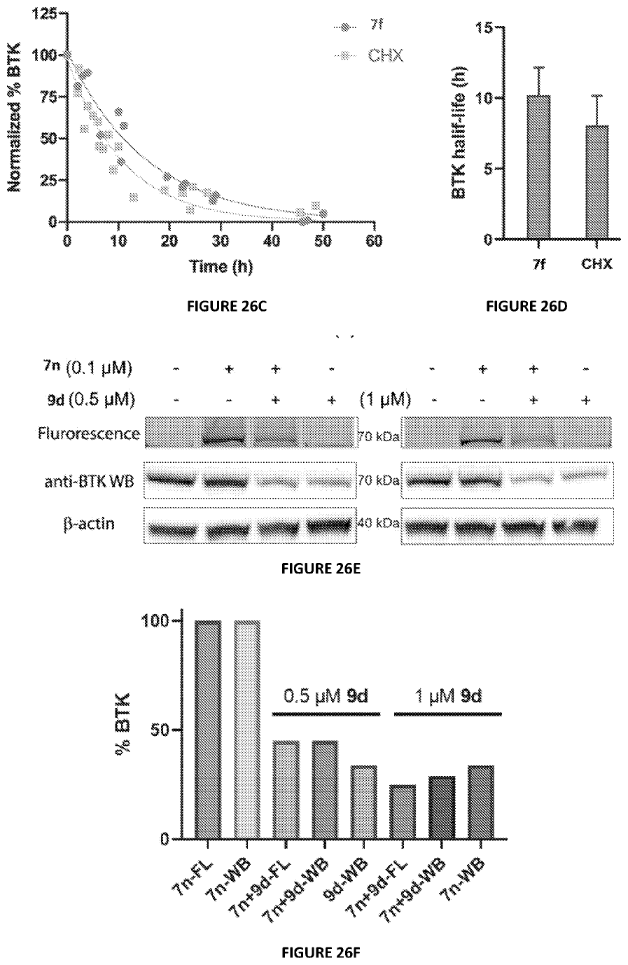

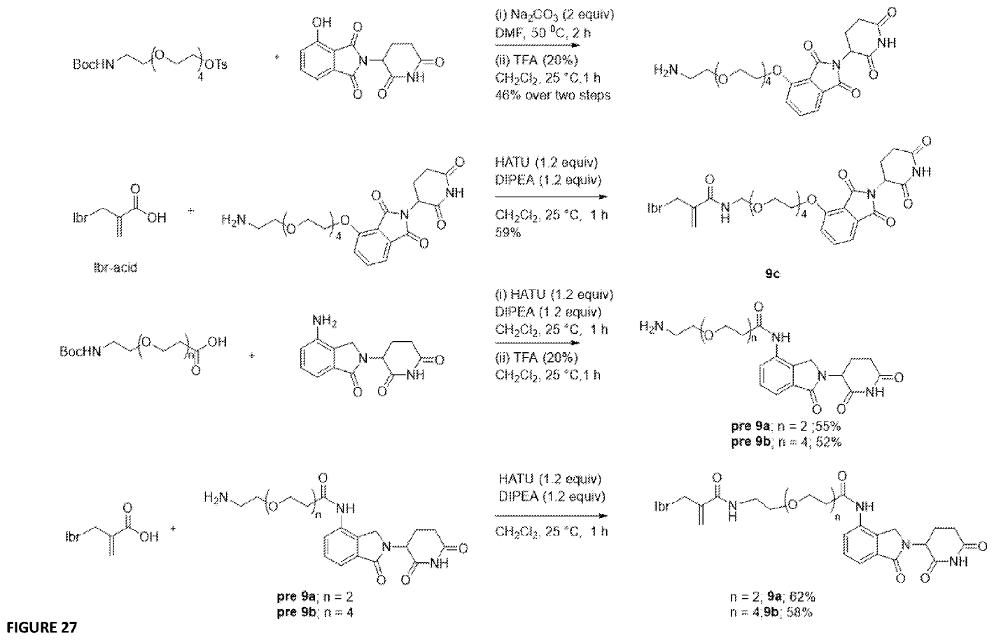

In some embodiments, the compounds/probes disclosed herein are used for ligand-directed chemistry—for the identification of off-targets of potential covalent inhibitors or for imaging experiments. As these compounds are derived from their corresponding covalent inhibitors, no optimization of linker length is required to label the same functional group (i.e thiol of the cysteine). The importance of these probes is that they don't inhibit the activity of the native protein and their downstream signals after labeling with activity probes (FIG. 26). This allows to study the properties of the protein in the native cellular environment.

In some embodiments, the compounds/probes disclosed herein are used for labeling an environmentally sensitive dye (i.e. Nile red) to a protein (i.e. BTK) as a turn-on fluorescent probe, which shows an improvement in the fluorescent intensity. Since environmental sensitive probes give information of the protein structure, and the presence of ligands could change its structure, this method helps to find the structure of the protein in the absence of the ligand. Further, the lack of ligand in the active site keeps the protein active with turn-on fluorescence.

In some embodiments, the compounds/probes disclosed herein are used to find the half-life of a protein in its native cellular environment without interfering with the other biological processes. Several methods like pulse-chase radiolabeling assay and cycloheximide (CHX) assay for the identification of half-life of the protein have been reported. The main disadvantage of the pulse-chase assay is that it includes many steps that can be time-consuming and requires radiolabeling. Furthermore, cycloheximide changes the cellular process by stopping the synthesis of all the proteins. The compounds/probes disclosed herein do not change half-life in cycloheximide assay whereas Ibrutinib reduces its half-life by two hours. The modification of protein half life without affecting its activity may be possible with different functional moieties like PEG linkers, or hydrophobic degraders.

In some embodiments, the compounds/probes disclosed herein are used for the degradation of a protein (i.e BTK) using PROTACs, wherein the covalently attached E3 ligase binder (i.e. CRBN binder) to the protein without the ligand degrades it efficiently. This method could help to tune the protein degradation kinetics without affecting its activity.

In some embodiments, provided herein CoLDR Compounds of formula I, IA-IC wherein R or R₁ are both protein binding ligands and one of R or R¹ is an Ubiquitin ligase binder, thereby obtaining a CoLDR-based protein PROTAC compound.

In some embodiments, the compounds/probes disclosed herein are used for labeling proteins in native cellular environment which upon labeling releases the ligand thereby stays active. This method enables various applications like half-life identification and targeted degradation of proteins.

In some embodiments, the compounds/probes disclosed herein allow the site-specific cellular labeling of a native protein of interest while sparing its enzymatic activity.

The advantage of the compounds/probes disclosed herein is that there is no need to change the position of the electrophilic carbon, minimizing the risk of interfering with covalent bond formation to the target. It also means that it is known a priori which residue will be labeled with the newly installed tag.

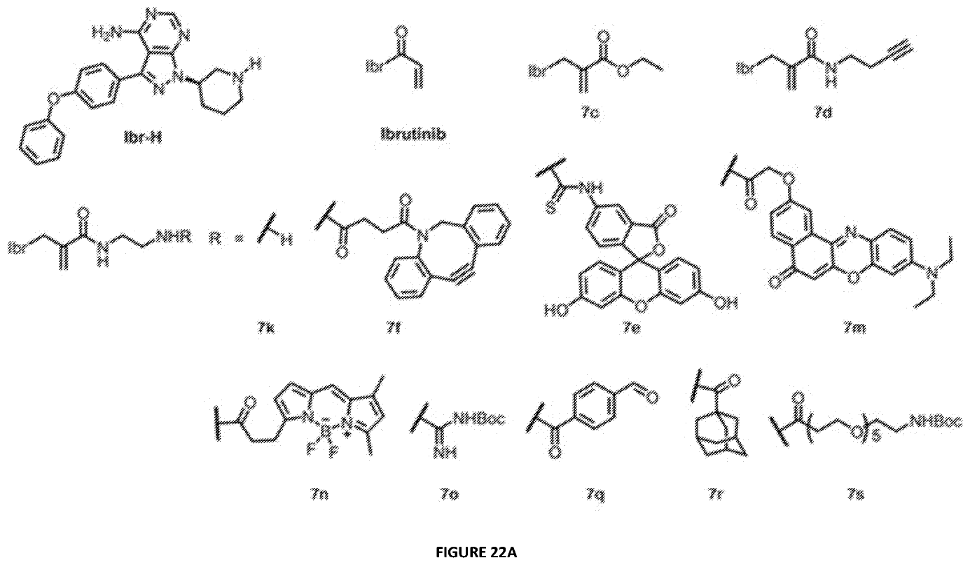

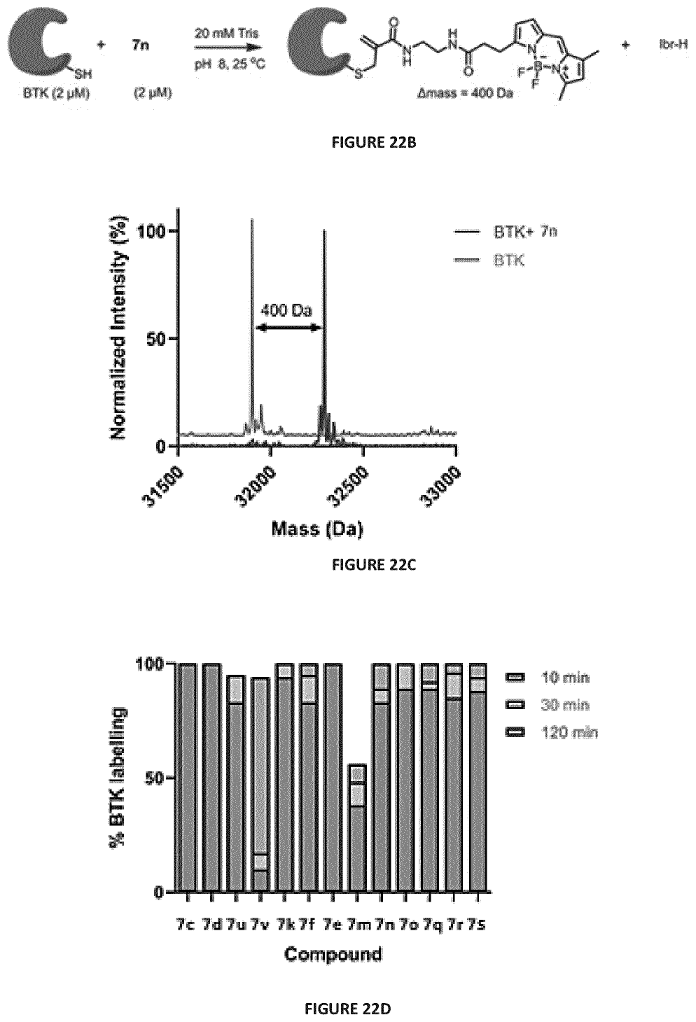

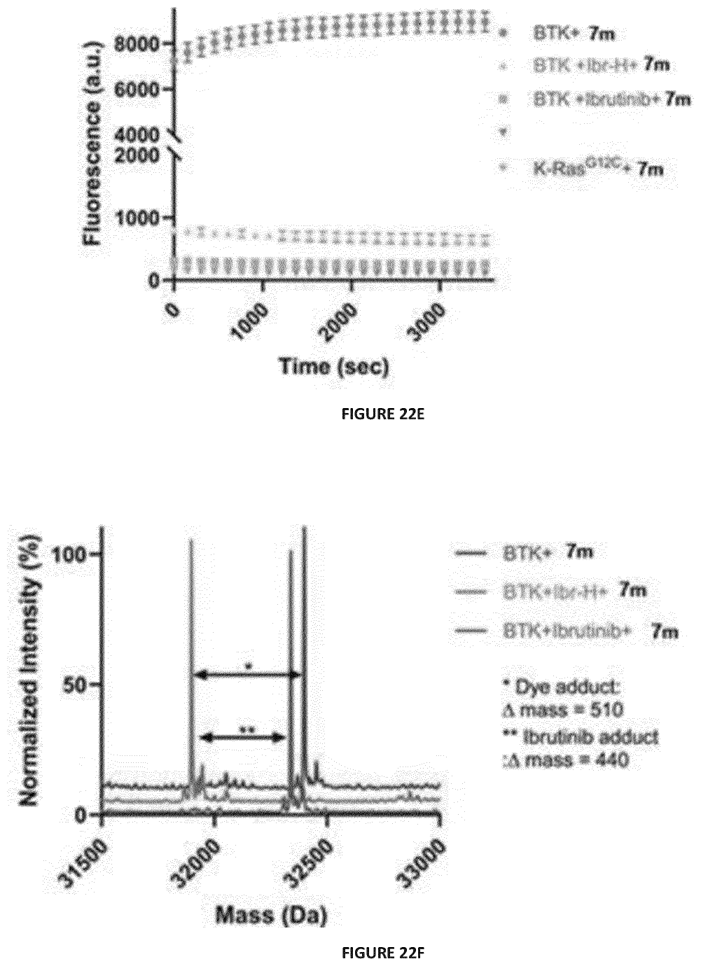

It has been shown that tags with a wide variety of functionalities could be installed (FIG. 22A), indicating that the approach is versatile.

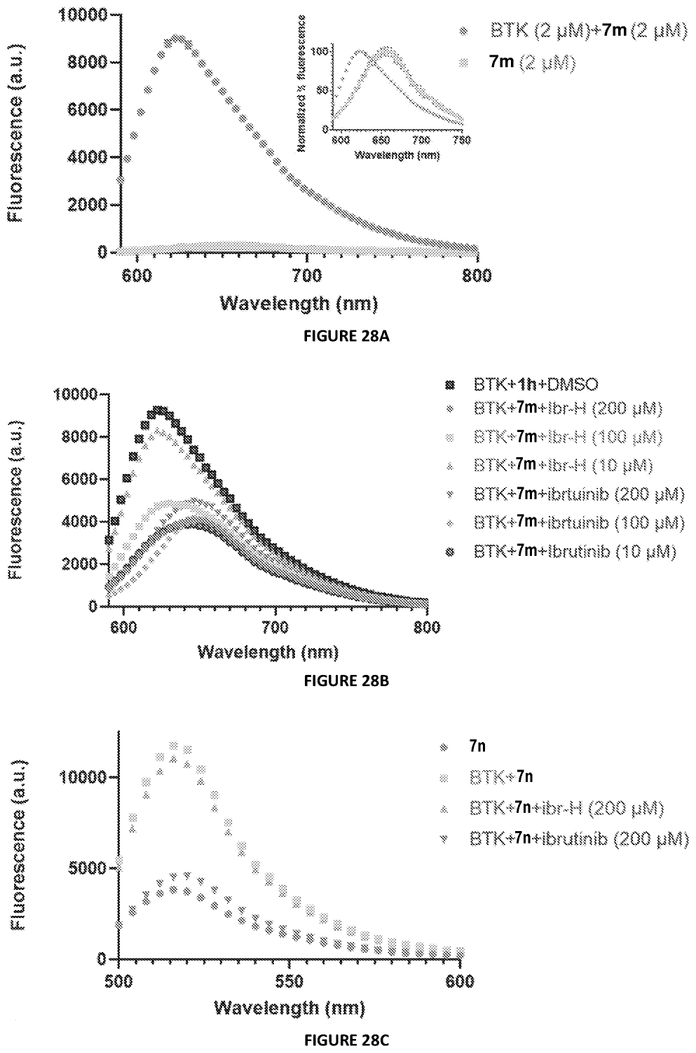

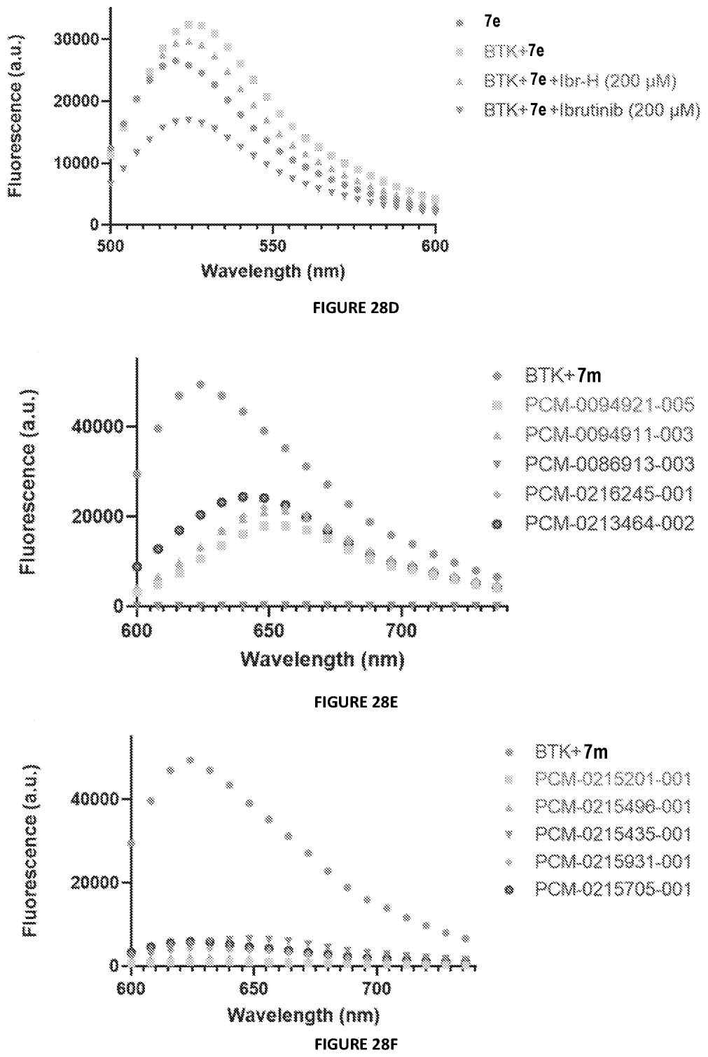

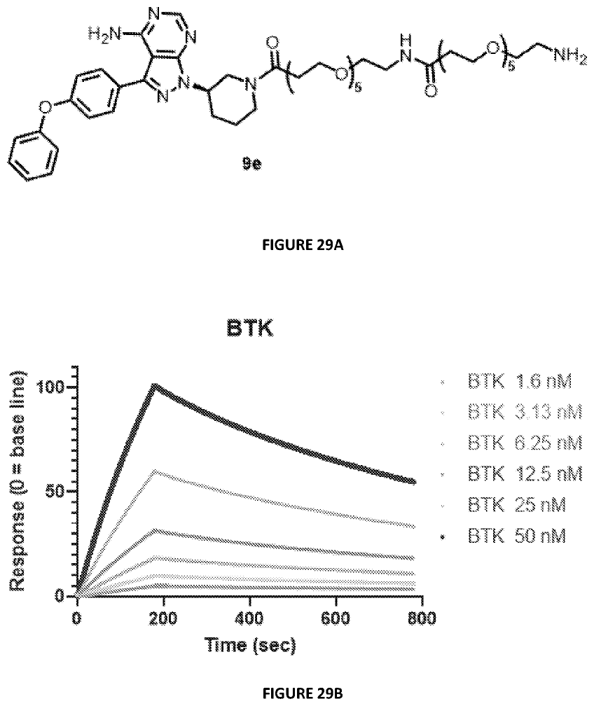

In some embodiments, the use of the compounds/probes disclosed herein for labeling platform provides an environment-sensitive ‘turn-on’ fluorescent probe. In addition to the generation of fluorescence upon binding, the active protein is labeled, and the dye can serve as a reporter for binding events in the protein (FIG. 28) and perhaps for its conformation. The fact that probes provided herein do not hinder binding to the active site, can facilitate investigation of alternative ligands binding events.

Provided herein, a new platform for site-specific labeling of proteins, that is compatible with cellular conditions and spares the labeled protein's activity. This approach joins very few such available strategies and thus makes an important addition to the toolbox of chemical biology.

As used herein, the term alkyl, used alone or as part of another group, refers, in one embodiment, to a “C1 to C18 alkyl” and denotes linear and branched, saturated or unsaturated (e.g., alkenyl, alkynyl) groups, the latter only when the number of carbon atoms in the alkyl chain is greater than or equal to two, and can contain mixed structures. Non-limiting examples are alkyl groups having from 1 to 6 carbon atoms (C1 to C6 alkyls), or alkyl groups having from 1 to 4 carbon atoms (C1 to C4 alkyls). Examples of saturated alkyl groups include, but are not limited to, methyl, ethyl, n-propyl, isopropyl, n-butyl, iso-butyl, sec-butyl, tert-butyl, amyl, tert-amyl and hexyl. Examples of alkenyl groups include, but are not limited to, vinyl, allyl, butenyl and the like. Examples of alkynyl groups include, but are not limited to, ethynyl, propynyl and the like. Similarly, the term “C1 to C18 alkylene” denotes a bivalent radical of 1 to 18 carbons.

The alkyl group can be unsubstituted, or substituted with one or more substituents selected from the group consisting of halogen, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, or alkylsulfonyl groups. Any substituents can be unsubstituted or further substituted with any one of these aforementioned substituents.

The term “aryl” used herein alone or as part of another group denotes an aromatic ring system having from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. The aryl group can be unsubstituted or substituted through available carbon atoms with one or more groups such as halogen, alkyl, aryl, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, alkylsulfonyl —OCN, —SCN, —N═C═O, —NCS, —NO, —N3, —OP(═O)(OR*)2, —P(═O)(OR*)2, —P(═O)(O-)2, —P(═O)(OH)2, —P(O)(OR*)(O—), —C(═O)R*, —C(═O)X, —C(S)R*, —C(S)OR*, —C(O)SR*, C(S)SR*, —C(S)NR* 2 or —C(═NR*)NR* 2 groups, where each R* is independently H, alkyl, aryl, arylalkyl, a heterocycle, or a protecting group or prodrug moiety groups. Any substituents can be unsubstituted or further substituted with any one of these aforementioned substituents.

The term “heteroaryl” refers to an aromatic ring system containing from 5-14 member ring having at least one heteroatom in the ring. Non-limiting examples of suitable heteroatoms which can be included in the aromatic ring include oxygen, sulfur, phospates and nitrogen. Non-limiting examples of heteroaryl rings include pyridinyl, pyrrolyl, oxazolyl, indolyl, isoindolyl, purinyl, furanyl, thienyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, etc. The heteroaryl group can be unsubstituted or substituted through available carbon atoms with one or more groups such as. halogen, alkyl, aryl, hydroxy, alkoxy, aryloxy, alkylaryloxy, heteroaryloxy, oxo, cycloalkyl, phenyl, heteroaryls, heterocyclyl, naphthyl, amino, amido, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, alkylsulfonyl, —OCN, —SCN, —N═C═O, —NCS, —NO, —N3, —OP(═O)(OR*)2, —P(═O)(OR*)2, —P(═O)(O-)2, —P(═O)(OH)2, —P(O)(OR*)(O—), —C(═O)R*, —C(═O)X, —C(S)R*, —C(S)OR*, —C(O)SR*, C(S)SR*, —C(S)NR* 2 or —C(═NR*)NR* 2 groups, where each R* is independently H, alkyl, aryl, arylalkyl, a heterocycle, or a protecting group or prodrug moiety. Any substituents can be unsubstituted or further substituted with any one of these aforementioned substituents.

The terms “compound” and “probe” are used herein interchangeably.

### EXAMPLES

### Methods

### LC/MS Measurements

LC/MS runs were performed on a Waters ACQUITY UPLC class H instrument, in positive ion mode using electrospray ionization. UPLC separation for small molecules used a C18-CSH column (1.7 μm, 2.1 mm×50 mm). The column was held at 40° C. and the autosampler at 10° C. Mobile phase A was 0.1% formic acid in the water, and mobile phase B was 0.1% formic acid in acetonitrile. The run flow was 0.3 mL/min. The gradient used was 100% A for 2 min, increasing linearly to 90% B for 5 min, holding at 90% B for 1 min, changing to 0% B in 0.1 min, and holding at 0% for 1.9 min (For 1b, the gradient started from 100% A and decreasing linearly to 60% A for 2 min, 60%-40% A for 2.0-6.0 min, 40%-10% A in 0.5 min, and 10%-100% A for 1.5 min). UPLC separation for proteins used a C4 column (300 Å, 1.7 μm, 2.1 mm×100 mm). The column was held at 40° C. and the autosampler at 10° C. Mobile solution A was 0.1% formic acid in the water, and mobile phase B was 0.1% formic acid in acetonitrile. The run flow was 0.4 mL/min with gradient 20% B for 2 min, increasing linearly to 60% B for 3 min, holding at 60% B for 1.5 min, changing to 0% B in 0.1 min, and holding at 0% for 1.4 min (For the kinetic labeling experiment, the gradient used was 90% A for 0.5 min, 90-40% A for 0.50-2.30 min, 40-10% A for 2.60-3.20 min, 10% A for 0.2 min, 10-90% A for another 0.2 min and 90% A for 0.6 min. The mass data were collected on a Waters SQD2 detector with an m/z range of 2-3071.98 at a range of m/z of 800-1500 Da for BTK, 900-1800 Da for EFGR, and 750-1550 for K-RAS^{G12C}.

### MS/MS Based Proteomics

of 5 μM Recombinant BTK kinase domain was incubated in 20 mM Tris with 50 μM of 7d or DMSO. The compounds were then removed by methanol-chloroform (400 μL MeOH+100 μL CHCl₃+300 μL H₂O) precipitation of the protein. The dry pellet was dissolved in 50 μl of 50 mM Tris pH 8+5% SDS and heated to 95° C. for 6 min. The concentration of the protein was estimated using BCA assay (using BSA as the standard). 2 μg each sample were diluted to 15 μl with Tris 50 mM pH=8+5% SDS, reduced with DTT (0.75 μl of 0.1 M in 5% SDS/Tris 50 mM pH 8, 45 min 65° C.), cooled to room temperature, then alkylated with 0.75 μl of 0.2 M iodoacetamide in water (30 min room temperature in the dark). The protein was then isolated and trypsinized on s-traps (Protifi) according to the manufacturer's instructions. Triplicates were prepared for each molecule.

ULC/MS grade solvents were used for all chromatographic steps. Each sample was loaded using split-less nano-Ultra Performance Liquid Chromatography (10 kpsi nanoAcquity; Waters, Milford, MA, USA). The mobile phase was: A) H₂O+0.1% formic acid and B) acetonitrile+0.1% formic acid. Desalting of the samples was performed online using a reversed-phase Symmetry C18 trapping column (180 μm internal diameter, 20 mm length, 5 μm particle size; Waters). The peptides were then separated using a T3 HSS nano-column (75 μm internal diameter, 250 mm length, 1.8 μm particle size; Waters) at 0.35 μL/min. Peptides were eluted from the column into the mass spectrometer using the following gradient: 4% to 30% B in 155 min, 35% to 90% B in 5 min, maintained at 90% for 5 min and then back to initial conditions.

The nanoUPLC was coupled online through a nanoESI emitter (10 m tip; New Objective; Woburn, MA, USA) to a quadrupole orbitrap mass spectrometer (Q Exactive HFX, Thermo Scientific) using a FlexIon nanospray apparatus (Proxeon).

Data was acquired in data dependent acquisition (DDA) mode, using a Top10 method. MS1 resolution was set to 120,000 (at 200 m/z), mass range of 375-1650 m/z, AGC of 3e6 and maximum injection time was set to 60 msec. MS2 resolution was set to 15,000, quadrupole isolation 1.7 m/z, AGC of 1e5, dynamic exclusion of 45 sec and maximum injection time of 60 msec.

### Proteomics Analysis

Analysis was done using MaxQuant 1.6.3.4. The sequence of BTK was used for the analysis. The digestion enzyme was set to Trypsin with a maximum number of missed cleavages of 0. Carbamidomethyl and the modification by the molecule were included as variable modifications on cysteine. The “Re-quantify” option was enabled. Contaminants were included. Peptides were searched with a minimum peptide length of 7 and a maximum peptide mass of 4,500 Da. “Second peptides” were enabled and “Dependent peptides” were disabled. The option “Match between runs” was enabled with a Match time window of 0.7 min and an alignment window of 20 min. An FDR of 0.01 was used for Protein FDR, PSM FDR and XPSM FDR. The triplicate measured for each compound (or for DMSO-treated protein) was analyzed separately.

Following MaxQuant analysis, only fully cleaved peptides were quantified and cysteine-containing peptides that were not modified by either iodoacetamide or compound were ignored. The intensity for each peptide was calculated as the average of the three triplicates. If the intensity was zero for one of the replicates the peptide was ignored. The intensities for the non-cysteine containing peptides were averaged for each data set and used to normalize the intensity of cysteine containing peptides. Estimation of the extent of labeling of cysteine-containing peptides in the sequence was done by comparing the intensity of carbamidomethyl-modified peptides between the DMSO and molecule-treated samples. MS/MS spectra for the carbamidomethyl-modified and molecule modified peptides were extracted using Skyline.

Labeling Experiments of Ibrutinib Derivatives with BTK

BTK kinase domain was expressed and purified as previously reported (46). Binding experiments were performed in Tris 20 mM pH 8.0, 50 mM NaCl at room temperature. The BTK kinase domain was diluted to 2 μM in the buffer, and 2 μM Ibrutinib derivatives (7c, 7d, 7k, 7f, 7e, 7m, 7n, 7o, 7q, 7r, 7s, and 7g) were added by adding 1/100th volume from a 200 μM solution. The reaction mixtures, at room temperature for various times, were injected into the LC/MS. For data analysis, the raw spectra were deconvoluted using a 20000:40000 Da window and 1 Da resolution. The labeling percentage for a compound was determined as the labeling of a specific compound (alone or together with other compounds) divided by the overall detected protein species. For K-Ras^{G12C} 10 μM of protein was incubated with 100 μM of compound 7h in Tris 20 mM pH 8.0, 50 mM NaCl at 37° C. for 16 h. For PLpro, 2 μM of protein was incubated with 10 μM 7t in 300 mM NaCl, 50 mM Tris pH 8, 1 mM TCEP at 25° C. for 16 h.

### Plate Reader Fluorescence and Luminescence Measurements

Plate reader measurements were performed on Tecan Spark Control 10M fluorescent measurements using black 384 well plates with clear bottom. Luminescence measurements were performed using 384 white well plates, Integration for 100 ms and 1 ms settle time.

Fluorescence Intensity Measurements with 7m

The BTK kinase domain was diluted to 2 μM in the buffer, and 2 μM 7m was added by adding 1/100th volume from a 200 μM solution. Control measurements were performed without protein and BTK with preincubation with 4 μM Ibr-H/Ibrutinib for 5 min. Each condition was done in quadruplicate in 20 mM Tris pH 8.0 and 50 mM NaCl for BTK. Fluorescent measurements were taken every 2 min for 1 h for BTK/K-Ras^{G12C}. At the end of the measurements, samples were injected directly into the LC/MS for labeling quantification.

High Throughput Screening with 7m

High-throughput screening was performed with the Selleck compound collection at 200 μM for the initial screen in 384-well black plates (Thermo Fisher Scientific-Nunclon 384 Flat Black [NUN384fb]). BTK (2 μM) was incubated with compound 7m (4 μM) for 1 h. The resulting BTK-7m (50 μL) was added to the inhibitors. The screen was performed with 20 mM Tris pH 8.0, 50 mM NaCl at 32° C. and fluorescence was recorded after 10 min.

### GSH Reactivity Assay for Ibrutinib Derivatives

A 100 μM (0.5 μL of 20 mM stock) sample of the electrophile (7c, 7d, 7k, 7f, 7e, 7m, 7n, 7o, 7q, 7r, 7s) was incubated with 5 mM GSH (5 μL of 100 mM stock, freshly dissolved), 5 mM NaOH (to counter the acidity imparted by GSH) and 100 μM 4-nitrocyano benzene (0.5 μl of 20 mM stock solution) as an internal standard in 100 mM potassium phosphate buffer pH 8.0 and DMF at a ratio of 9:1, respectively. All solvents were bubbled with argon. Reaction mixtures were kept at 10° C. Every 1 h 5 μL from the reaction mixture were injected into the LC/MS. The reaction was followed by the peak area of the electrophile normalized by the area of the 4-nitrocyano benzene (i.e. by the disappearance of the starting material). The natural logarithm of the results was fitted to linear regression, and t½ was calculated as t½=ln 2/−slope.

### GSH Reactivity Assay for Model Compounds

A 100 μM (5 μL of 20 mM stock) sample of the electrophile (1a-1j) was incubated with 5 mM GSH (50 μL of 100 mM stock) and 100 μM 4-nitrocyano benzene (5 μl of 20 mM stock solution) as an internal standard in 100 mM potassium phosphate buffer of pH 8.0 (940 μL), respectively. All solvents were bubbled with argon. Reaction mixtures were kept at 37° C. with shaking. After certain intervals of time as shown in the graph, 5 μL from the reaction mixture was immediately injected into the LC/MS. The reaction was followed by the peak area of the electrophile normalized by the area of the 4-nitrocyano benzene. Natural logarithms of the results were fitted to linear regression, and t_{1/2} was calculated as t_{1/2}=ln 2/−slope.

### GSH Reactivity of 1g Measured Via Fluorescence.

A 100 μM of 1g was added separately to 0.1, 0.5, 1, 5 and 10 mM GSH in 100 mM potassium phosphate buffer pH 8.0. Immediately fluorescence intensity measurements at 435 nm at 37° C. were acquired every 10 min for 1 h and every 1 h for 24 h. The assay was performed in a 384-well plate using a Tecan Spark10M plate reader. Control experiments without GSH and 1g were also conducted. Compounds were measured in triplicate.

Effect of pH on the Reactivity of 1g with GSH

A 100 μM of Ig was added 5 mM GSH in 100 mM potassium phosphate buffer of various pH 5.0, 6.0, 7.0, 8.0. 9.0 and 10.0. Immediately fluorescence intensity measurements at 435 nm at 37° C. were acquired every 10 min for 1 h and every 1 h for 24 h. The assay was performed in a 384-well plate using a Tecan Spark10 M plate reader. Compounds were measured in triplicate.

### GSH Reactivity Assay for Ibrutinib Derivatives

A 100 μM of the electrophile (3a-3l) was incubated with 100 μM, 4-nitrocyano benzene as internal standard, and 5 mM GSH in 100 mM potassium phosphate buffer pH 8.0 (titrated after the addition of GSH) and DMF at a ratio of 9:1, respectively. All solvents were bubbled with argon. Reaction mixtures were kept at 37° C. with shaking. After certain intervals of time as shown in the graph (1.5 h, 4 h, 8 h, 12 h, 24 h, 48 h, 72 h), 50 μL from the reaction mixture was immediately injected into the LC/MS. The reaction was followed by the peak area of the electrophile normalized by the area of the 4-nitrocyano benzene. Natural logarithms of the results were fitted to linear regression, and t_{1/2} was calculated as t_{1/2}=ln 2/−slope.

Kinetic Labeling Experiments of Ibrutinib Derivatives with BTK

BTK kinase domain was expressed and purified as previously reported⁶⁵. Binding experiments were performed in Tris 20 mM pH=8, 50 mM NaCl, and 1 mM DTT. BTK kinase domain was diluted to 2 μM in the buffer, and 2 μM Ibrutinib derivatives were added by adding 1/100th volume from a 200 μM solution. The reaction mixtures, at room temperature for various times, were injected into the LC/MS. For data analysis, the raw spectra were deconvoluted using a 20000:40000 Da window and 1 Da resolution. The signal from masses 20000:30000 and 33000:40000 (which contained no peaks) was averaged and subtracted from the whole signal. The peaks of each species were integrated using a 100 Da window in every direction (reducing the window down to 10 Da did not change the results significantly).

### In-Gel Fluorescence Activity-Based Profiling

Mino cells were treated for 2 h with either 0.1% DMSO or the indicated concentrations of IA-alkyne, 2a, 2b, 2c. The cells were lysed with RIPA buffer (Sigma) and protein concentration was determined using BCA protein assay (Thermo Fisher Scientific). Lysates were then diluted to 2 mg/ml in PBS and clicked to TAMRA-azide. Click reaction was performed using a final concentration of 40 μM TAMRA-azide, 3 mM CuSO4, 3 mM Tris(3-hydroxypropyltriazolylmethyl)amine (THPTA, Sigma), and 3.7 mM Sodium L-ascorbate (Sigma) in a final volume of 60 μl. The samples were incubated at 25 degrees for 2 hours. 20 μl of 4×LDS sample buffer (NuPAGE, Thermo Fischer Scientific) was added followed by 10 min incubation at 70 degrees. The samples were then loaded on a 4-20% Bis-Tris gel (SurePAGE, GeneScript) and imaged using Typhoon FLA 9500 scanner.

### Buffer Stability Assay for Model Compounds

A sample of 100 μM of the electrophile (7c, 7d, 7k, 7f, 7e, 7m, 7n, 7o, 7q, 7r, 7s, 9c, 9a, and 9b) was incubated with 100 μM of 4-nitrocyano benzene as an internal standard in a 100 mM potassium phosphate buffer of pH 8.0. All solvents were bubbled with argon. Reaction mixtures were kept at 37° C. with shaking. After 4 days (unless otherwise mentioned), 5 μL from the reaction mixture were injected into the LC/MS to check the stability of the compounds.

### In-Gel Fluorescence Activity-Based Profiling

Mino cells were cultured in RPMI-medium supplemented with 15% FBS and 1% p/s, at 37° C. and 5% CO₂. The cells were treated for 2 h with either 0.1% DMSO or the indicated concentrations of 7d, 7f, 7n. For the competition experiment the cells were pre-incubated for 30 min with 1 μM Ibrutinib followed by 2 h incubation with 200 nM 7d, 200 nM 7f and 100 nM 7n. The cells were lysed with RIPA buffer (Sigma, R0278) and protein concentration was determined using BCA protein assay (Thermo Fisher Scientific, 23225). Lysates were then diluted to 2 mg/mL in PBS. Incubation with 7e was performed in lysates for 2 h at 25° C. Lysates with 7d and 7f were clicked to TAMRA-azide (Lumiprobe). For 7d “click” reaction was performed using a final concentration of 40 μM TAMRA-azide, 3 mM CuSO₄, 3 mM Tris(3-hydroxypropyltriazolylmethyl)amine (THPTA, Sigma), and 3.7 mM Sodium L-ascorbate (Sigma) in a final volume of 60 μL. For 7f the “click” reaction was performed by incubation with 40 μM TAMRA-azide. The samples were incubated at 25° C. for 2 h. 20 μL of 4×LDS sample buffer (NuPAGE, Thermo Fischer Scientific, NP0007) were added followed by 10 min incubation at 70° C. The samples were then loaded on a 4-20% Bis-Tris gel (SurePAGE, GeneScript) and imaged using Typhoon FLA 9500 scanner. 7d and 7f were scanned at 532 nm, 7n and 7e were scanned at 473 nm.

### BTK Activity in Cells

Mino cells were treated with either 0.1% DMSO or the indicated concentrations of Ibrutinib, IbrH, 7d and 7f for 1 h. The cells were then incubated with 10 μg/ml anti-human IgM (Jackson ImmunoResearch, 109-006-129) for 10 min at 37° C., harvested and immunoblots of phospho-BTK, total-BTK and b-actin were performed.

### B-Cell Response Experiment

Splenic cells from C57BL/6 mice were isolated by forcing spleen tissue through the mesh into PBS containing 2% fetal calf serum and 1 mM EDTA and red blood cells were depleted by lysis buffer. Cells were cultured in 96-well U-bottom dishes (1×106 cells/mL in RPMI 10% FCS) and incubated with Ibrutinib, 7d and 7f in different concentrations (1 nM, 10 nM, 100 nM, 1000 nM) for 24 h at 37° C. in 5% humidified CO2. Following a 24 h incubation, cells were stimulated with anti-IgM overnight (5 μg/mL, Sigma-Aldrich). Subsequently, cells were stained with anti-B220 (clone RA3-6B2, Biolegend) and anti-CD86 (clone GL-1, Biolegend) antibodies (anti-mouse CD86 biolegend 105008 1:400, anti-mouse/human CD45R/B220 biolegend 103212 1:400) for 30 min at 4° C. Single-cell suspensions were analyzed by a flow cytometer (CytoFlex, Beckman Coulter).

### Immunoblotting

Cell pellets were washed with ice-cold PBS and lysed using RIPA-buffer (Sigma, R0278). Lysates were clarified at 21,000 g for 15 min at 4° C. and protein concentration was determined using BCA protein assay (Thermo Fisher Scientific, 23225). Samples containing 50 μg total protein were prepared with 4×LDS sample buffer (NuPAGE, Thermo Fischer Scientific, NP0007) and were then resolved on a 4-20% bis-tris gel (GeneScript SurePAGE, M00657). Proteins were separated by electrophoresis and were then transferred to a nitrocellulose membrane (Bio-Rad, 1704158) using the Trans-Blot Turbo system (Bio-Rad). The membrane was blocked with 5% BSA in TBS-T (w/v) for 1 h at room temperature, washed ×3 times for 5 min with TBS-T and incubated with the following primary antibodies: rabbit anti phospho-BTK (#87141s, cell-signaling, 1:1000, over-night at 4° C.), mouse anti BTK (#56044s, cell-signaling, 1:1000, 1 h at room-temperature), mouse anti b-actin (#3700, cell-signaling, 1:1000, 1 h at room-temperature). Membrane was washed ×3 times for 5 min with TBS-T and incubated with the corresponding HRP-linked secondary antibody (Mouse #7076/Rabbit #7074, cell-signaling) for 1 h at room-temperature. EZ-ECL Kit (Biological Industries, 20-500-1000) was used to detect HRP-activity. The membrane was stripped using Restore stripping buffer (Thermo Fisher Scientific, 21059) after each secondary antibody before blotting with the next one.

### Half-Life Determination

Measurements with 7f were performed by pulse-labeling of BTK in Mino cells with 100 nM 7f for 1 h, followed by a wash with PBS×3 times to remove excess probe. The cells were incubated at 37° C. in a 5% humidified CO₂ incubator and harvested at the indicated time-points. Cell pellets were lysed with RIPA buffer, clicked with TAMRA-azide, proteins were separated by electrophoresis and imaged as described in detail in the In-gel fluorescence section. BTK's bands were quantified using ImageJ software and BTK levels at time-point zero were defined as 100%.

Measurements with cycloheximide (CHX) were performed by treating Mino cells with 20 g/ml CHX. Cells were harvested at the indicated time-points for subsequent analysis by immunoblotting of BTK and b-actin. Bands were quantified using ImageJ, BTK signal was normalized to b-actin, and levels at time-point zero were defined as 100%. For both methods, BTK levels vs. time-points were plotted and the data was fitted to One-phase decay in Prism 8 to calculate the half-life.

### In Vitro Activity Assays for BTK (Carried Out by Nanosyn, Santa Clara, CA, USA)

Test compounds were diluted in DMSO to a final concentration that ranged from 2 μM to 11.3 μM, while the final concentration of DMSO in all assays was kept at 1%. The compounds were incubated with BTK for 2 h in a 2× buffer containing the following: 1.2 nM BTK, 100 mM HEPES pH=7.5, 10 mM MgCl2, 2 mM DTT, 0.1% BSA, 0.01% Triton X-100, 20 μM sodium orthovanadate, and 20 μM beta-glycerophosphate. The reaction was initiated by 2-fold dilution into a solution containing 5 μM ATP and substrate. A reference compound staurosporine was tested similarly.

### B-Cell Response Experiment

Splenic cells from C57BL/6 mice were isolated by forcing spleen tissue through the mesh into PBS containing 2% fetal calf serum and 1 mM EDTA and red blood cells were depleted by lysis buffer. Cells were cultured in 96-well U-bottom dishes (1×10⁶ cells/mL in RPMI 10% FCS) and incubated with BTK inhibitors in different concentrations (1 nM, 10 nM, 100 nM, 1000 nM) for 24 hours at 370 in 5% humidified CO₂. Following a 24 hours incubation, cells were stimulated with anti-IgM overnight (5 μg/mL, Sigma-Aldrich). Subsequently, cells were stained with anti-B220 (clone RA3-6B2, Biolegend) and anti-CD86 (clone GL-1, Biolegend) antibodies for 30 minutes at 4° C. Single-cell suspensions were analyzed by a flow cytometer (CytoFlex, Beckman Coulter).

### Fluorescence Intensity Measurements for CoLDR Turn-on Probes

2 μM of BTK, EGFR, or K-RAS^{G12C} was added to 2 μM 3j, 4b, or 5a respectively. Control measurements were performed either without protein or compound and for BTK with pre-incubation with 2 μM non-covalent Ibrutinib for 30 minutes. Each condition was in triplicates in 20 mM Tris pH 8 50 mM NaCl for BTK and K-RAS^{G}12c, in 50 mM Tris pH 8.0, 100 mM NaCl for EGFR. fluorescent measurements were taken every 2 minutes for 2 hours for BTK and EGFR and every 10 minutes for 15 hours for K-RAS^{G}12c. At the end of the measurements, samples were injected directly into the LC/MS for labeling % quantification. K-Ras^{G12C} was expressed and purified as previously described⁶⁶, EGFR kinase domain was a generous gift from Prof. Michael Eck.

Fluorescence Intensity Measurements with if

The BTK kinase domain was diluted to 2 μM in the buffer, and 2 μM 7m was added by adding 1/100th volume from a 200 μM solution. Control measurements were performed without protein and BTK with preincubation with 4 μM Ibr-H/Ibrutinib for 5 min. Each condition was done in quadruplicate in 20 mM Tris pH 8.0 and 50 mM NaCl for BTK. Fluorescent measurements were taken every 2 min for 1 h for BTK/K-Ras^{G12C}. At the end of the measurements, samples were injected directly into the LC/MS for labeling quantification.

HTS with the Chemiluminescent Probe

High throughput screening was performed with the Selleck compound collection at 10 μM in 1536-well white plates (Nanc, cat 264712), using GNF WDII washer/dispenser (Novartis, USA). BTK was preincubated with compounds for 15 minutes followed by the addition of a 3k luminescence probe. The screen was performed with 0.75 μM BTK and 1.5 μM of probe in 20 mM Tris pH 8 50 mM NaCl 0.1% BSA 1 mM DTT final concentration. Luminescence was recorded after 30 minutes using a BMG PheraStar plate reader.

### Example 1

### Synthesis of Compounds of this Invention

### Synthesis of (R)-7-((2-(3-(4-amino-3-(4-phenoxyphenyl)-1H-pyrazolo[3,4-d]pyrimidin-1-yl)piperidine-1-carbonyl)allyl)oxy)-2H-chromen-2-one (3j)

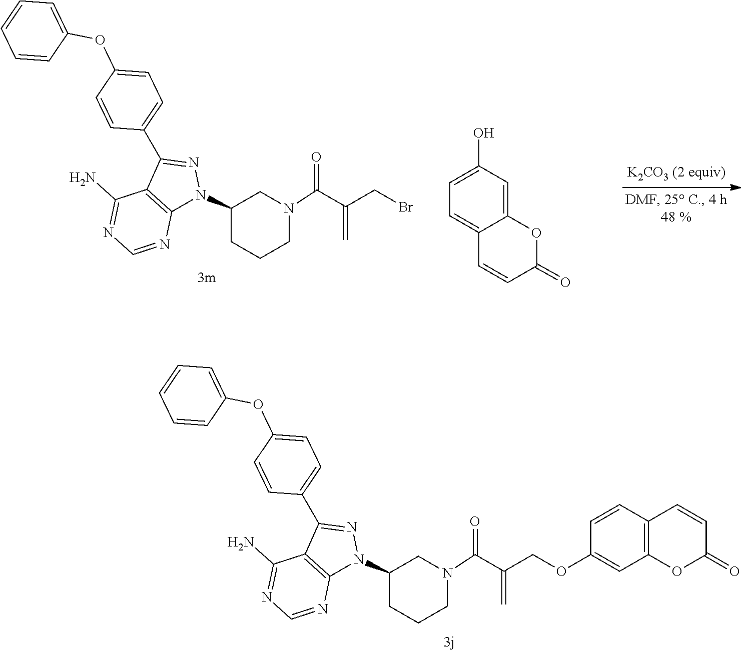 [SMILES: C=C(CBr)C(=O)N1CCC[C@@H](n2nc(-c3ccc(Oc4ccccc4)cc3)c3c(N)ncnc32)C1 ; O=c1ccc2ccc(O)cc2o1 ; C=C(COc1ccc2ccc(=O)oc2c1)C(=O)N1CCC[C@@H](n2nc(-c3ccc(Oc4ccccc4)cc3)c3c(N)ncnc32)C1]

To a stirred solution of bromo compound (3m) (26.5 mg, 0.05 mmol) in dry DMF (1 mL), 7-hydroxy coumarin (8.9 mg, 0.055 mmol) and K₂CO₃ (15.2 mg, 0.11 mmol), were added at 25° C. under an N₂ atmosphere. The reaction mixture was allowed to stir at room temperature for 4 h. After completion (as monitored by LC-MS), the reaction mixture was quenched with H₂O (2 mL). The aqueous layer was extracted with EtOAc (3×3 mL). The organic layer was washed with brine and concentrated in vacuo. The crude product was purified by High-Pressure Liquid Chromatography (HPLC) using water:ACN (0.1% formic acid) solvent gradient to afford white solid (48%, 14.8% yield).

¹H NMR (500 MHz, CD₃OD) δ 1.73 (m, 1H), 2.13 (br. s., 1H), 2.30 (br. s., 1H), 2.38 (br. s., 1H), 3.60 (br. s., 1H), 3.88 (dd, J=12.8, 9.2 Hz, 1H), 3.93-4.07 (m, 1H), 4.26-4.49 (m, 2H), 4.79-4.85 (m, 1H), 5.02 (s, 2H), 5.49 (s, 1H), 5.71 (br. s., 1H), 6.19-6.33 (m, 1H), 7.02 (br. s., 2H), 7.11 (br. s., 3H), 7.15-7.23 (m, 2H), 7.42 (t, J=7.6 Hz, 2H), 7.54 (br. s., 1H), 7.68 (d, J=8.7 Hz, 2H), 7.79-7.93 (m, 1H), 8.43 (s, 1H); ¹³C NMR (125 MHz, CD₃OD) δ 23.1, 29.0, 45.2, 50.6, 54.0, 69.0, 96.8, 101.4, 112.4, 112.7, 113.1, 118.7, 119.3, 125.7, 129.2, 129.7, 129.9, 139.3, 144.2, 146.5, 147.0, 151.8, 152.9, 155.5, 156.3, 159.2, 161.5, 169.5.

### Methyl (E)-3-(5-(((1r,3r,5R,7S)-adamantan-2-ylidene)(methoxy)methyl)-2-((2-((R)-3-(4-amino-3-(4-phenoxyphenyl)-1H-pyrazolo[3,4-d]pyrimidin-1-yl)piperidine-1-carbonyl)allyl)oxy)-3-chlorophenyl)acrylate (3l)

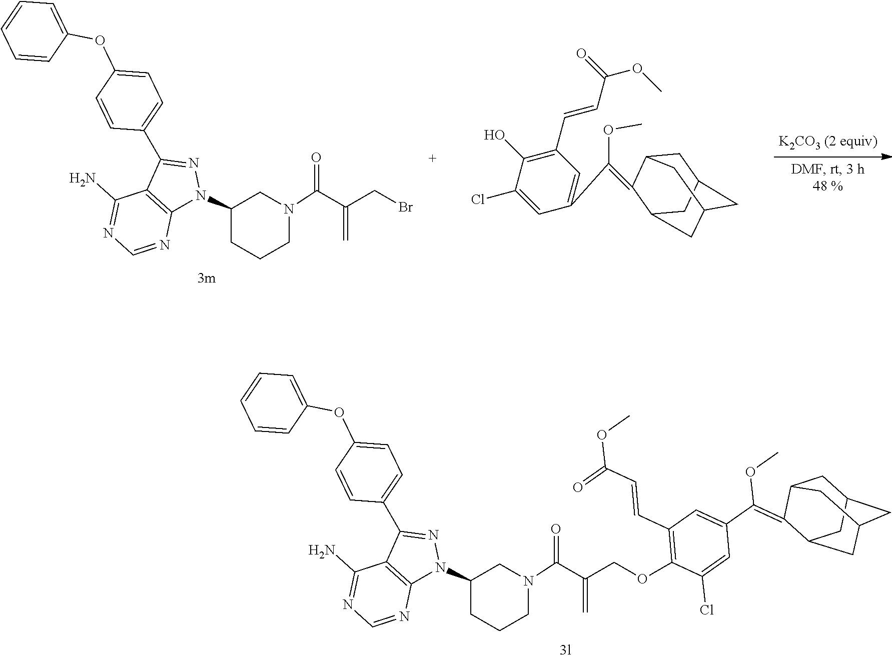 [SMILES: C=C(CBr)C(=O)N1CCC[C@@H](n2nc(-c3ccc(Oc4ccccc4)cc3)c3c(N)ncnc32)C1 ; COC(=O)/C=C/c1cc(C(OC)=C2C3CC4CC(C3)CC2C4)cc(Cl)c1O ; C=C(COc1c(Cl)cc(C(OC)=C2C3CC4CC(C3)CC2C4)cc1/C=C/C(=O)OC)C(=O)N1CCC[C@@H](n2nc(-c3ccc(Oc4ccccc4)cc3)c3c(N)ncnc32)C1]

To a stirred solution of bromo compound (3m) (26.5 mg, 0.05 mmol) in dry DMF (1 mL), phenol (4.83 uL, 0.055 mmol) and K₂CO₃ (15.2 mg, 0.11 mmol), were added at 25° C. under an N₂ atmosphere. The reaction mixture was allowed to stir at room temperature for 4 h. After completion (as monitored by LC-MS), the reaction mixture was quenched with H₂O (2 mL). The aqueous layer was extracted with EtOAc (3×3 mL). The organic layer was washed with brine and concentrated in vacuo. The crude product was purified by High-Pressure Liquid Chromatography (HPLC) using water:ACN (0.1% formic acid) solvent gradient to afford white solid (23.1 mg, 55% yield).

¹H NMR (400 MHz, CD₃OD) δ 1.83 (br. s., 2H), 1.86-2.00 (m, 7H), 2.04 (br. s., 4H), 2.15 (br. s., 2H), 2.35 (br. s., 1H), 2.48 (q, J=9.7 Hz, 1H), 3.34 (br. s., 3H), 3.77-3.85 (br. s., 3H), 3.97-4.19 (br. s., 2H), 4.41-4.61 (m, 2H), 4.77 (br. s., 1H), 4.86 (br. s., 1H), 5.10-5.21 (m, 1H), 5.57 (br. s., 1H), 5.79-5.87 (br. s., 1H), 6.58 (d, J=16.1 Hz, 1H), 7.11-7.34 (m, 6H), 7.48-7.57 (m, 2H), 7.72 (br. s., 3H), 7.82 (d, J=16.3 Hz, 1H), 7.99 (d, J=15.2 Hz, 1H), 8.21 (br. s., 1H); ¹³C NMR (125 MHz, CD₃OD) δ 29.8, 29.9, 31.3, 34.6, 38.2, 39.7, 39.8, 40.2, 40.3, 43.1, 46.8, 52.4, 54.3, 55.2, 57.7, 75.6, 75.8, 98.2, 120.1, 120.9, 125.4, 126.9, 129.6, 130.7, 131.3, 131.4, 139.6, 141.1, 141.4, 148.4, 153.4, 154.7, 155.0, 157.8, 160.7, 168.6, 170.9.

### Methyl (E)-3-(2-((2-((R)-3-(4-amino-3-(4-phenoxyphenyl)-1H-pyrazolo[3,4-d]pyrimidin-1-yl)piperidine-1-carbonyl)allyl)oxy)-3-chloro-5-((1r,3r,5R,7R)-4′-methoxyspiro[adamantane-2,3′-[1,2]dioxetan]-4′-yl)phenyl)acrylate (3k)

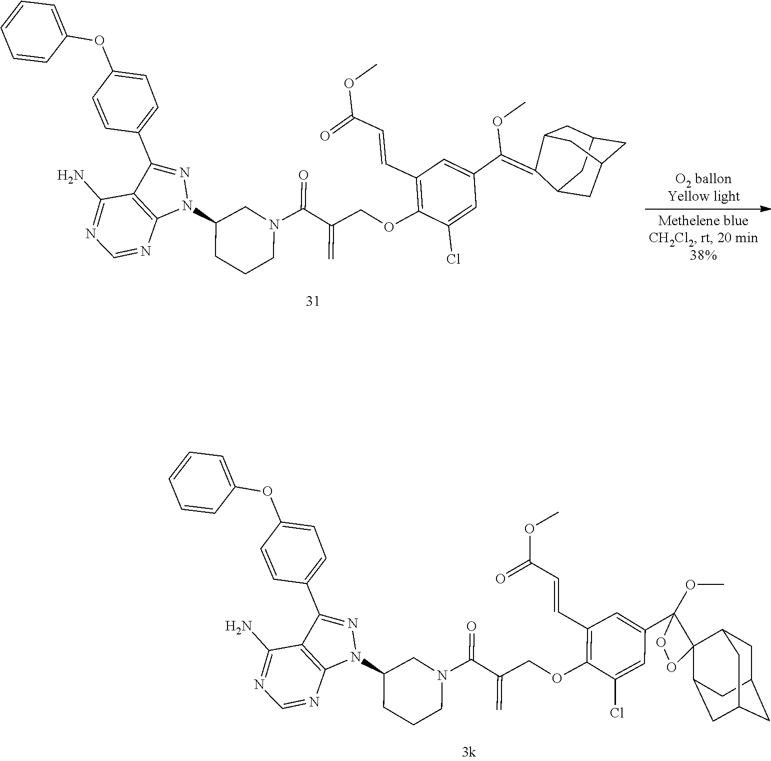 [SMILES: C=C(COc1c(Cl)cc(C(OC)=C2C3CC4CC(C3)CC2C4)cc1/C=C/C(=O)OC)C(=O)N1CCC[C@@H](n2nc(-c3ccc(Oc4ccccc4)cc3)c3c(N)ncnc32)C1 ; C=C(COc1c(Cl)cc(C2(OC)OOC23C2CC4CC(C2)CC3C4)cc1/C=C/C(=O)OC)C(=O)N1CCC[C@@H](n2nc(-c3ccc(Oc4ccccc4)cc3)c3c(N)ncnc32)C1]

To a stirred solution of enol ether (3l) 8.4 mg, 0.01 mmol) in dry DCM (1 mL), methylene blue was added at 25° C. and in the presence of yellow light. The reaction mixture was bubbled with oxygen and allowed to stir for 20 min. After completion (as monitored by LC-MS), the DCM was concentrated in vacuo. The crude product was purified by High-Pressure Liquid Chromatography (HPLC) using water:ACN (0.1% formic acid) solvent gradient to afford white solid (4.36 mg, 38% yield).

¹H NMR (500 MHz, CD₃OD) δ 1.55-1.74 (m, 6H), 1.75-1.87 (m, 5H), 1.96 (s, 2H), 2.05 (br. s., 1H), 2.13-2.30 (m, 2H), 2.38 (br. s., 2H), 3.20 (s, 3H), 3.67 (br. s., 3H), 3.79 (br. s., 1H), 3.92 (br. s., 1H), 4.27-4.50 (m, 2H), 4.65 (br. s., 1H), 5.06 (br. s., 2H), 5.50 (br. s., 1H), 5.72 (br. s., 1H), 5.79 (br. s., 1H), 6.49-6.63 (m, 1H), 7.13 (br. s., 5H), 7.18-7.25 (m, 1H), 7.44 (t, J=7.2 Hz, 2H), 7.64 (t, J=8.5 Hz, 2H), 7.80 (br. s., 1H), 7.93 (d, J=8.3 Hz, 1H): ¹³C NMR (125 MHz, CD₃OD) δ 27.6, 28.0, 28.9, 33.0, 33.4, 34.6, 35.1, 37.9, 38.7, 49.9, 50.3, 52.7, 52.7, 53.5, 75.8, 97.5, 113.1, 119.1, 119.6, 120.3, 121.0, 125.6, 127.4, 127.6, 131.4, 131.5, 131.6, 134.9, 137.0, 138.8, 141.4, 150.0, 158.0, 160.8, 162.4, 168.5, 171.3.

### 2-(((2-oxo-2H-chromen-7-yl)oxy)methyl)acrylic acid

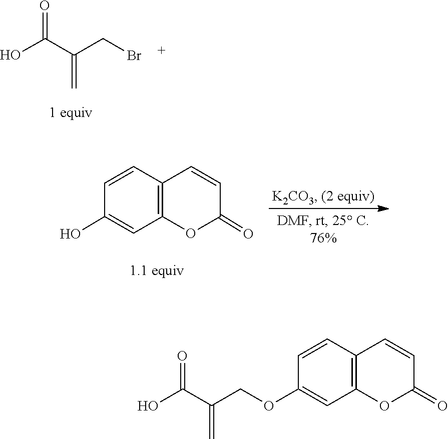

To a stirred solution of 7-hydroxy coumarin (92 mg, 0.56 mmol) in DMF (3 ML) was added NaH (44.8 mg, 1.12 mmol) and bromo methacrylic acid (90.1 mg, 0.56 mmol) and the reaction mixture was allowed to stir at 25° C. for 2 h under nitrogen atmosphere. After completion of the reaction, monitored by TLC, the reaction mixture was quenched with water and extracted with EtOAc (3×10 mL). The combined organic layer was washed with brine solution (3×15) and the organic layer was dried in Na₂SO₄ and then evaporated under reduced pressure to give the crude acid which was purified using silica gel chromatography using hexane: ethyl acetate mixture to obtain white solid (105 mg, 76%)

¹H NMR (500 MHz, CDCl₃) δ 4.82 (s, 2H), 6.03 (s, 1H), 6.26 (d, J=9.4 Hz, 1H), 6.50 (s, 1H), 6.86 (s, 1H), 6.89 (d, J=10.7 Hz, 1H), 7.38 (d, J=8.5 Hz, 1H), 7.64 (d, J=9.5 Hz, 1H), 8.04 (s, 2H): ¹³C NMR (125 MHz, CDCl₃) δ 66.5, 102.0, 112.9, 113.3, 127.9, 128.9, 135.1, 143.5, 155.8, 161.3, 161.5, 168.1.

### N-(4-((3-chloro-4-fluorophenyl)amino)-7-methoxyquinazolin-6-yl)-2-(((2-oxo-2H-chromen-7-yl)oxy)methyl)acrylamide (4b)

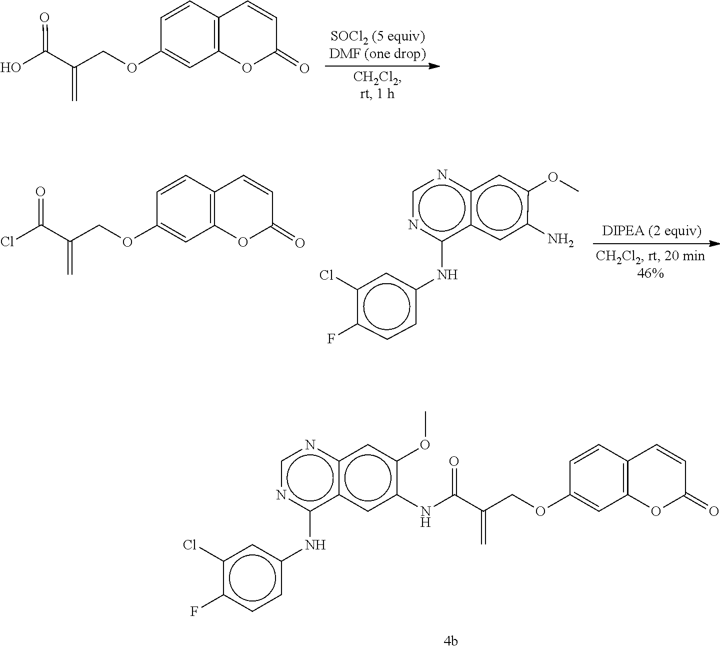

To a stirred solution of 2-(((2-oxo-2H-chromen-7-yl)oxy)methyl)acrylic acid (12.3 mg, 0.05 mmol) in CH₂Cl₂ (1 mL) were added SOCl₂ (18.1 uL, 0.25 mmol) and DMF (3.9 uL, 0.05 mmol) and the reaction mixture was allowed to stir at 25° C. for 4 h. After completion (as monitored by LC-MS), the reaction mixture was concentrated in vacuo. The crude acid chloride was dissolved in CH₂Cl₂ and slowly to the solution of afatinib amine (0.05 mmol, 15.9 mg) and DIPEA (17.8 uL, 0.1 mmol) was treated with purified by High-Pressure Liquid Chromatography (HPLC) using water:ACN (0.1% formic acid) solvent gradient to afford white solid (12.5 mg, 46% yield).

¹H NMR (500 MHz, DMSO-d₆) δ 3.97 (br. s., 3H), 5.03 (br. s., 2H), 6.03 (br. s., 1H), 6.27-6.39 (m, 2H), 7.06 (d, J=6.5 Hz, 1H), 7.12 (br. s., 1H), 7.32 (br. s., 1H), 7.37-7.46 (m, 1H), 7.69 (d, J=8.3 Hz, 1H), 7.80 (br. s., 1H), 8.02 (d, J=9.1 Hz, 1H), 8.56 (br. s., 1H), 8.82 (br. s., 1H), 9.59 (br. s., 1H), 9.85 (br. s., 1H); ¹³C NMR (125 MHz, DMSO-d₆) δ 56.4, 67.6, 101.7, 106.9, 108.8, 112.8, 112.8, 112.8, 113.0, 116.4, 116.5, 117.1, 122.4, 123.5, 124.9, 126.8, 129.6, 136.8, 138.6, 144.3, 149.5, 154.1, 155.3, 155.9, 156.8, 160.2, 160.9, 164.4.

### 6-fluoro-7-(2-fluoro-6-hydroxyphenyl)-1-(2-isopropyl-4-methylpyridin-3-yl)-4-((S)-2-methyl-4-(2-(((2-oxo-2H-chromen-7-yl)oxy)methyl)acryloyl)piperazin-1-yl)pyrido[2,3-d]pyrimidin-2(1H)-one (5a)

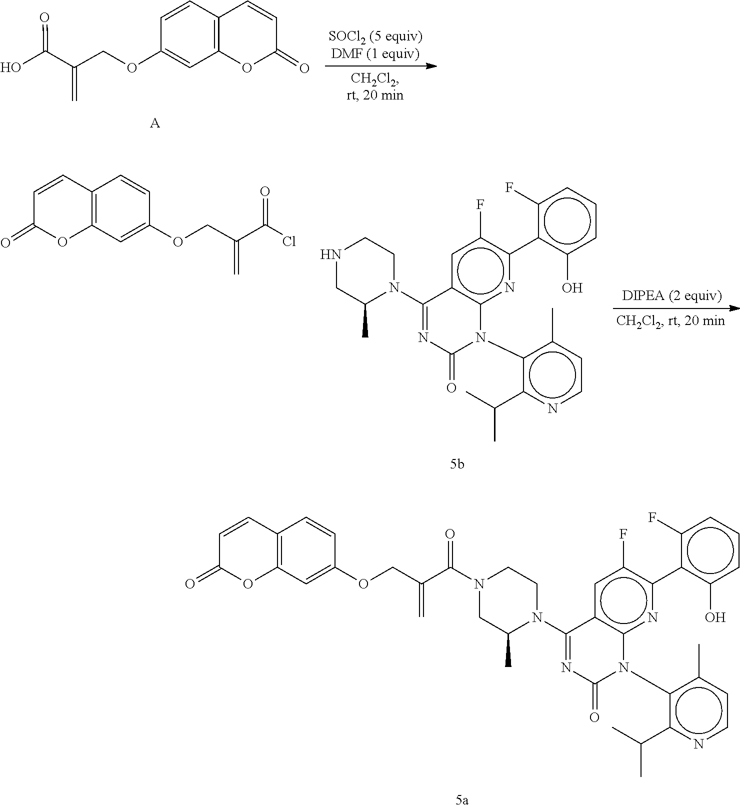

To a stirred solution of 2-(((2-oxo-2H-chromen-7-yl)oxy)methyl)acrylic acid (A) (1.23 mg, 0.05 mmol) in CH₂Cl₂ (200 uL) were added SOCl₂ (1.81 uL, 0.25 mmol) and DMF (0.4 uL, 0.05 mmol) and the reaction mixture was allowed to stir at 25° C. for 4 h. After completion (as monitored by LC-MS), the reaction mixture was concentrated in vacuo. The crude acid chloride was dissolved in CH₂Cl₂ and slowly added to the solution of amine 5b (0.005 mmol, 2.53 mg) and DIPEA (1.78 uL, 0.01 mmol) and allowed to stir at 25° C. under N₂ atmosphere. After completion (as monitored by LC-MS), the reaction mixture was concentrated in vacuo and purified by High-Pressure Liquid Chromatography (HPLC) using water:ACN (0.1% formic acid) solvent gradient to afford white solid 5a in (2.8 mg, 42% yield).

¹H NMR (500 MHz, CD₃OD) δ 1.15 (t, J=7.1 Hz, 4H), 1.24-1.35 (m, 6H), 1.37-1.44 (m, 4H), 2.22 (d, J=7.7 Hz, 3H), 3.02-3.11 (m, 3H), 4.47 (d, J=19.8 Hz, 1H), 4.53 (br. s., 1H), 4.96 (br. s., 2H), 5.52-5.59 (m, 1H), 5.81 (s, 1H), 6.29 (d, J=9.5 Hz, 1H), 6.63 (t, J=8.9 Hz, 1H), 6.68 (s, 1H), 7.04 (s, 1H), 7.18 (d, J=8.5 Hz, 1H), 7.26 (d, J=7.3 Hz, 2H), 7.41-7.48 (m, 1H), 7.59 (d, J=8.5 Hz, 1H), 7.64-7.73 (m, 2H), 7.91 (d, J=9.5 Hz, 1H), 8.40 (d, J=8.9 Hz, 1H), 8.54-8.60 (m, 1H).

### Synthesis of 6a:

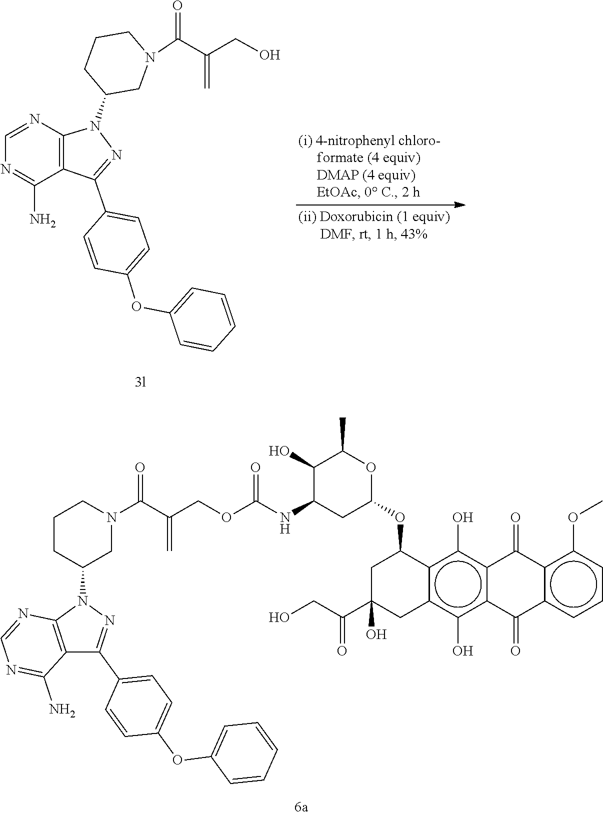

To a stirred solution of alcohol (3l) (23.5 mg, 0.05 mmol) in ethyl acetate (2 mL) were added 4-nitrophenyl chloroformate (40.8 mg, 0.2 mmol) and 4-Dimethylaminopyridine (24.4 mg, 0.2 mmol) at 0° C. The reaction mixture was allowed to stir at room temperature for 2 h. After completion of the reaction (as monitored by LC-MS, the reaction mixture is quenched with 0.1 N HCl (2 mL) and the aqueous layer was extracted with EtOAc (3×4 mL). The organic layer was concentrated in vacuo. The crude reaction mixture was dissolved in DMF (0.5 mL) and added to doxorubicin (27.2 mg, 0.05 mmol). The reaction is further allowed to stir for 1 hour at room temperature. After completion of the reaction, methanol was concentrated and the crude product was purified by High Pressure Liquid Chromatography (HPLC) using water:ACN (0.1% formic acid) solvent gradient to afford white solid 6a in (22.4 mg, 43% yield).

### Synthesis of 6b:

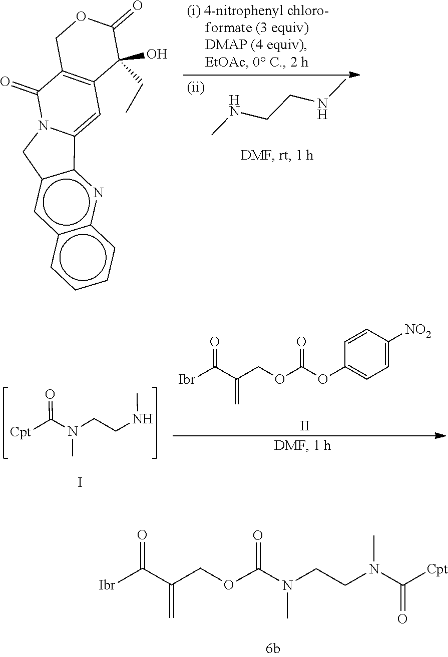

Camptothecin (15 mg, 0.05 mmol) and PNP-chloroformate (40.8 mg, 0.2 mmol) were dissolved in methylene-chloride (2 mL) at 0° C., followed by the addition of DMAP (24.4 mg, 0.2 mmol). The resulting clear solution was stirred at room temperature for 1 h. The reaction was monitored by lc-ms. After completion, the mixture was diluted with 2 mL of methylene-chloride and washed with 3 mL HCl 0.1 N. The organic layer was dried over magnesium sulfate, concentrated under reduced pressure to 10 mL, and precipitated with ether. The precipitated solid was filtered and dried to give crude compound. The crude p-nitrophenyl camptothecin carbonate was dissolved in DMF, and N,N′-dimethyl ethylene-diamine (3 mg, 0.05 mmol) was added. The mixture was stirred for 30 min, and the DMF was removed under reduced pressure to give the compound I in crude form.

In another vial, to a stirred solution of alcohol (3l) (23.5 mg, 0.05 mmol) in ethyl acetate (0.5 mL) were added 4-nitrophenyl chloroformate (40.8 mg, 0.2 mmol) and 4-Dimethylaminopyridine (24.4 mg, 0.2 mmol) at 0° C. The reaction mixture was allowed to stir at room temperature for 2 h. After completion of the reaction (as monitored by LC-MS, the reaction mixture is quenched with 0.1 N HCl (2 mL) and the aqueous layer was extracted with EtOAc (3×3 mL). The organic layer was concentrated in vacuo to give the crude product II.

To this crude product of I in DMF p-nitrophenyl Ibrutinib carbonate II was added and allowed to stir for 1 h. After completion of the reaction (as monitored by LC-MS), and the aqueous layer was extracted with EtOAc (3×3 mL). The organic layer was concentrated in vacuo to give the crude product was purified by High Pressure Liquid Chromatography (HPLC) using water:ACN (0.1% formic acid) solvent gradient to afford white solid 6b in (18.4 mg, 39% yield).

### Synthesis of 6c:

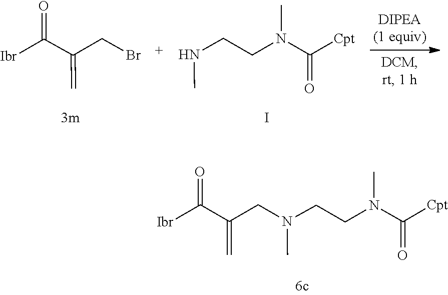

To a stirred solution of bromo compound (3m) (26.5 mg, 0.05 mmol) in anhydrous DCM (1 mL) were added crude camptothecine amine (I) (0.05 mmol, synthesis of I was given above) and DIPEA (9.8 uL, 0.055 mmol) at 25° C. The reaction mixture was allowed to stir at room temperature for one hour. After completion of the reaction (as monitored by LC-MS), CH₂Cl₂ was concentrated in vacuo. The crude product was purified by High-Pressure Liquid Chromatography (HPLC) using water: ACN (0.1% formic acid) solvent gradient to afford white solid (24 mg, 45% yield) of compound 6c.

### Synthesis of 6d:

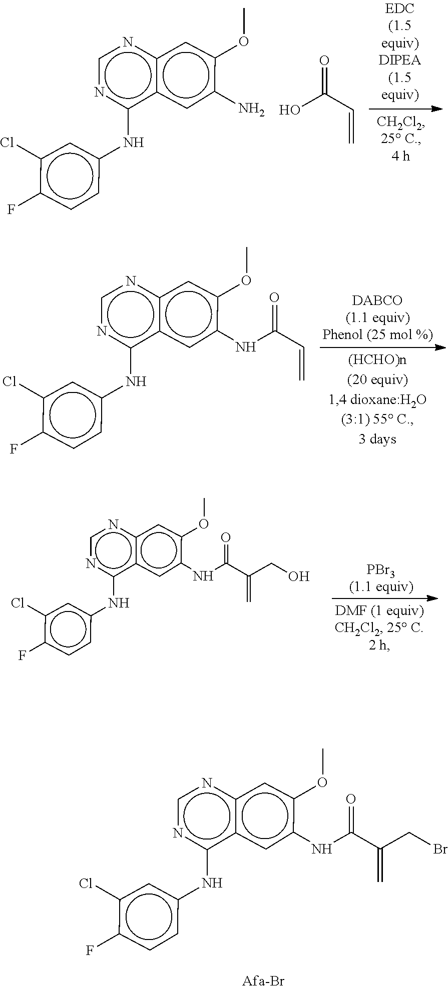

Afa-Br compound was prepared using the same procedures shown in the synthesis of 3m where afatinib-amine was used instead of Ibr-H

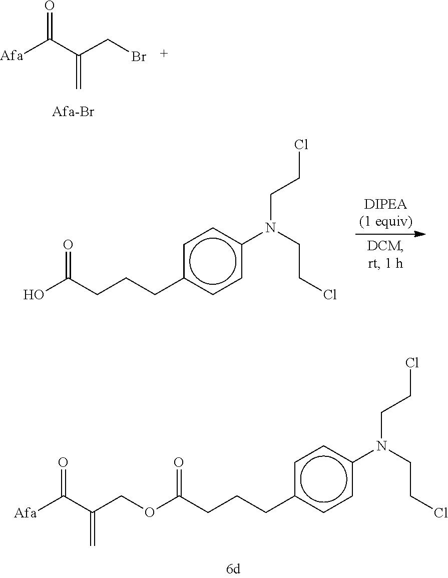

To a stirred solution of Afa-Br compound (23.5 mg, 0.05 mmol) in anhydrous DCM (1 mL) were added chlorambucil (15.0 mg, 0.05 mmol) and DIPEA (9.8 uL, 0.055 mmol) at 25° C. The reaction mixture was allowed to stir at room temperature for one hour. After completion of the reaction (as monitored by LC-MS), CH₂Cl₂ was concentrated in vacuo. The crude product was purified by High-Pressure Liquid Chromatography (HPLC) using water: ACN (0.1% formic acid) solvent gradient to afford white solid of compound 6d (11 mg, yield-32%).

### Synthesis of 6e

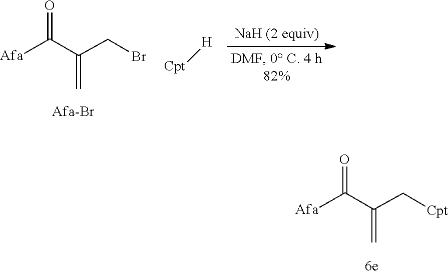

To a stirred solution of camptothecin (15 mg, 0.05 mmol) in anhydrous DMF (0.5 mL) were added NaH (4 mg (60% in mineral oil), 0.1 mmol)) at 0° C. After stirring for 5 min, Afa-Br (23 mg, 0.05 mmol) was added at 0° C. under N₂ atm. The reaction mixture was stirred at room temperature for 4 h under N₂ atm and quenched with H₂O (2 mL) at 0° C. The aqueous layer was extracted with EtOAc (3×3 mL). The organic layer was washed with brine and concentrated in vacuo. The crude product was purified by High Pressure Liquid Chromatography (HPLC) using water:ACN (0.1% formic acid) solvent gradient to afford white solid 6e in (8.4 mg, 22% yield).

### Synthesis of 6f:

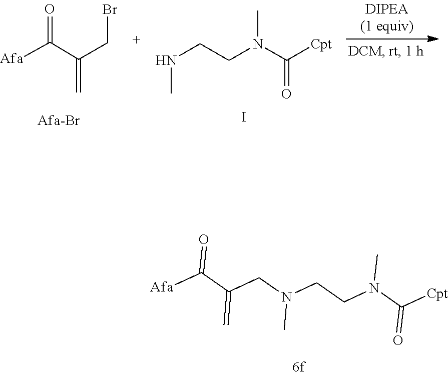

6f compound was prepared using the same scheme and procedures shown for the synthesis of 6c whereas 3m was replaced with Afa-Br

### Synthesis of 6g:

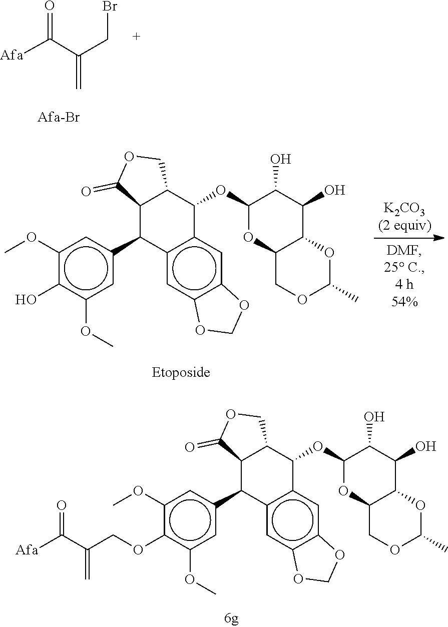

To a stirred solution of etoposide (29.5 mg, 0.05 mmol) in anhydrous DMF (0.8 mL) was added K₂CO₃ (27.6 mg, 0.1 mmol) at 25° C. After stirring for 5 min, Afa-Br (23 mg, 0.05 mmol) was added at 25° C. under N₂ atm. The reaction mixture was stirred at room temperature for 4 h under N₂ atm and quenched with H₂O (2 mL). The aqueous layer was extracted with EtOAc (3×3 mL). The organic layer was washed with brine and concentrated in vacuo. The crude product was purified by High Pressure Liquid Chromatography (HPLC) using water:ACN (0.1% formic acid) solvent gradient to afford white solid 6g in 28.1 mg (58% yield).

### Synthesis of 6h

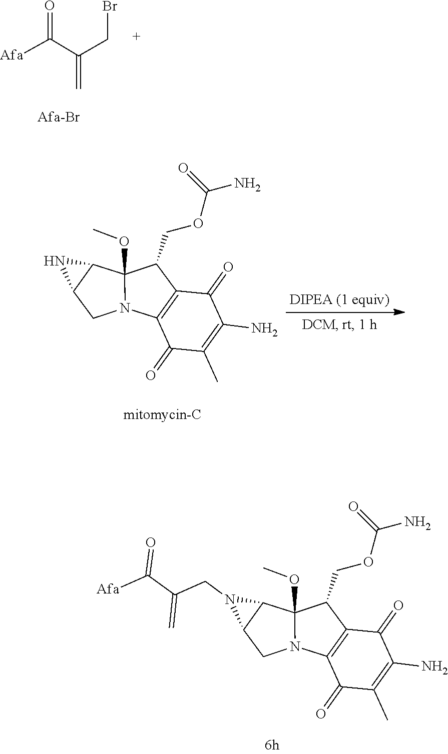

To a stirred solution of Afa-Br compound (23.5 mg, 0.05 mmol) in anhydrous DCM (1 mL) were added mitomycin-C (15.0 mg, 0.05 mmol) and DIPEA (9.8 uL, 0.055 mmol) at 25° C. The reaction mixture was allowed to stir at room temperature for one hour. After completion of the reaction (as monitored by LC-MS), CH2Cl2 was concentrated in vacuo. The crude product was purified by High-Pressure Liquid Chromatography (HPLC) using water: ACN (0.1% formic acid) solvent gradient to afford white solid of compound 6h (4 mg, 11% yield).

### Synthesis of 6i:

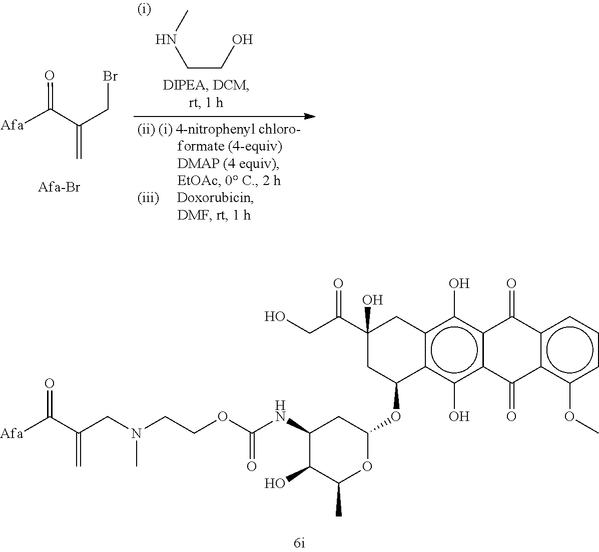

To a stirred solution of Afa-Br compound (23.5 mg, 0.05 mmol) in anhydrous DCM (1 mL) were added N-methyl ethanol amine (3.7 mg, 0.05 mmol) and DIPEA (9.8 uL, 0.055 mmol) at 25° C. The reaction mixture was allowed to stir at room temperature for one hour. After completion of the reaction (as monitored by LC-MS), CH₂Cl₂ was washed with water and extracted with CH₂Cl₂ (3×2 mL) and the organic layer was concentrated in vacuo. The alcohol and PNP-chloroformate (40.8 mg, 0.2 mmol) were dissolved in methylene-chloride (2 mL) at 0° C., followed by the addition of DMAP (24.4 mg, 0.2 mmol). The resulting clear solution was stirred at room temperature for 1 h. The reaction was monitored by lc-ms. After completion, the mixture was diluted with 2 mL of methylene-chloride and washed with 3 mL HCl 0.1 N. The organic layer was dried over magnesium sulfate, concentrated under reduced pressure to 10 mL, and precipitated with ether. The precipitated solid was filtered and dried to give crude compound. The crude carbonate was dissolved in DMF, and doxorubicin (27 mg, 0.05 mmol) was added. The mixture was stirred for 30 min, and the DMF was removed under reduced pressure to give the compound 6i (5 mg, 10% yield).

### Synthesis of 7a:

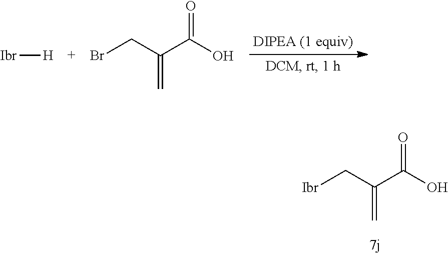

To a stirred solution of Ibr-H (387 mg, 1 mmol) in anhydrous DCM (6 mL), DIPEA (178 uL, 1 mmol) and 2-(bromomethyl)acrylic acid (161 mg, 1 mmol) were added at 25° C. The reaction mixture was stirred at room temperature for 1 h. After completion of the reaction (as monitored by LC-MS), CH₂Cl₂ was concentrated in vacuo to obtain the crude carboxylic acid.

The crude carboxylic acid was purified using flash column chromatography in hexane: ethyl acetate:methanol solvent system to get pure carboxylic acid 7j (334 mg, yield 72%)

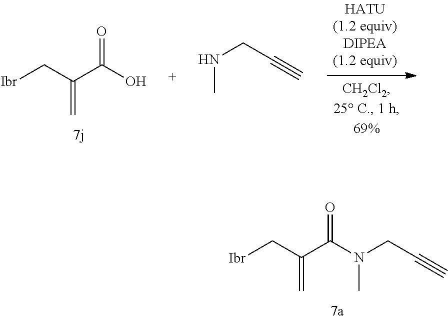

To a solution of carboxylic acid (23.5 mg, 0.05 mmol) in CH₂Cl₂ (1 mL), HATU (23 mg, 0.06 mmol), DIPEA (11 uL, 0.6 mmol) and N-methylprop-2-yn-1-amine hydrochloride (6.3 mg, 0.6 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 7a in 18 mg (69% yield).

### Synthesis of 7b:

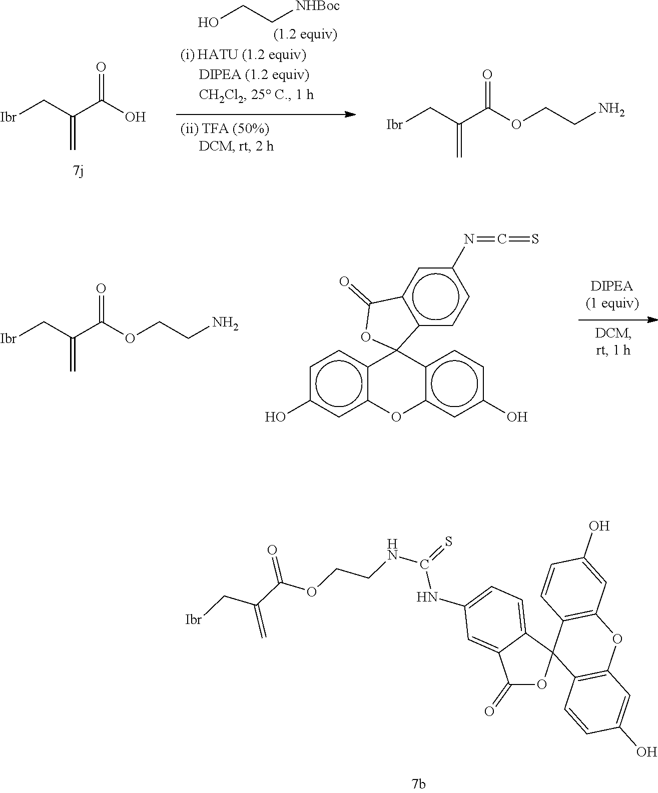

Synthesis of 7b is same as 7e where N-boc ethylene diamine was replaced with N-boc ethanol amine.

### Synthesis of 7c:

2 uL of 100 mM solution of Ibr-H and 2 uL of 100 mM solution of ethyl bromo methacrylate were mixed and vertexed for every 5 minutes for 30 minutes. The resulting 4 uL of 50 mM solution was used as such for the invitro binding with BTK.

### Synthesis of 7d:

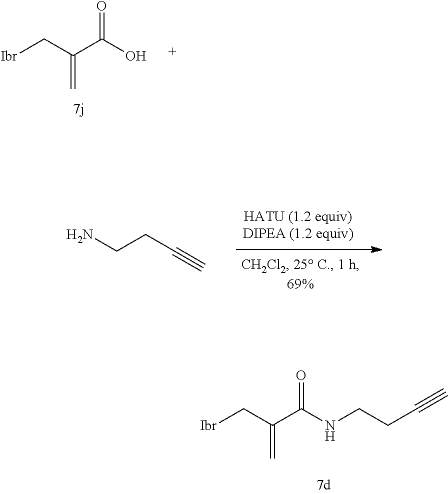

To a solution of carboxylic acid (23.5 mg, 0.05 mmol) in CH₂Cl₂ (1 mL), HATU (23 mg, 0.06 mmol), DIPEA (11 uL, 0.6 mmol) and but-3-yn-1-amine hydrochloride (6.3 mg, 0.6 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 7a in 18 mg (69% yield).

### Synthesis of 7e:

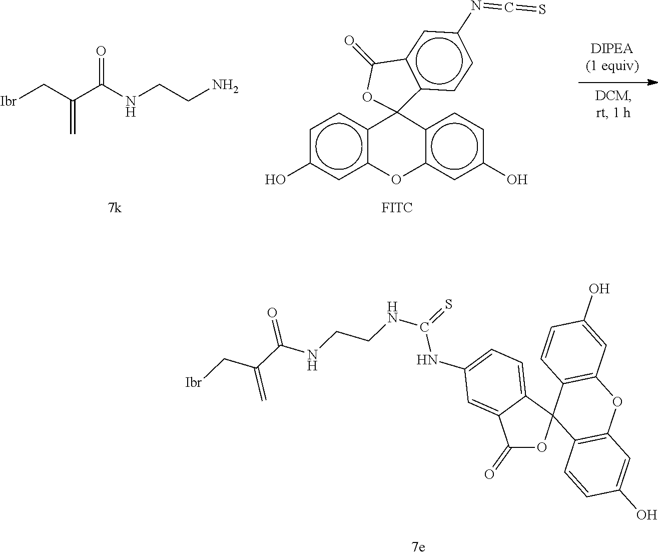

To a stirred solution of amine-7m (0.05 mmol, 26 mg) in anhydrous DCM (1 mL), DIPEA (9 uL, 0.05 mmol) and FITC (19 mg, 0.05 mmol) were added at 25° C. The reaction mixture was stirred at room temperature for 1 h. After completion of the reaction (as monitored by LC-MS), CH₂Cl₂ was concentrated in vacuo to obtain the crude carboxylic acid. the crude product was purified by High Pressure Liquid Chromatography (HPLC) using water:ACN (0.1% formic acid) solvent gradient to afford white solid 7e in 29 mg (66.0% yield).

### Synthesis of 7f:

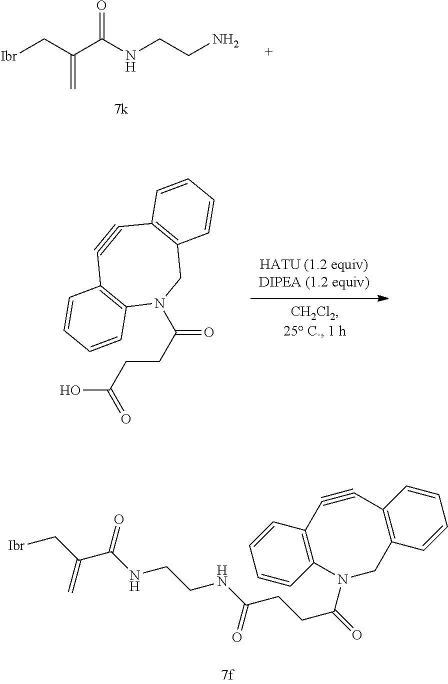

To a solution of carboxylic acid (15.3 mg, 0.05 mmol) in CH₂Cl₂ (1 mL), HATU (23 mg, 0.12 mmol), DIPEA (11 uL, 0.12 mmol) and amine 7m (26 mg, 0.12 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 7f in 15 mg (37.5% yield).

### Synthesis of 7g:

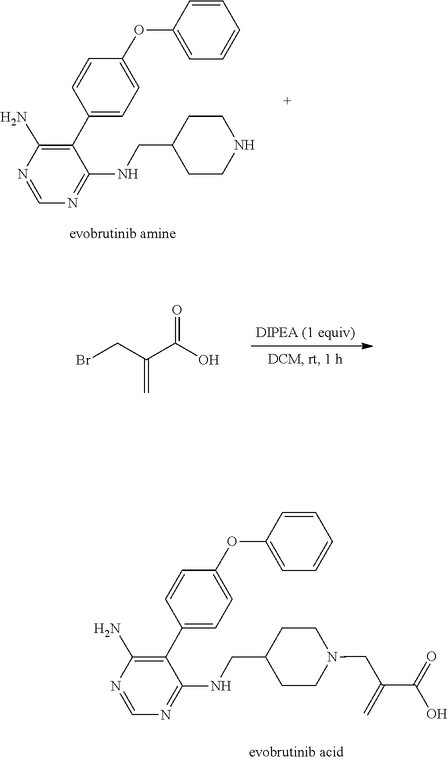

To a stirred solution of evobrutinib amine (37.5 mg, 0.1 mmol) in anhydrous DCM (1 mL), DIPEA (17.8 uL, 0.1 mmol) and 2-(bromomethyl)acrylic acid (16.1 mg, 0.1 mmol) were added at 25° C. The reaction mixture was stirred at room temperature for 1 h. After completion of the reaction (as monitored by LC-MS), CH₂Cl₂ was concentrated in vacuo to obtain the crude carboxylic acid. the crude product was purified by High Pressure Liquid Chromatography (HPLC) using water:ACN (0.1% formic acid) solvent gradient to afford white solid of evobrutinib acid in 40 mg (78% yield).

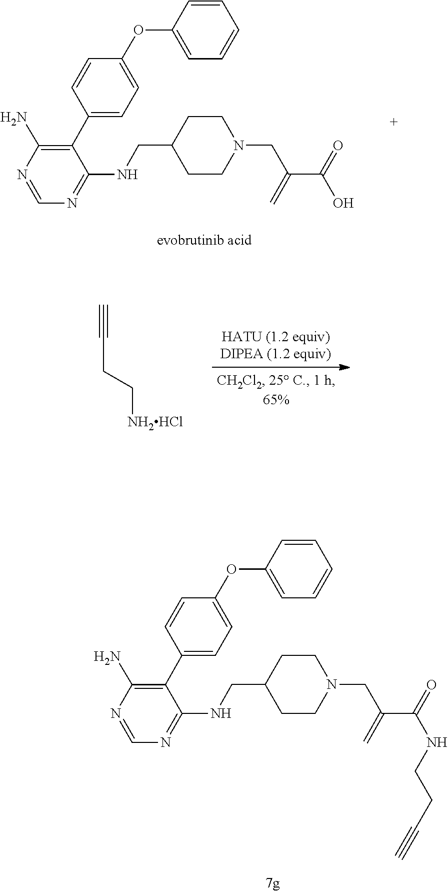

To a solution of evobrutinib acid (23 mg, 0.05 mmol) in CH₂Cl₂ (1 mL), HATU (23 mg, 0.06 mmol), DIPEA (11 uL, 0.06 mmol) and but-3-yn-1-aminehydrochloride (6.3 mg, 0.6 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 7a in 14 mg (56% yield).

### Synthesis of 7h:

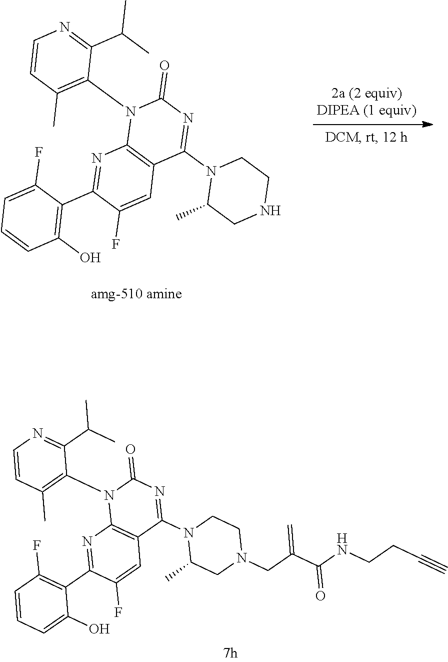 [SMILES: Cc1ccnc(C(C)C)c1-n1c(=O)nc(N2CCNC[C@@H]2C)c2cc(F)c(-c3c(O)cccc3F)nc21 ; C#CCCNC(=O)C(=C)CN1CCN(c2nc(=O)n(-c3c(C)ccnc3C(C)C)c3nc(-c4c(O)cccc4F)c(F)cc23)[C@@H](C)C1]

To a stirred solution of amg-510 amine (2.5 mg, 0.005 mmol) in anhydrous DCM (1 mL), DIPEA (1.6 uL, 0.01 mmol) and compound 2a (2.9 mg, 0.01 mmol) were added at 25° C. The reaction mixture was stirred at room temperature for 12 h. After completion of the reaction (as monitored by LC-MS), CH₂Cl₂ was concentrated in vacuo to obtain the crude carboxylic acid. the crude product was purified by High Pressure Liquid Chromatography (HPLC) using water:ACN (0.1% formic acid) solvent gradient to afford white solid 7h in 1 mg (31.0% yield).

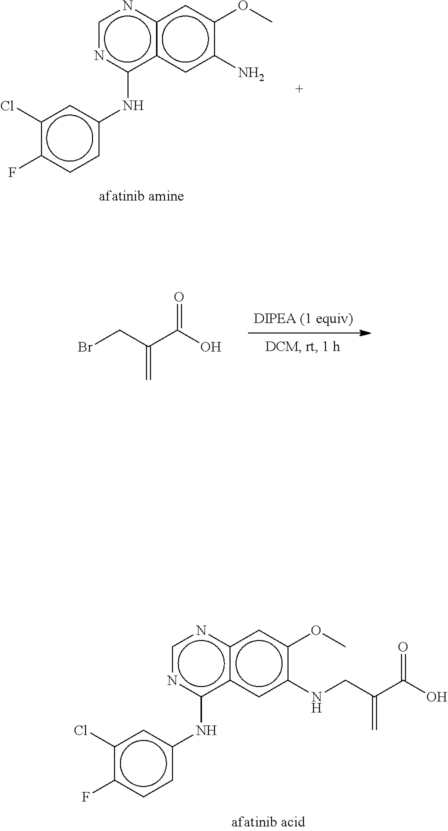

To a stirred solution of afatinib amine (32 mg, 0.1 mmol) in anhydrous DCM (2 mL), DIPEA (17.8 uL, 0.1 mmol) and 2-(bromomethyl)acrylic acid (16.1 mg, 0.1 mmol) were added at 25° C. The reaction mixture was stirred at room temperature for 1 h. After completion of the reaction (as monitored by LC-MS), CH₂Cl₂ was concentrated in vacuo to obtain the crude carboxylic acid. the crude product was purified by High Pressure Liquid Chromatography (HPLC) using water:ACN (0.1% formic acid) solvent gradient to afford white solid of afatinib carboxylic acid in 16 mg (42% yield).

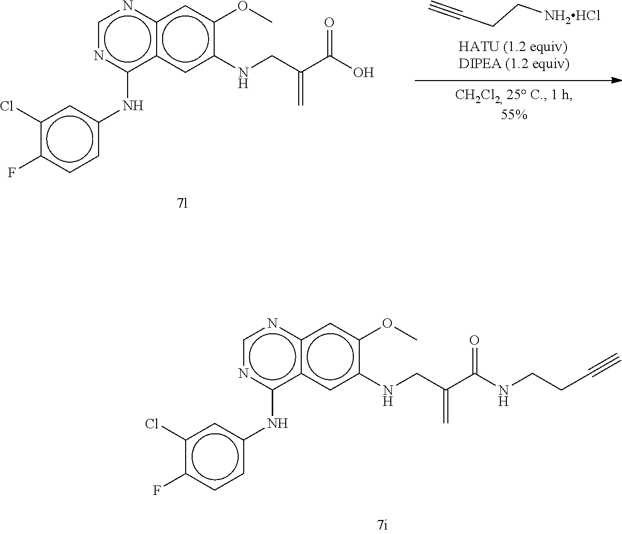

To a solution of carboxylic acid (20 mg, 0.05 mmol) in CH₂Cl₂ (1 mL), HATU (23 mg, 0.06 mmol), DIPEA (11 uL, 0.06 mmol) and N-methylprop-2-yn-1-amine hydrochloride (6.3 mg, 0.06 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 7i in 12.4 mg (55% yield).

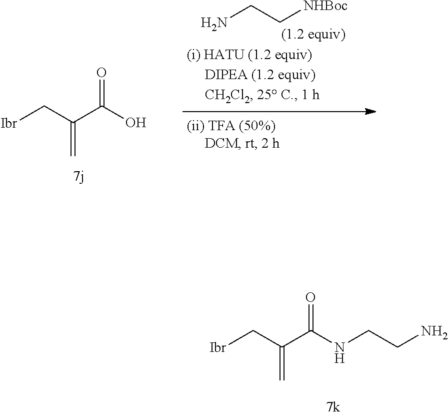

To a solution of carboxylic acid 7j (188 mg, 0.4 mmol) in CH₂Cl₂ (5 mL), HATU (182 mg, 0.48 mmol), DIPEA (85 uL, 0.48 mmol) and N-boc ethelene diamine (96 mg, 0.6 mmol) were added at 25° C. The reaction mixture was stirred at room temperature for 2 h. After completion of the reaction (as monitored by LC-MS), water was added. the aqueous layer was extracted with CH₂Cl₂ (3×3 mL). The organic layer was concentrated in vacuo and the crude product was dissolved in 50% TFA in dichlormethane and allowed to stir at 25° C. for 2 h. The reaction mixture was concentrated and purified by using flash column chromatography in hexane: ethyl acetate:methanol solvent system to get pure carboxylic acid 7m (yield xxx) in 94 mg (46.0% yield).

### Synthesis of 7m:

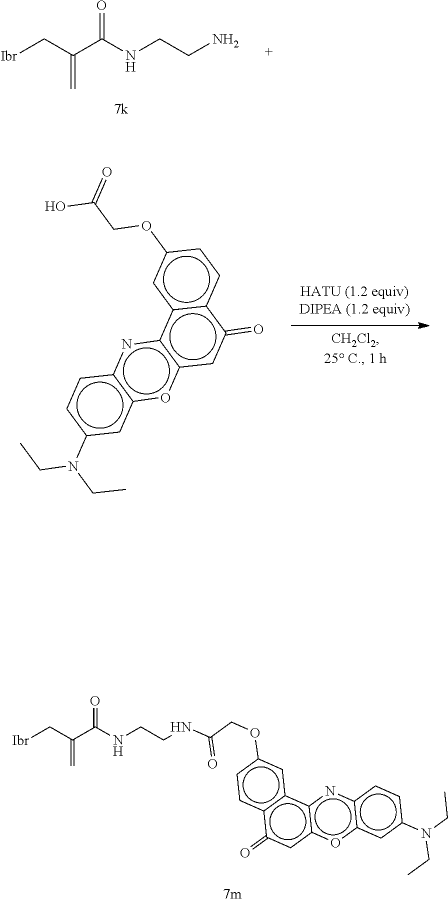

To a solution of carboxylic acid (19 mg, 0.05 mmol) in CH₂Cl₂ (1 mL), HATU (23 mg, 0.12 mmol), DIPEA (11 uL, 0.12 mmol) and amine 7k (26 mg, 0.12 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 7m in 9 mg (20% yield).

### Synthesis of 7n

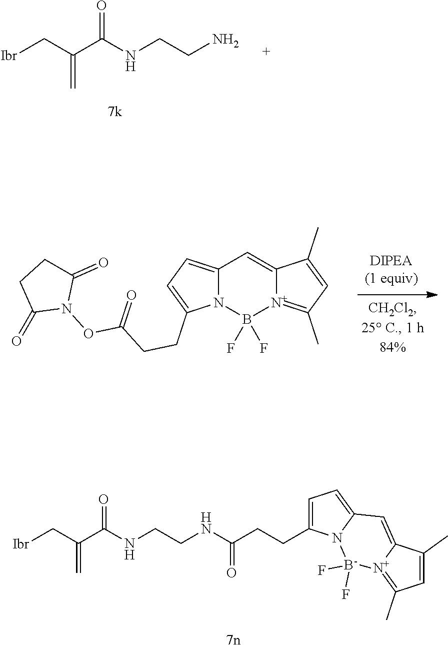

To a stirred solution of amine 7k (6.4 mg, 0.0125 mmol) in CH₂Cl₂ (0.5 mL), BODIPY NHS ester (4.9 mg, 0.0125 mmol), DIPEA (2.2 μL, 0.025 mmol) were added at 25° C. The reaction mixture was stirred at room temperature for 1 h. After completion of the reaction (as monitored by LC-MS), the reaction mixture was concentrated under vacuo and the crude product was purified by preparative HPLC using water:ACN (0.1% TFA) solvent gradient to afford 7n as bright yellow color solid in 6.2 mg (yield=64%).

¹H NMR (400 MHz, CD₃OD): δ 1.32-1.37 (m, 2H), 1.85-2.02 (br. s., 2H), 2.21 (d, J=7.5 Hz, 2H), 2.28 (s, 3H), 2.46 (br. s., 4H), 3.10 (t, J=7.7 Hz, 2H), 3.14 (dt, J=3.3, 1.7 Hz, 1H), 3.49 (dt, J=3.2, 1.7 Hz, 1H), 3.58-3.73 (m, 3H), 3.77 (s, 1H), 3.95-4.03 (m, 1H), 4.03-4.11 (m, 1H), 4.15 (d, J=12.8 Hz, 1H), 5.36-5.46 (m, 1H), 6.02 (s, 1H), 6.21 (s, 2H), 6.25 (br. s., 1H), 6.96 (br. s., 1H), 7.11 (d, J=7.7 Hz, 2H), 7.18 (d, J=9.5 Hz, 2H), 7.20-7.25 (m, 1H), 7.35-7.50 (m, 3H), 7.52-7.64 (m, 1H), 7.80-7.92 (m, 1H), 8.38 (br. s., 1H); ¹³C NMR (100 MHz, CD₃OD): δ 11.3, 15.0, 20.0, 25.7, 28.4, 36.1, 41.0, 52.1, 53.2, 55.5, 61.2, 117.5, 120.0, 120.9, 121.6, 125.5, 125.9, 127.4, 129.7, 131.3, 132.0, 134.9, 136.7, 140.5, 146.2, 153.5, 154.2, 157.9, 158.4, 160.7, 161.6, 169.3, 175.2; HR-MS (m/z): Calculated for C₄₂H₄₆BF₂N₁₀O₃[M+H]⁺: 787.3815; Found [M+H]⁺: 787.3828.

### Synthesis of 8a and 8b:

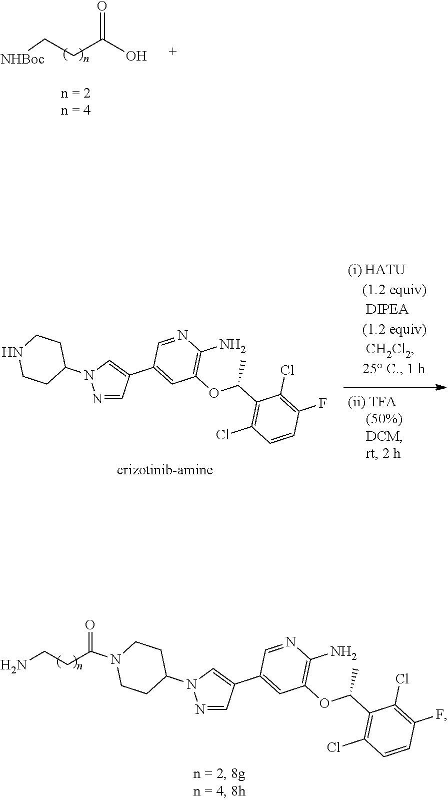

To a solution of carboxylic acid (0.1 mmol) in CH₂Cl₂ (5 mL), HATU (45.6 mg, 0.12 mmol), N,N-Diisopropylethylamine (DIPEA) (21.5 uL, 0.12 mmol) and crizotinib-amine (45 mg, 0.1 mmol) were added at 25° C. The reaction mixture was stirred at room temperature for 2 h. After completion of the reaction (as monitored by LC-MS), water was added. the aqueous layer was extracted with CH₂Cl₂ (3×3 mL). The organic layer was concentrated in vacuo and the crude product was dissolved in 50% TFA in dichlormethane and allowed to stir at 25° C. for 2 h. The reaction mixture was concentrated and purified by High Pressure Liquid Chromatography (HPLC) using water:ACN (0.1% formic acid) solvent gradient to afford white solid 8g/8h.

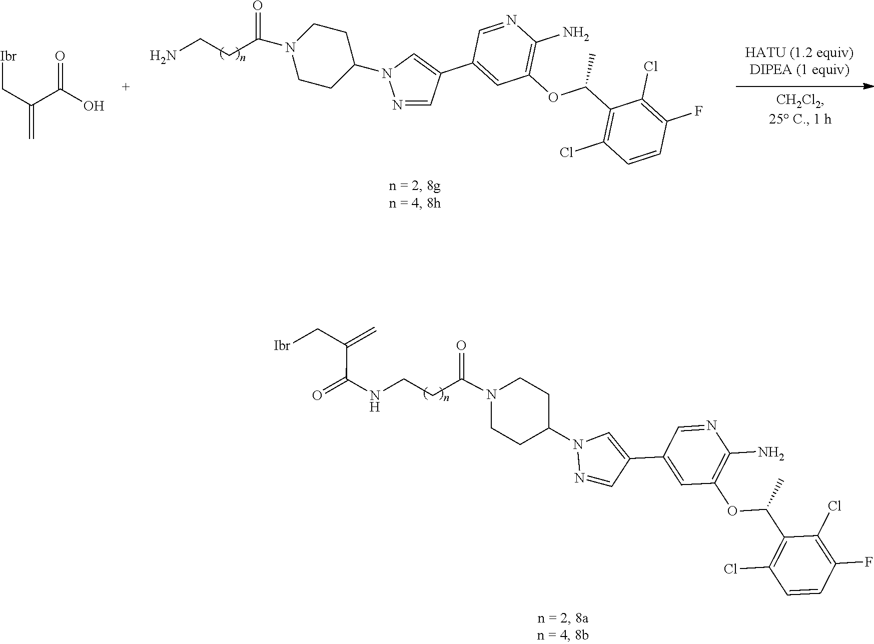

To a solution of carboxylic acid (23.5 mg, 0.05 mmol) in CH₂Cl₂ (1 mL), HATU (23 mg, 0.12 mmol), DIPEA (11 uL, 0.12 mmol) and 8g/8h (0.05 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 8a/8b.

### Synthesis of 8c and 8d:

The synthesis of 8c and 8d has been carried out using the same scheme and procedure as used for the synthesis of 8a and 8b where crizotinib-amine was replaced by afatinib-amine.

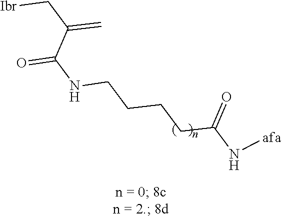

### Synthesis of 8e:

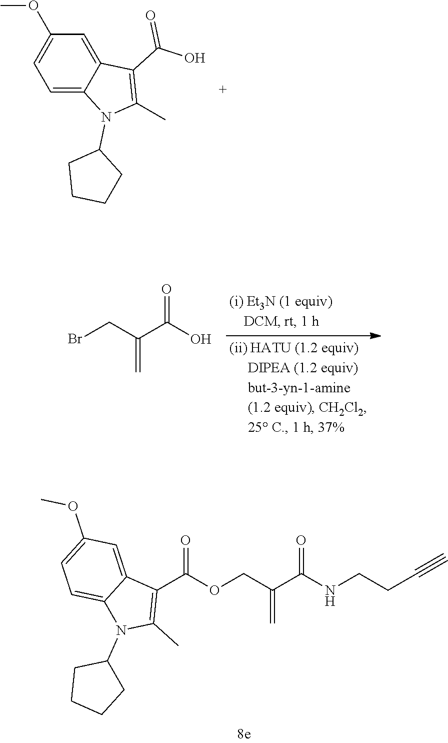

To a stirred solution of indole carboxylic acid (27.3 mg, 0.1 mmol) in anhydrous DCM (1 mL), Et₃N (6.9 uL, 0.1 mmol) and 2-(bromomethyl)acrylic acid (16.1 mg, 0.1 mmol) were added at 25° C. The reaction mixture was stirred at room temperature for 1 h. After completion of the reaction (as monitored by LC-MS), CH₂Cl₂ was concentrated in vacuo to obtain the crude carboxylic acid.

To a solution of crude carboxylic acid in CH₂Cl₂ (0.5 mL), HATU (45.6 mg, 0.12 mmol), DIPEA (21.5 uL, 0.12 mmol) and but-3-yn-1-amine hydrochloride (12.6 mg, 0.12 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 8e in 9.8 mg (37% yield).

### Synthesis of 8f:

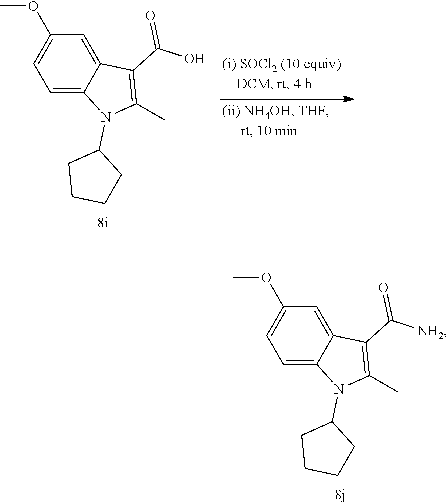 [SMILES: COc1ccc2c(c1)c(C(=O)O)c(C)n2C1CCCC1 ; COc1ccc2c(c1)c(C(N)=O)c(C)n2C1CCCC1]

To a stirred solution of indole carboxylic acid (27.3 mg, 0.1 mmol) in anhydrous DCM (1 mL) SOC₂ (73 uL, 1 mmol) was added at 25° C. The reaction mixture was stirred at room temperature for 4 h. After completion of the reaction (as monitored by LC-MS), CH₂Cl₂ was concentrated in vacuo to obtain the crude acid chloride. The crude carboxylic acid was dissolved in 1 mL THF and poured in 5 mL solution of ammonium hydroxide at 0° C. and allowed it to stir for 10 min. The reaction mixture was concentrated in vacuo and the crude product was purified by High Pressure Liquid Chromatography (HPLC) using water:ACN (0.1% formic acid) solvent gradient to afford white solid 8j in 60% yield (14 mg).

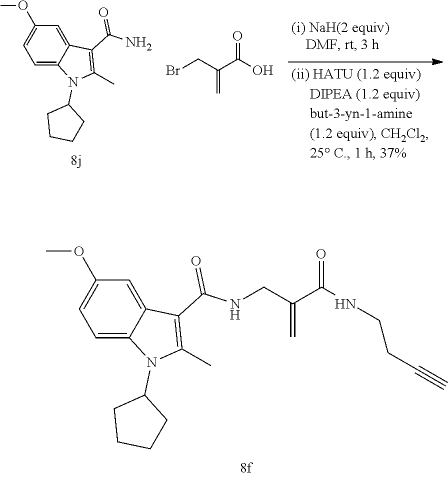

To a stirred solution of 8j (14 mg, 0.05 mmol) in anhydrous DMF (0.5 mL), NaH (6.9 uL, 0.1 mmol) and 2-(bromomethyl)acrylic acid (16.1 mg, 0.1 mmol) were added at 25° C. under N₂ atmosphere. The reaction mixture was stirred at room temperature for 3 h. After completion of the reaction (as monitored by LC-MS), The organic layer was concentrated in vacuo and the crude product was purified by High Pressure Liquid Chromatography (HPLC) using water:ACN (0.1% formic acid) solvent gradient to afford white solid carboxylic acid.

To a solution of carboxylic acid (5 mg, 0.02 mmol) in CH₂Cl₂ (0.5 mL), HATU (11.4 mg, 0.03 mmol), DIPEA (5 uL, 0.03 mmol) and but-3-yn-1-amine hydro chloride (3.1 mg, 0.03 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 8f in 9.8 mg (37% yield).

### Example 2

### Synthesis of Model Compounds of this Invention

### N-benzyl-2-(hydroxymethyl)acrylamide (1k)

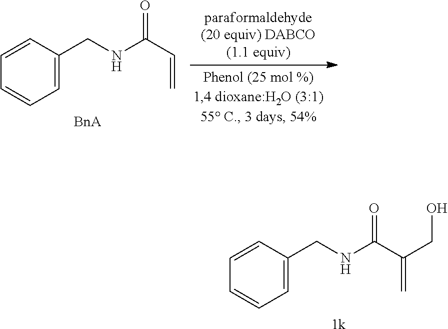

To a stirred solution of acrylamide (644 mg, 4 mmol) in 1,4-dioxane:H₂O (3:1 v/v, 12 mL) were added DABCO (492.8 mg, 4.4 mmol), phenol (87 uL, 1 mmol) and paraformaldehyde (2.4 g, 80 mmol) at 25° C. The reaction mixture was stirred at room temperature for 3d. After completion of the reaction (as monitored by LC-MS), 1,4 dioxane was concentrated in vacuo and the aqueous layer was extracted with EtOAc (3×30 mL). The organic layer was concentrated in vacuo and the crude product was purified by column chromatography over silica gel using EtOAc/Pet. ether as eluent to give pure alcohol as white solid (412 mg, yield=54%) N-benzyl-2-(bromomethyl)acrylamide (1l)

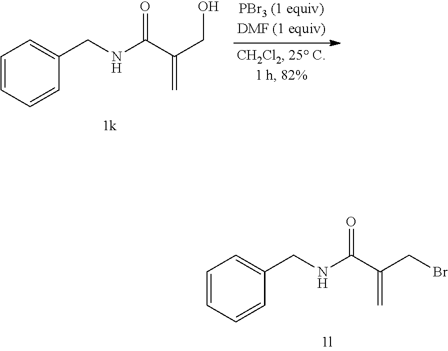

To a stirred solution of alcohol (191 mg, 1 mmol) in CH₂Cl₂ (5 mL) was added PBr₃ (105 uL, 1.1 mmol) and DMF (77 uL, 1 mmol) at 0° C. under N₂ atm. The reaction mixture was stirred at room temperature for 1 h under and quenched with H₂O (5 mL) at 0° C. The aqueous layer was extracted with CH₂Cl₂ (3×8 mL) concentrated in vacuo. The crude product was purified by column chromatography over silica gel using EtOAc/Pet. ether as eluent to give pure bromo compound as white solid (207 mg, yield=82%).

### N-benzylmethacrylamide (1a)

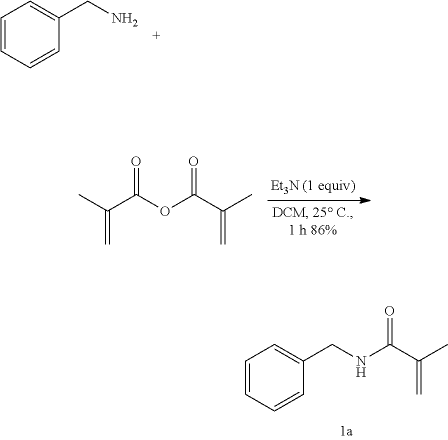

To a stirred solution of benzyl amine (10.6 mg, 0.1 mmol) in anhydrous DCM (0.5 mL) Et₃N (13.9 uL, 0.1 mmol) and methacrylic anhydride (15.4 uL, 0.1 mmol) were added at 25° C. The reaction mixture was stirred at room temperature for 1 h. After completion of the reaction (as monitored by LC-MS), CH₂Cl₂ was concentrated in vacuo. The crude product was purified by High Pressure Liquid Chromatography (HPLC) using water:ACN (0.1% formic acid) solvent gradient to afford white solid 1a in 15.1 mg (86.2% yield).

¹H NMR (500 MHz, CDCl₃) δ 1.99 (s, 3H), 4.51 (d, J=5.6 Hz, 2H), 5.36 (s, 1H), 5.73 (s, 1H), 6.12 (br. s., 1H), 7.27-7.38 (m, 5H); ¹³C NMR (125 MHz, CDCl₃) δ 18.7, 43.8, 119.7, 127.5, 127.8, 128.7, 138.2, 139.9, 168.3.

### N-benzyl-2-(piperidin-1-ylmethyl)acrylamide (1b)

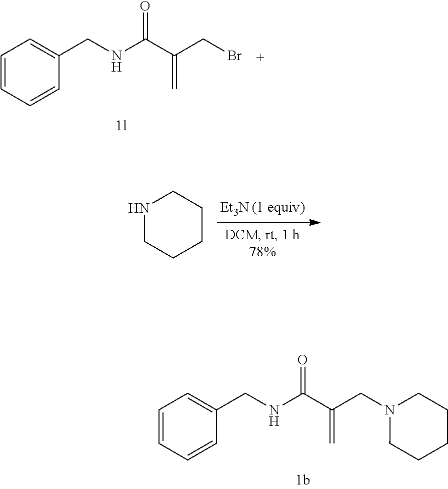 [SMILES: C=C(CBr)C(=O)NCc1ccccc1 ; C1CCNCC1 ; C=C(CN1CCCCC1)C(=O)NCc1ccccc1]

To a stirred solution of piperidine (9.9 uL, 0.1 mmol) in anhydrous DCM (0.5 mL) were added Et₃N (13.9 uL, 0.1 mmol) and N-benzyl-2-(bromomethyl)acrylamide (1l) (25.4 mg, 0.1 mmol) at 25° C. The reaction mixture is allowed to stir at room temperature for one hour. After completion of the reaction (as monitored by LC-MS, CH₂Cl₂ was concentrated in vacuo. The crude product was purified by High-Pressure Liquid Chromatography (HPLC) using water: ACN (0.1% formic acid) solvent gradient to afford white solid ab in 20.1 mg (78% yield).

¹H NMR (400 MHz, CDCl₃) δ 1.56 (br. s., 1H) 1.91 (br. s., 3H) 2.02 (d, J=11.7 Hz, 2H) 2.81 (t, J=11.3 Hz, 2H) 3.62 (d, J=11.7 Hz, 2H) 4.05 (s, 2H) 4.69 (d, J=5.9 Hz, 3H) 6.21 (s, 1H) 6.35 (s, 1H) 7.35-7.59 (m, 5H) 7.78 (br. s., 1H); ¹³C NMR (100 MHz, CDCl₃) δ 21.7, 22.7, 43.9, 53.2, 56.7, 127.6, 127.8, 128.7, 129.4, 133.8, 137.7, 167.0

### N-benzyl-2-((methyl(phenyl)amino)methyl)acrylamide (1c)

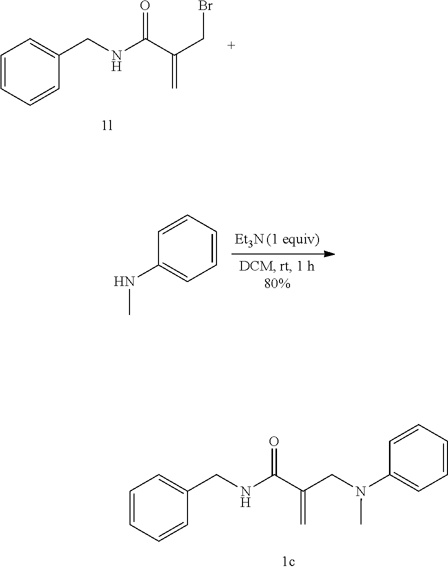

To a stirred solution of N-methyl aniline (10.7 mg, 0.1 mmol) in anhydrous DCM (0.5 mL) was added Et₃N (13.9 uL, 0.1 mmol) and N-benzyl-2-(bromomethyl)acrylamide (1l) (25.4 mg, 0.1 mmol) were added at 25° C. The reaction mixture was stirred at room temperature for 1 h. After completion of the reaction (as monitored by LC-MS, CH₂Cl₂ was concentrated in vacuo. The crude product was purified by High Pressure Liquid Chromatography (HPLC) using water:ACN (0.1% formic acid) solvent gradient to afford white solid 1c in 22.4 mg (80% yield).

¹H NMR (500 MHz, CDCl₃) δ 3.06 (s, 3H) 4.28 (s, 2H) 4.42 (d, J=5.64 Hz, 2H) 5.76 (s, 1H) 6.03 (s, 1H) 7.13-7.19 (m, 1H) 7.22 (t, J=8.05 Hz, 4H) 7.30 (d, J=7.02 Hz, 1H) 7.32 (s, 1H) 7.34-7.39 (m, 2H) 8.63 (br. s., 2H): ¹³C NMR (125 MHz, CDCl₃) δ 41.7, 43.7, 57.8, 118.2, 124.7, 125.8, 127.6, 127.7, 128.7, 129.8, 136.6, 137.5, 145.0, 160.8, 161.1, 167.3.

### N-benzyl-2-((benzylamino)methyl)acrylamide (1d)

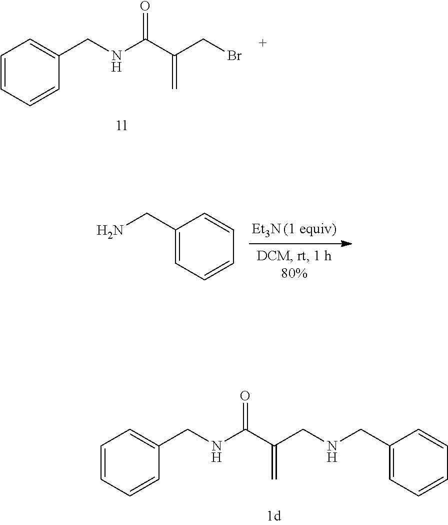 [SMILES: C=C(CBr)C(=O)NCc1ccccc1 ; NCc1ccccc1 ; C=C(CNCc1ccccc1)C(=O)NCc1ccccc1]

To a stirred solution of benzyl amine (10.7 mg, 0.1 mmol) in anhydrous DCM (0.5 mL) were added Et₃N (13.9 uL, 0.1 mmol) and N-benzyl-2-(bromomethyl)acrylamide (1l) (25.4 mg, 0.1 mmol) at 25° C. The reaction mixture was stirred at room temperature for 1 h. After completion of the reaction (as monitored by LC-MS), CH₂Cl₂ was concentrated in vacuo. The crude product was purified by High Pressure Liquid Chromatography (HPLC) using water:ACN (0.1% formic acid) solvent gradient to afford white solid 1d in 22.5 mg (80% yield).

¹H NMR (500 MHz, CDCl₃) δ 3.84 (s, 2H), 4.20 (s, 2H), 4.46 (d, J=5.5 Hz, 2H), 5.86 (s, 1H), 6.00 (s, 1H), 6.98 (br. s., 1H), 7.28-7.39 (m, 6H), 7.40-7.49 (m, 4H); ¹³C NMR (125 MHz, CDCl₃) δ 43.9, 49.3, 51.0, 127.0, 127.9, 128.3, 128.9, 129.4, 129.8, 129.9, 130.1, 133.5, 137.0, 167.4.

### N-benzyl-2-(phenoxymethyl)acrylamide (1e)

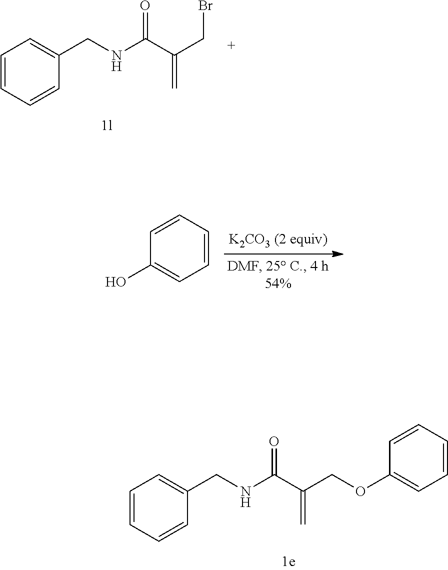 [SMILES: C=C(CBr)C(=O)NCc1ccccc1 ; Oc1ccccc1 ; C=C(COc1ccccc1)C(=O)NCc1ccccc1]

To a stirred solution of phenol (9.4 mg, 0.1 mmol) in anhydrous DMF (0.5 mL) was added K₂CO₃ (27.6 mg, 0.2 mmol) at 25° C. After stirring for 5 min, N-benzyl-2-(bromomethyl)acrylamide (1l) (25.4 mg, 0.1 mmol) was added at 25° C. under N₂ atm. The reaction mixture was stirred at room temperature for 4 h under N₂ atm and quenched with H₂O (2 mL). The aqueous layer was extracted with EtOAc (3×3 mL). The organic layer was washed with brine and concentrated in vacuo. The crude product was purified by High Pressure Liquid Chromatography (HPLC) using water:ACN (0.1% formic acid) solvent gradient to afford white solid 1e in 14.4 mg (53.9% yield).

¹H NMR (500 MHz, CDCl₃) δ 4.56 (d, J=5.64 Hz, 2H) 4.82 (s, 3H) 5.74 (s, 1H) 6.10 (s, 1H) 6.67 (br. s., 1H) 6.92 (d, J=7.98 Hz, 2H) 7.00 (t, J=7.29 Hz, 1H) 7.26-7.29 (m, 1H) 7.29-7.33 (m, 4H) 7.33-7.39 (m, 2H); ¹³C NMR (125 MHz, CDCl₃) δ 43.7, 67.9, 114.9, 121.6, 123.1, 127.6, 127.7, 128.7, 129.6, 137.9, 139.2, 157.7, 166.3.

### N-benzyl-2-((4-nitrophenoxy)methyl)acrylamide (1f)

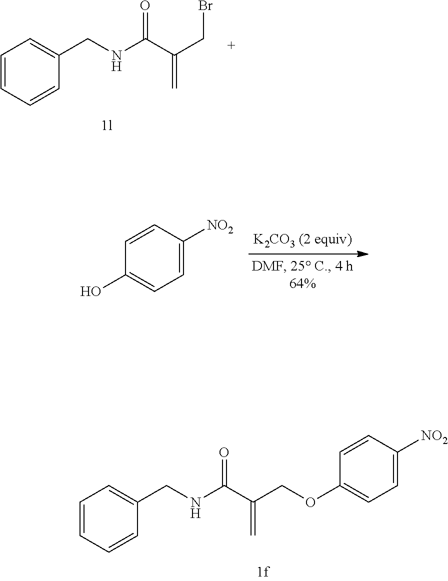 [SMILES: C=C(CBr)C(=O)NCc1ccccc1 ; O=[N+]([O-])c1ccc(O)cc1 ; C=C(COc1ccc([N+](=O)[O-])cc1)C(=O)NCc1ccccc1]

To a stirred solution of 4-nitrophenol (13.9 mg, 0.1 mmol) in anhydrous DMF (0.5 mL) was added K₂CO₃ (27.6 mg, 0.2 mmol) at 25° C. After stirring for 5 min, N-benzyl-2-(bromomethyl)acrylamide (1l) (25.4 mg, 0.1 mmol) was added at 25° C. under N₂ atm. The reaction mixture was stirred at room temperature for 4 h under N₂ atm and quenched with H₂O (2 mL). The aqueous layer was extracted with EtOAc (3×3 mL). The organic layer was washed with brine and concentrated in vacuo. The crude product was purified by High Pressure Liquid Chromatography (HPLC) using water:ACN (0.1% formic acid) solvent gradient to afford white solid if in 20.2 mg (64.7% yield).

¹H NMR (500 MHz, CDCl₃) δ 4.54 (d, J=5.8 Hz, 2H), 4.92 (s, 2H), 5.76 (s, 1H), 5.96 (s, 1H), 6.46 (br. s., 1H), 7.00 (d, J=9.2 Hz, 2H), 7.29-7.40 (m, 5H), 8.19 (d, J=9.2 Hz, 2H): ¹³C NMR (125 MHz, CDCl₃) δ 43.7, 67.7, 114.8, 121.2, 125.9, 127.7, 128.8, 137.6, 139.0, 141.9, 162.9, 166.1.

### N-benzyl-2-(((2-oxo-2H-chromen-7-yl)oxy)methyl)acrylamide (1g)

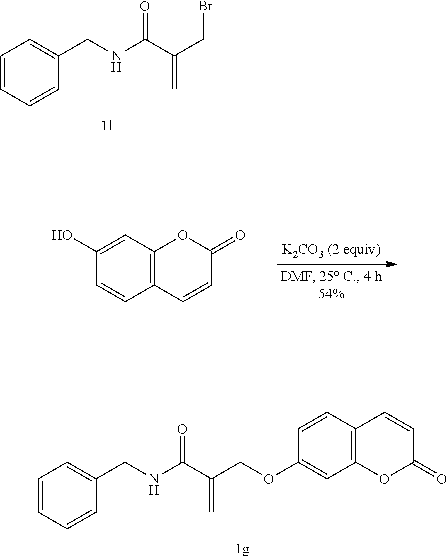 [SMILES: C=C(CBr)C(=O)NCc1ccccc1 ; O=c1ccc2ccc(O)cc2o1 ; C=C(COc1ccc2ccc(=O)oc2c1)C(=O)NCc1ccccc1]

To a stirred solution of 7-hydroxy-2H-chromen-2-one (16.2 mg, 0.1 mmol) in anhydrous DMF (0.5 mL) was added K₂CO₃ (27.6 mg, 0.2 mmol) at 25° C. After stirring for 5 min, N-benzyl-2-(bromomethyl)acrylamide (1l) (25.4 mg, 0.1 mmol) was added at 25° C. under N₂ atm. The reaction mixture was stirred at room temperature for 4 h under N₂ atm and quenched with H₂O (2 mL). The aqueous layer was extracted with EtOAc (3×3 mL). The organic layer was washed with brine and concentrated in vacuo. The crude product was purified by High Pressure Liquid Chromatography (HPLC) using water:ACN (0.1% formic acid) solvent gradient to afford white solid 1g in 18.1 mg (54% yield).

¹H NMR (500 MHz, CDCl₃) δ 4.54 (d, J=5.6 Hz, 2H), 4.88 (s, 2H), 5.75 (s, 1H), 6.00 (s, H), 6.24 (d, J=9.5 Hz, 1H), 6.58 (br. s., 1H), 6.83 (d, J=2.2 Hz, 1H), 6.85-6.88 (m, 1H), 7.28-7.32 (m, 3H), 7.34 (d, J=7.2 Hz, 2H), 7.37 (d, J=8.7 Hz, 1H), 7.62 (d, J=9.5 Hz, 1H); ¹³C NMR (125 MHz, CDCl₃) δ 43.6, 67.8, 102.2, 112.6, 113.4, 121.8, 127.6, 127.7, 128.7, 128.8, 137.7, 139.0, 143.2, 155.6, 161.0, 166.0.

### N-benzyl-2-((benzyloxy)methyl)acrylamide (1h)

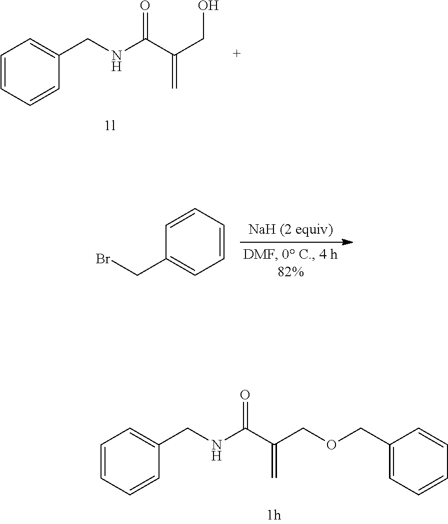 [SMILES: C=C(CO)C(=O)NCc1ccccc1 ; BrCc1ccccc1 ; C=C(COCc1ccccc1)C(=O)NCc1ccccc1]

To a stirred solution of N-benzyl-2-(hydroxymethyl)acrylamide (1h) (19.1 mg, 0.1 mmol) in anhydrous DMF (0.5 mL) was added NaH (8 mg (60% in mineral oil), 0.2 mmol)) at 0° C. After stirring for 5 min, benzyl bromide (13 uL, 0.11 mmol) was added at 0° C. under N₂ atm. The reaction mixture was stirred at room temperature for 4 h under N₂ atm and quenched with H₂O (2 mL) at 0° C. The aqueous layer was extracted with EtOAc (3×3 mL). The organic layer was washed with brine and concentrated in vacuo. The crude product was purified by High Pressure Liquid Chromatography (HPLC) using water:ACN (0.1% formic acid) solvent gradient to afford white solid 1h in 18.4 mg (65.4% yield).

¹H NMR (500 MHz, CDCl₃) δ 4.31 (s, 2H), 4.46-4.58 (m, 4H), 5.61 (s, 1H), 6.30 (d, J=1.2 Hz, 1H), 7.18-7.25 (m, 2H), 7.25-7.38 (m, 10H). ¹³C NMR (125 MHz, CDCl₃) δ 43.6, 70.5, 72.0, 125.7, 127.4, 127.8, 128.0, 128.0, 128.6, 128.7, 137.0, 138.1, 138.6, 166.3.

### 2-(benzylcarbamoyl)allyl benzoate (1i)

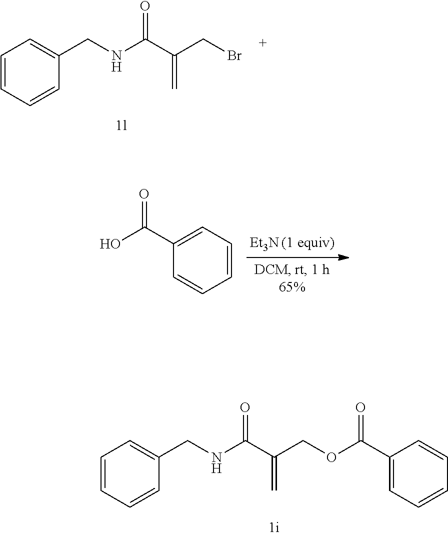 [SMILES: C=C(CBr)C(=O)NCc1ccccc1 ; O=C(O)c1ccccc1 ; C=C(COC(=O)c1ccccc1)C(=O)NCc1ccccc1]

To a stirred solution of benzoic acid (12.2 mg 0.1 mmol) in anhydrous DCM (0.5 mL) was added Et₃N (13.9 uL, 0.1 mmol) and N-benzyl-2-(bromomethyl)acrylamide (1l) (25.4 mg, 0.1 mmol) were added at 25° C. The reaction mixture was stirred at room temperature for 1 h. After completion of the reaction as monitored by LC-MS, CH₂Cl₂ was concentrated in vacuo. The crude product was purified by High Pressure Liquid Chromatography (HPLC) using water:ACN (0.1% formic acid) solvent gradient to afford white solid 1i in 19.4 mg (65.7% yield).

¹H NMR (500 MHz, CDCl₃) δ 2.47 (br. s., 1H), 4.55 (s, 2H), 5.12 (s, 2H), 5.74 (s, 1H), 6.05 (s, 1H), 6.56 (br. s., 1H), 7.30 (s, 3H), 7.43 (t, J=7.77 Hz, 2H), 7.58 (t, J=7.43 Hz, 1H), 7.99 (d, J=7.70 Hz, 1H); ¹³C NMR (125 MHz, CDCl₃) δ 43.7, 63.8, 123.2, 127.5, 127.7, 128.4, 128.7, 129.5, 129.6, 133.2, 137.8, 139.1, 166.0.

### 2-(benzylcarbamoyl)allyl (4-nitrophenyl) carbonate (1j)

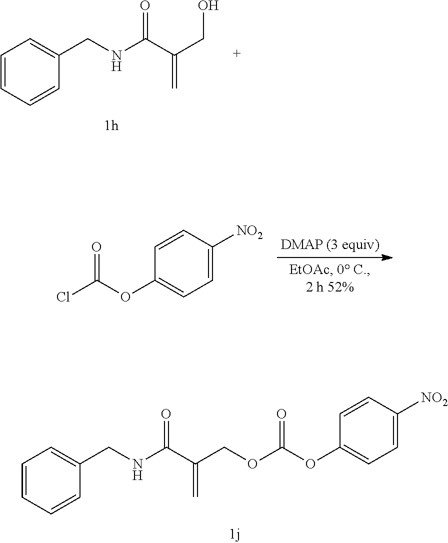 [SMILES: C=C(CO)C(=O)NCc1ccccc1 ; O=C(Cl)Oc1ccc([N+](=O)[O-])cc1 ; C=C(COC(=O)Oc1ccc([N+](=O)[O-])cc1)C(=O)NCc1ccccc1]

To a stirred solution of N-benzyl-2-(hydroxymethyl)acrylamide (1h) (19.2 mg, 0.1 mmol) in anhydrous ethyl acetate (0.5 mL) were added 4-nitrophenyl chloroformate (80.4 mg, 0.4 mmol) and 4-Dimethylaminopyridine (25.4 mg, 0.4 mmol) at 25° C. The reaction mixture was allowed to stir at room temperature for 1 h. After completion of the reaction as monitored by LC-MS, the reaction mixture is quenched with 0.1 N HCl (2 mL) and the aqueous layer was extracted with EtOAc (3×3 mL). The organic layer was washed with brine and concentrated in vacuo. The crude product was purified by High Pressure Liquid Chromatography (HPLC) using water:ACN (0.1% formic acid) solvent gradient to afford white solid 1j in 18.6 mg (52.2% yield).

¹H NMR (400 MHz, CDCl₃) δ 4.66 (d, J=5.5 Hz, 2H) 5.17 (s, 2H) 5.89 (s, 1H) 6.07 (s, 1H) 6.41 (br. s., 1H) 7.29-7.54 (m, 7H) 8.38 (d, J=9.2 Hz, 2H); ¹³C NMR (100 MHz, CDCl₃) δ 43.9, 67.9, 121.7, 123.0, 125.3, 127.8, 127.9, 128.9, 138.4, 152.1, 155.3, 165.7.

### N-(but-3-yn-1-yl)-2-(((2-oxo-2H-chromen-7-yl)oxy)methyl)acrylamide (2a)

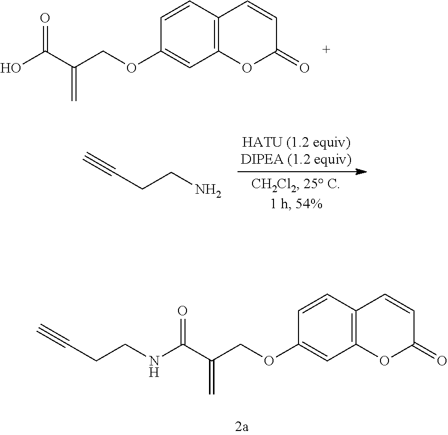

To a solution of 2-(((2-oxo-2H-chromen-7-yl)oxy)methyl)acrylic acid (synthesis is described in Example 1.

(24.7 mg, 0.1 mmol) in CH₂Cl₂ (1 mL), HATU (45.6 mg, 0.12 mmol), DIPEA (21.5 uL, 0.12 mmol) and but-3-yn-1-amine hydro chloride (12.6 mg, 0.12 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 2a in 16.14 mg (54% yield).

¹H NMR (400 MHz, CDCl₃) δ 2.01 (t, J=2.5 Hz, 1H), 2.48 (td, J=6.4, 2.6 Hz, 2H), 3.52 (q, J=6.3 Hz, 2H), 4.86 (s, 2H), 5.76 (s, 1H), 6.03 (s, 1H), 6.27 (d, J=9.5 Hz, 1H), 6.57 (br. s., 1H), 6.85-6.95 (m, 2H), 7.40 (d, J=8.4 Hz, 1H), 7.64 (d, J=9.5 Hz, 1H); ¹³C NMR (100 MHz, CDCl₃) δ 19.3, 38.0, 67.9, 70.3, 81.3, 102.3, 112.7, 113.1, 113.6, 122.3, 128.9, 138.8, 143.3, 155.7, 161.0, 166.2.

### N-(but-3-yn-1-yl)-2-((methyl(phenyl)amino)methyl)acrylamide (2b)

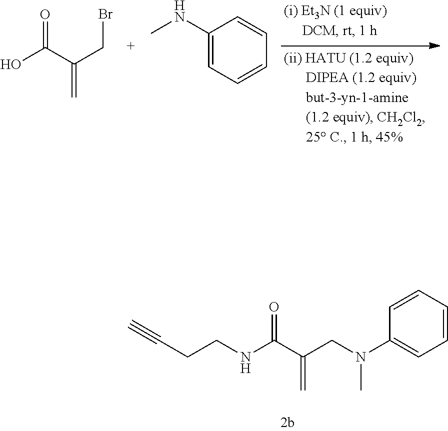

To a stirred solution of N-methyl aniline (10.3 mg, 0.1 mmol) in anhydrous DCM (1 mL), Et₃N (6.9 uL, 0.1 mmol) and 2-(bromomethyl)acrylic acid (16.1 mg, 0.1 mmol) were added at 25° C. The reaction mixture was stirred at room temperature for 1 h. After completion of the reaction (as monitored by LC-MS), CH₂Cl₂ was concentrated in vacuo to obtain the crude carboxylic acid.

To a solution of crude carboxylic acid in CH₂Cl₂ (1 ML), HATU (45.6 mg, 0.12 mmol), DIPEA (21.5 uL, 0.12 mmol) and but-3-yn-1-amine hydro chloride (12.6 mg, 0.12 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 2b in 10.9 mg (45.0% yield over two steps).

¹H NMR (400 MHz, CDCl₃) δ 1.98 (t, J=2.6 Hz, 1H), 2.26-2.36 (m, 2H), 3.18 (s, 3H), 3.31 (d, J=6.2 Hz, 2H), 4.37 (s, 2H), 6.15 (s, 1H), 6.27 (s, 1H), 6.86 (br. s., 1H), 7.40 (d, J=7.3 Hz, 1H), 7.43-7.51 (m, 2H), 7.63 (d, J=7.9 Hz, 2H); ¹³C NMR (125 MHz, CDCl₃) δ 19.1, 38.2, 43.0, 57.9, 70.3, 81.0, 120.0, 127.2, 128.0, 130.0, 135.3, 159.4, 167.1.

### 2-(but-3-yn-1-ylcarbamoyl)allyl benzoate (2c)

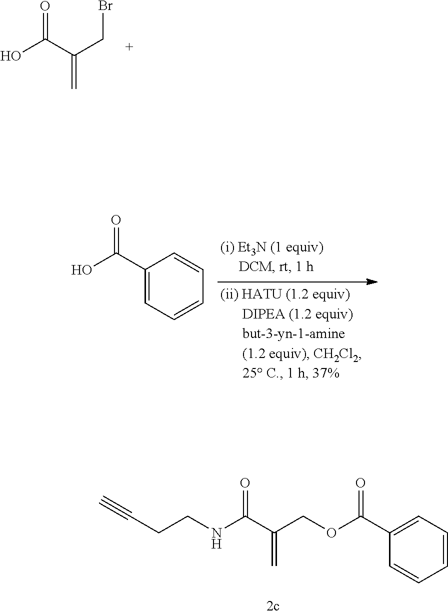

To a stirred solution of benzoic acid (10.7 mg, 0.1 mmol) in anhydrous DCM (1 mL), Et₃N (6.9 uL, 0.1 mmol) and 2-(bromomethyl)acrylic acid (16.1 mg, 0.1 mmol) were added at 25° C. The reaction mixture was stirred at room temperature for 1 h. After completion of the reaction (as monitored by LC-MS), CH₂Cl₂ was concentrated in vacuo to obtain the crude carboxylic acid.

To a solution of crude carboxylic acid in CH₂Cl₂ (0.5 mL), (1-[Bis(dimethylamino)methylene]-1H-1,2,3-triazolo[4,5-b]pyridinium 3-oxide hexafluorophosphate (HATU) (45.6 mg, 0.12 mmol), diisopropyl ethyl amine (DIPEA), 21.5 uL, 0.12 mmol) and but-3-yn-1-amine hydro chloride (12.6 mg, 0.12 mmol) were added at 25° C. The reaction mixture was stirred at room temperature for 1 h. After completion of the reaction (as monitored by LC-MS), water was added. the aqueous layer was extracted with CH₂Cl₂ (3×3 mL). The organic layer was concentrated in vacuo and the crude product was purified by High Pressure Liquid Chromatography (HPLC) using water:ACN (0.1% formic acid) solvent gradient to afford white solid 2c in 9.8 mg (37% yield).

¹H NMR (500 MHz, CDCl₃) δ 1.92 (t, J=2.6 Hz, 1H), 2.47 (td, J=6.3, 2.6 Hz, 2H), 3.52 (q, J=6.2 Hz, 2H), 5.11 (s, 2H), 5.76 (s, 1H), 6.07 (s, 1H), 6.52 (br. s., 1H), 7.42-7.50 (m, 2H), 7.55-7.64 (m, 1H), 8.07 (dd, J=8.3, 1.1 Hz, 2H); ¹³C NMR (125 MHz, CDCl₃) δ 19.3, 38.1, 63.8, 70.2, 81.3, 123.4, 128.5, 129.7, 133.3, 139.1, 166.2.

### (R)-3-(4-phenoxyphenyl)-1-(piperidin-3-yl)-1H-pyrazolo[3,4-d]pyrimidin-4-amine (Ibrutinib)

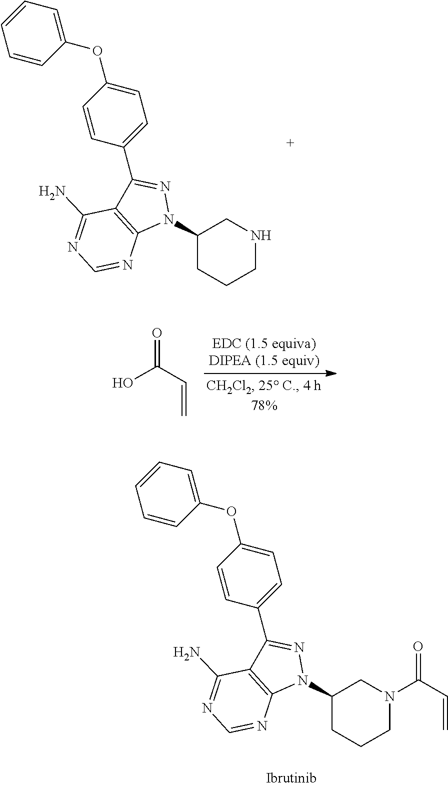

To a stirred solution of acrylic acid (1.02 mL, 15 mmol) in anhydrous CH₂Cl₂ (50 mL), N-(3-dimethylaminopropyl)-N′-ethylcarbodiimide hydrochloride (EDC·HCl) (2.88 g, 15 mmol), N,N-Diisopropylethylamine (2.60 mL, 15 mmol) and amine (3.87 g, 10 mmol) were added at 0° C. under N₂ atmosphere. The reaction mixture was allowed to stir at room temperature for 4 h. After completion (as monitored by LC-MS), of the reaction, H₂O (30 mL) was added. The organic layer was extracted with CH₂Cl₂ (3×50 mL) and concentrated in vacuo. The crude product was purified by column chromatography over silica gel using EtOAc/Pet. ether as eluent to give pure Ibrutinib as white solid (3.46 g, yield=78%)

### (R)-1-(3-(4-amino-3-(4-phenoxyphenyl)-1H-pyrazolo[3,4-d]pyrimidin-1-yl)piperidin-1-yl)-2-(hydroxymethyl)prop-2-en-1-one (3l)

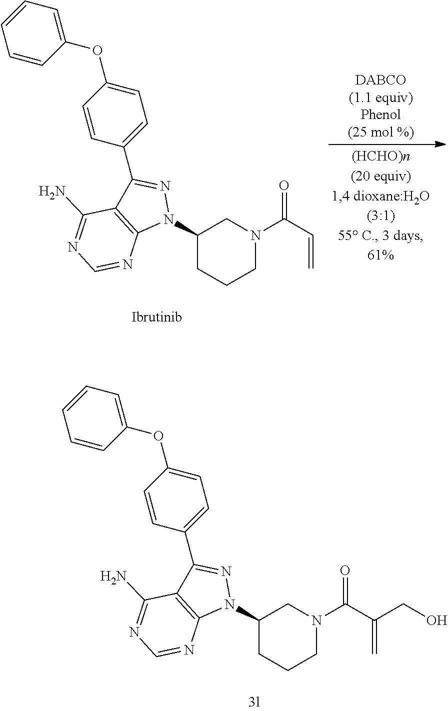 [SMILES: C=CC(=O)N1CCC[C@@H](n2nc(-c3ccc(Oc4ccccc4)cc3)c3c(N)ncnc32)C1 ; C=C(CO)C(=O)N1CCC[C@@H](n2nc(-c3ccc(Oc4ccccc4)cc3)c3c(N)ncnc32)C1]

To a stirred solution of Ibrutinib (440 mg, 1 mmol) in 1,4-dioxane:H₂O (3:1 v/v, 12 mL) were added 1,4-diazabicyclo[2.2.2]octane (DABCO) (123.2 mg, 1.1 mmol), phenol (21.8 uL, 0.25 mmol) and paraformaldehyde (600 mg, 20 mmol) at 25° C. The reaction mixture was stirred at room temperature for 3d. After completion of the reaction (as monitored by LC-MS), 1,4 dioxane was concentrated in vacuo and the aqueous layer was extracted with EtOAc (3×30 mL). The combined organic layer was concentrated in vacuo and the crude product was purified by column chromatography over silica gel using EtOAc/Pet. ether as eluent to give pure alcohol (3l) as white solid (286.7 mg, yield=61%)

¹H NMR (400 MHz, DMSO-d₆) δ 1.54 (br. s., 1H), 1.93 (br. s., 1H), 1.99-2.22 (m, 2H), 2.78-2.99 (m, 1H), 3.08-3.22 (m, 1H), 3.25-3.41 (m, 2H), 3.43-3.69 (m, 2H), 4.68 (br. s., 2H), 5.34 (br. s., 1H), 6.98-7.19 (m, 5H), 7.38 (t, J=7.9 Hz, 2H), 7.57 (d, J=7.9 Hz, 2H), 8.17 (s, 1H); ¹³C NMR (100 MHz, DMSO-d₆) δ 25.1, 30.4, 46.0, 47.7, 53.0, 62.9, 98.4, 115.1, 120.0, 120.1, 125.2, 128.2, 131.1, 131.3, 144.8, 144.9, 154.3, 156.6, 156.9, 158.5, 159.0, 170.8.

### (R)-1-(3-(4-amino-3-(4-phenoxyphenyl)-1H-pyrazolo[3,4-d]pyrimidin-1-yl)piperidin-1-yl)-2-(bromomethyl)prop-2-en-1-one (3m)

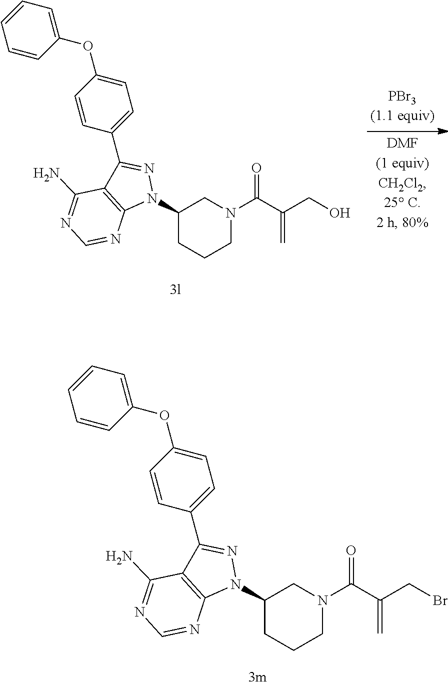 [SMILES: C=C(CO)C(=O)N1CCC[C@@H](n2nc(-c3ccc(Oc4ccccc4)cc3)c3c(N)ncnc32)C1 ; C=C(CBr)C(=O)N1CCC[C@@H](n2nc(-c3ccc(Oc4ccccc4)cc3)c3c(N)ncnc32)C1]

To a stirred solution of alcohol (3l) (235 mg, 0.5 mmol) in CH₂Cl₂ (5 mL) was added PBr₃ (52.5 uL, 0.55 mmol) and DMF (37.5 uL, 0.5 mmol) at 0° C. under N₂ atm. The reaction mixture was allowed to stir at room temperature for 1 h under and quenched with H₂O (5 mL) at 0° C. The aqueous layer was extracted with CH₂Cl₂ (3×8 mL) and concentrated in vacuo. The crude product was purified by column chromatography over silica gel using EtOAc/Pet. ether as eluent to give pure bromo compound as white solid (427 mg, yield=80%).

¹H NMR (500 MHz, CD₃OD) δ 1.85 (br. s., 1H), 2.11 (br. s., 1H), 2.29 (d, J=12.8 Hz, 1H), 2.35-2.49 (m, 1H), 3.43-3.60 (m, 1H), 3.68-3.80 (m, 1H), 3.80-3.90 (m, 1H), 4.05-4.13 (m, 1H), 4.25 (br. s., 1H), 4.29-4.37 (m, 1H), 4.52 (br. s., 2H), 5.02 (br. s., 1H), 5.29-5.40 (m, 1H), 5.68 (br. s., 1H), 7.13 (d, J=8.7 Hz, 2H), 7.16-7.26 (m, 3H), 7.44 (t, J=8.0 Hz, 2H), 7.72 (d, J=8.5 Hz, 2H), 8.43 (br. s., 1H); ¹³C NMR (125 MHz, CD₃OD) δ 25.7, 30.8, 33.5, 43.2, 46.9, 52.4, 54.4, 55.5, 98.4, 119.9, 120.2, 120.3, 120.7, 120.9, 125.3, 125.4, 127.4, 131.3, 131.5, 141.4, 148.6, 148.7, 153.5, 154.8, 157.9, 160.8, 161.8, 162.1, 170.7.

### (R)-1-(3-(4-amino-3-(4-phenoxyphenyl)-1H-pyrazolo[3,4-d]pyrimidin-1-yl)piperidin-1-yl)-2-methylprop-2-en-1-one (3a)

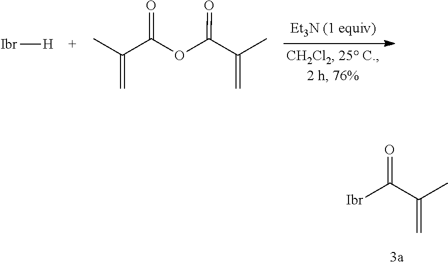

To a stirred solution of Ibr-H (38.7 mg, 0.1 mmol) in anhydrous DCM (0.5 mL) was added Et₃N (13.9 uL, 0.1 mmol) and methacrylic anhydride (15.4 uL, 0.1 mmol) at 25° C. The reaction mixture was stirred at room temperature for 1 h. After completion of the reaction (as monitored by LC-MS), CH₂Cl₂ was concentrated in vacuo. The crude product was purified by High Pressure Liquid Chromatography (HPLC) using water:ACN (0.1% formic acid) solvent gradient to afford white solid 3a in 36.1 mg (79.5% yield).

¹H NMR (500 MHz, CDCl₃) δ 1.66-1.79 (m, 1H), 1.98 (s, 3H), 2.06 (d, J=14 Hz, 1H), 2.22-2.32 (m, 1H), 2.38 (d, J=11 Hz, 1H), 3.24 (br. s., 1H), 3.54 (br. s., 1H), 4.03 (br. s., 1H), 4.69 (br. s., 1H), 4.91 (br. s., 1H), 5.12 (s, 1H), 5.23 (br. s., 1H), 6.34 (br. s., 1H), 7.12 (d, J=8 Hz, 2H), 7.19 (m, J=8 Hz, 2H), 7.23 (t, J=7 Hz, 1H), 7.44 (t, J=8 Hz, 2H), 7.60 (m, J=8 Hz, 2H), 8.28 (s, 1H), 11.55 (br. s., 1H); ¹³C NMR (125 MHz, CDCl₃) δ 20.5, 24.9, 30.2, 45.3, 46.9, 53.4, 97.1, 114.7, 115.9, 117.0, 119.2, 119.9, 124.6, 125.1, 129.7, 130.1, 140.0, 145.8, 147.0, 151.5, 153.6, 155.7, 159.8, 163.4, 163.7, 171.8.

### (R)-1-(3-(4-amino-3-(4-phenoxyphenyl)-1H-pyrazolo[3,4-d]pyrimidin-1-yl)piperidin-1-yl)-2-((diethylamino)methyl)prop-2-en-1-one (3b)

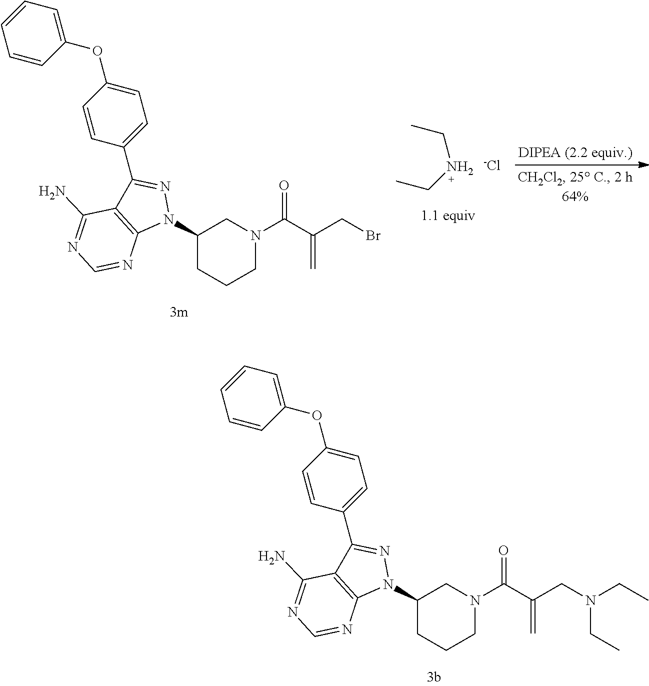

To a stirred solution of diethylamino hydrochloride (5.9 mg, 0.055 mmol) in anhydrous DCM (1 mL) were added DIPEA (19.1 uL, 0.11 mmol) and bromo compound (3m) (26.5 mg, 0.05 mmol) at 25° C. The reaction mixture was allowed to stir at room temperature for one hour. After completion of the reaction (as monitored by LC-MS, CH₂Cl₂ was concentrated in vacuo. The crude product was purified by High-Pressure Liquid Chromatography (HPLC) using water: ACN (0.1% formic acid) solvent gradient to afford white solid of compound 3b (19.4 mg, 64% yield).

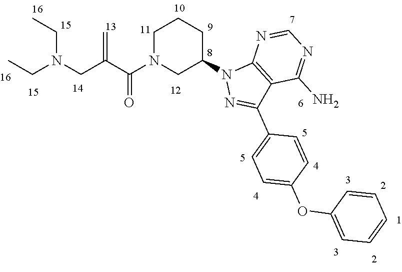 [SMILES: C=C(CN(CC)CC)C(=O)N1CCC[C@@H](n2nc(-c3ccc(Oc4ccccc4)cc3)c3c(N)ncnc32)C1]

¹H NMR (500 MHz, DMSO-d₆) δ 1.22 (t, J=7.08 Hz, 6H¹⁶), 1.71 (br. s., 1H^{10a}), 1.95 (d, J=12.65 Hz, 1H^{9a}), 2.10-2.22 (m, 1H^{10b}), 2.24-2.40 (m, 1H^{9b}), 2.96 (br. s., 1H^{11a}), 3.13 (br. s., 4H¹⁵) 3.29 (br. s., 1H^{11b}), 3.95 (br. s., 2H¹⁴), 4.19 (br. s., 1H^{12a}), 4.32-4.49 (br. s., 1H^{12b}), 4.80-4.91 (m, 1H⁸), 5.80 (br. s., 1H^{13a}), 5.87-6.04 (m, 1H^{13b}), 7.14 (d, J=8.25 Hz, 2H³), 7.17 (d, J=8.53 Hz, 2H⁴), 7.21 (t, J=7.70 Hz, 1H¹), 7.45 (t, J=7.77 Hz, 2H²), 7.67 (d, J=8.39 Hz, 2H⁵), 8.35 (br. s., 1H⁷), 9.18 (br. s., 1H⁶); ¹³C NMR (125 MHz, DMSO-d₆) δ 8.8, 8.9, 9.1, 11.5, 29.7, 29.9, 44.0, 47.0, 47.1, 54.0, 97.7, 115.5, 117.9, 119.4, 119.5, 124.4, 127.8, 127.9, 130.6, 130.6, 144.5, 153.9, 156.7, 157.8, 158.5, 158.8, 167.9.

### (R)-1-(3-(4-amino-3-(4-phenoxyphenyl)-1H-pyrazolo[3,4-d]pyrimidin-1-yl)piperidin-1-yl)-2-(piperidin-1-ylmethyl)prop-2-en-1-one (3c)

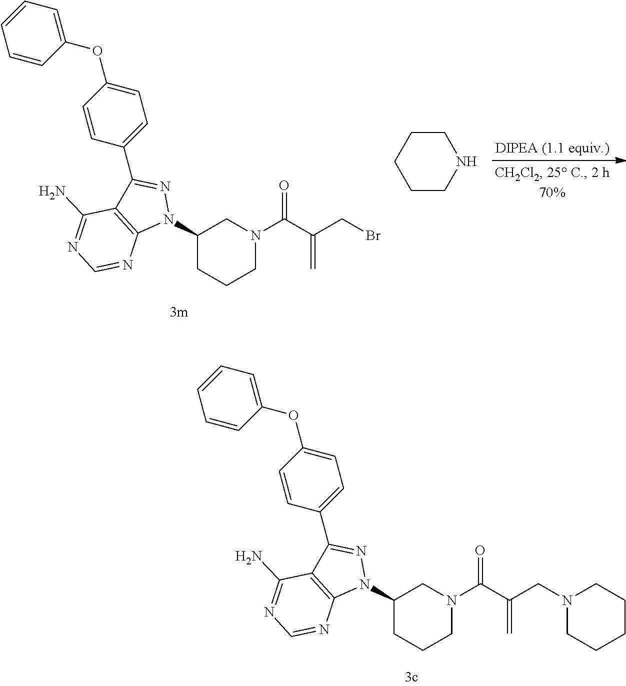 [SMILES: C=C(CBr)C(=O)N1CCC[C@@H](n2nc(-c3ccc(Oc4ccccc4)cc3)c3c(N)ncnc32)C1 ; C1CCNCC1 ; C=C(CN1CCCCC1)C(=O)N1CCC[C@@H](n2nc(-c3ccc(Oc4ccccc4)cc3)c3c(N)ncnc32)C1]

To a stirred solution of bromo compound (3m) (26.5 mg, 0.05 mmol) in anhydrous DCM (1 mL) were added piperidine (5.43 uL, 0.055 mmol) and N,N-Diisopropylethylamine (DIPEA) (9.8 uL, 0.055 mmol) at 25° C. The reaction mixture was allowed to stir at room temperature for one hour. After completion of the reaction (as monitored by LC-MS), CH₂Cl₂ was concentrated in vacuo. The crude product was purified by High-Pressure Liquid Chromatography (HPLC) using water: ACN (0.1% formic acid) solvent gradient to afford white solid (18.8 mg, 70% yield).

¹H NMR (500 MHz, CD₃OD): δ 1.71-1.91 (m, 4H), 1.97 (m, 2H), 2.11 (br. s., 1H), 2.25-2.34 (m, 1H), 2.43 (br. s., 1H), 2.82-3.00 (m, 2H), 3.44-3.65 (m, 3H), 3.73 (br. s., 1H), 3.85 (br. s., 1H), 3.88-4.07 (m, 2H), 4.32 (br. s., 1H), 4.49 (br. s., 1H), 5.05 (br. s., 1H), 5.85 (s, 1H), 5.94 (br. s., 1H), 7.13 (d, J=7.8 Hz, 2H), 7.19 (d, J=8.7 Hz, 2H), 7.21-7.25 (m, 1H), 7.44 (t, J=8.0 Hz, 2H), 7.71 (d, J=8.5 Hz, 2H), 8.42 (s, 1H); ¹³C NMR (125 MHz, CD₃OD) δ 22.8, 24.3, 25.5, 30.8, 34.2, 43.3, 47.0, 52.3, 54.2, 54.6, 60.7, 98.5, 120.2, 120.9, 125.5, 127.5, 128.9, 131.3, 131.5, 133.3, 148.3, 153.7, 155.3, 157.8, 160.8, 162.6, 162.9, 170.0.

### (R)-1-(3-(4-amino-3-(4-phenoxyphenyl)-1H-pyrazolo[3,4-d]pyrimidin-1-yl)piperidin-1-yl)-2-((methyl(phenyl)amino)methyl)prop-2-en-1-one (3d)

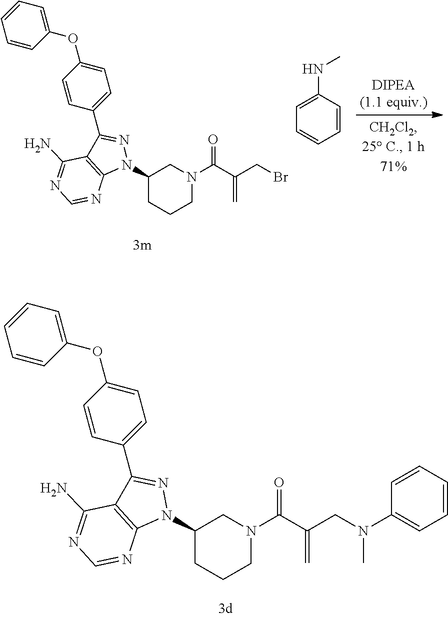

To a stirred solution of bromo compound (3m) (26.5 mg, 0.05 mmol) in anhydrous DCM (1 mL) were added N-methyl aniline (5.95 uL, 0.055 mmol) and DIPEA (9.8 uL, 0.055 mmol) at 25° C. The reaction mixture was allowed to stir at room temperature for one hour. After completion of the reaction (as monitored by LC-MS), CH₂Cl₂ was concentrated in vacuo. The crude product was purified by High-Pressure Liquid Chromatography (HPLC) using water: ACN (0.1% formic acid) solvent gradient to afford white solid (19.8 mg, 71% yield) of compound 3d.

¹H NMR (500 MHz, CD₃OD) δ 1.49 (br. s., 1H), 1.98 (br. s., 1H), 2.19 (br. s., 1H), 2.24 (br. s., 1H), 2.31 (br. s., 1H), 3.02 (br. s., 3H), 3.66 (dd, J=13.1, 9.4 Hz, 1H), 3.82 (br. s., 1H), 4.08-4.18 (m, 1H), 4.19-4.29 (m, 1H), 4.39 (br. s., 1H), 5.24-5.34 (m, 1H), 5.41 (br. s., 1H), 6.79 (br. s., 1H), 6.83-6.93 (m, 1H), 7.10 (d, J=7.3 Hz, 2H), 7.14-7.28 (m, 4H), 7.42 (t, J=7.9 Hz, 2H), 7.69 (d, J=8.3 Hz, 2H), 8.37-8.46 (m, 1H); ¹³C NMR (125 MHz, CD₃OD) δ 25.4, 30.8, 40.1, 46.9, 52.3, 54.4, 57.8, 98.5, 114.8, 120.4, 121.0, 124.1, 125.6, 127.4, 130.6, 131.6, 132.6, 141.7, 148.3, 148.6, 149.8, 153.5, 158.0, 161.0, 172.2.

### (R)-1-(3-(4-amino-3-(4-phenoxyphenyl)-1H-pyrazolo[3,4-d]pyrimidin-1-yl)piperidin-1-yl)-2-((benzylamino)methyl)prop-2-en-1-one (3e)

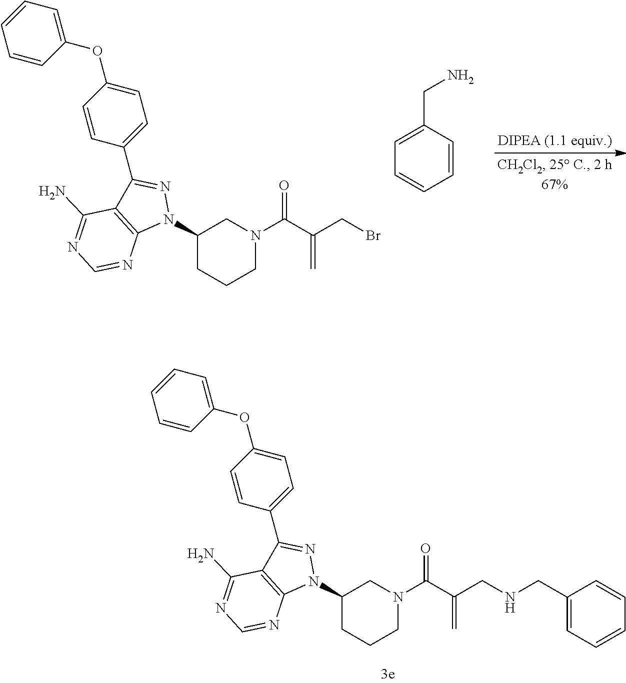 [SMILES: C=C(CBr)C(=O)N1CCC[C@@H](n2nc(-c3ccc(Oc4ccccc4)cc3)c3c(N)ncnc32)C1 ; NCc1ccccc1 ; C=C(CNCc1ccccc1)C(=O)N1CCC[C@@H](n2nc(-c3ccc(Oc4ccccc4)cc3)c3c(N)ncnc32)C1]

To a stirred solution of bromo compound (3m) (26.5 mg, 0.05 mmol) in anhydrous DCM (1 mL) were added benzyl amine (6.0 uL, 0.055 mmol) and DIPEA (9.8 uL, 0.055 mmol) at 25° C. The reaction mixture was allowed to stir at room temperature for one hour. After completion of the reaction (as monitored by LC-MS), CH₂Cl₂ was concentrated in vacuo. The crude product was purified by High-Pressure Liquid Chromatography (HPLC) using water: ACN (0.1% formic acid) solvent gradient to afford white solid (18.7 mg, 67% yield) of compound 3e.

¹H NMR (500 MHz, CD₃OD): δ 1.77 (br. s., 1H), 2.08 (br. s., 1H), 2.27 (m, 1H), 2.39 (br. s., 1H), 3.44-3.68 (br. s., 1H), 3.76-3.87 (m, 2H), 4.20 (br. s., 2H), 4.32 (br. s., 1H), 4.48 (br. s., 1H), 4.99 (br. s., 1H), 5.78 (br. s., 1H), 5.86 (br. s., 1H), 7.09 (d, J=7.8 Hz, 2H), 7.14-7.22 (m, 3H), 7.39 (s, 2H), 7.45-7.54 (m, 5H), 7.69 (d, J=8.5 Hz, 2H), 8.35 (br. s., 1H); ¹³C NMR (125 MHz, CD₃OD) δ 25.6, 30.9, 43.3, 47.0, 50.8, 52.0, 54.0, 98.8, 119.2, 120.1, 120.9, 125.4, 126.5, 127.9, 130.6, 131.0, 131.2, 131.3, 131.4, 132.4, 134.6, 140.6, 147.7, 151.4, 154.2, 157.9, 160.7, 162.8, 170.2.

### (R)-1-(2-(3-(4-amino-3-(4-phenoxyphenyl)-1H-pyrazolo[3,4-d]pyrimidin-1-yl)piperidine-1-carbonyl)allyl)-4-(dimethylamino)pyridin-1-ium (3f)

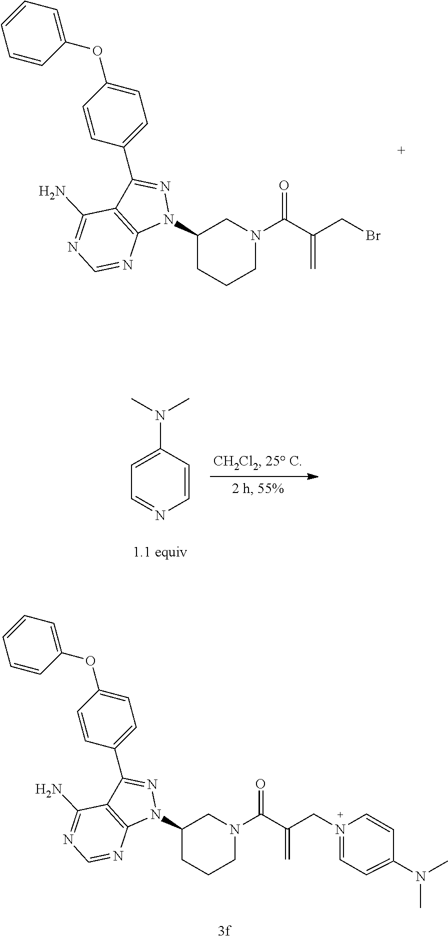

To a stirred solution of bromo compound (3m) (26.5 mg, 0.05 mmol) in anhydrous DCM (1 mL) were added N,N-dimethylaminopyridine (6.7 mg, 0.055 mmol) at 25° C. The reaction mixture was allowed to stir at room temperature for one hour. After completion of the reaction (as monitored by LC-MS), CH₂Cl₂ was concentrated in vacuo. The crude product was purified by High-Pressure Liquid Chromatography (HPLC) using water: ACN (0.1% formic acid) solvent gradient to afford white solid (15.8 mg, 55% yield) of compound 3f.

¹H NMR (500 MHz, CD₃OD): δ 1.61 (m, 1H), 2.03 (br. s., 1H), 2.22 (m, 1H), 2.34 (br. s., 1H), 3.26 (s, 6H), 3.40 (br. s., 1H), 3.69 (br. s., 1H), 3.93 (br. s., 1H), 4.34 (br. s., 2H), 4.98 (br. s., 2H), 5.61 (br. s., 2H), 7.01 (d, J=7.6 Hz, 3H), 7.09 (m, J=8.1 Hz, 2H), 7.13-7.24 (m, 3H), 7.41 (t, J=7.8 Hz, 2H), 7.68 (m, J=8.5 Hz, 2H), 8.06-8.21 (m, 2H), 8.39 (s, 1H); ¹³C NMR (125 MHz, CD₃OD) δ 24.0, 29.3, 32.7, 39.0, 41.6, 45.4, 52.5, 59.1, 97.1, 107.6, 115.4, 117.7, 118.6, 119.2, 120.8, 123.9, 126.3, 129.7, 137.7, 141.9, 156.4, 156.7, 159.1, 160.8, 161.1, 161.3, 161.6, 167.8.

### (R)-2-(3-(4-amino-3-(4-phenoxyphenyl)-1H-pyrazolo[3,4-d]pyrimidin-1-yl)piperidine-1-carbonyl)allyl acetate (3g)

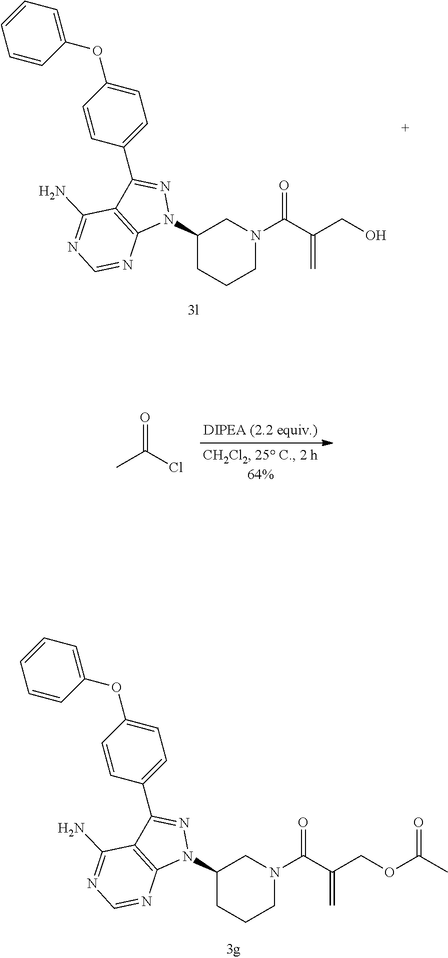

To a stirred solution of alcohol (3l) (22 mg, 0.05 mmol) in anhydrous DCM (0.5 mL) were added acetyl chloride (4.25 uL, 0.06 mmol) and DIPEA (10.6 uL, 0.06 mmol) at 25° C. The reaction mixture was allowed to stir at room temperature for 1 h. After completion of the reaction (as monitored by LC-MS), CH₂Cl₂ was concentrated in vacuo. The crude product was purified by High-Pressure Liquid Chromatography (HPLC) using water:ACN (0.1% formic acid) solvent gradient to afford white solid 3g (16.3 mg, 64% yield).

¹H NMR (400 MHz, CD₃OD): δ 1.85 (d, J=10.3 Hz, 1H), 2.17 (br. s., 4H), 2.27-2.42 (m, 1H), 2.50 (d, J=9.2 Hz, 1H), 3.58 (br. s., 1H), 3.84 (br. s., 1H), 4.08 (br. s., 1H), 4.40 (br. s., 1H), 4.49 (br. s., 1H), 4.84 (br. s., 2H), 5.08 (br. s., 1H), 5.48 (br. s., 1H), 5.59 (br. s., 1H), 5.65 (br. s., 1H), 7.15-7.34 (m, 5H), 7.44-7.59 (m, 2H), 7.79 (d, J=8.6 Hz, 2H), 8.50 (s, 1H); ¹³C NMR (100 MHz, CD₃OD) δ 20.8, 24.0, 30.8, 43.0, 46.8, 54.2, 65.9, 98.5, 119.1, 119.8, 120.2, 120.4, 120.9, 127.4, 129.9, 131.0, 131.2, 131.3, 131.5, 140.9, 148.4, 153.6, 155.8, 157.9, 158.5, 159.7, 160.8, 170.9, 172.4.

### (R)-2-(3-(4-amino-3-(4-phenoxyphenyl)-1H-pyrazolo[3,4-d]pyrimidin-1-yl)piperidine-1-carbonyl)allyl acetate (3h)

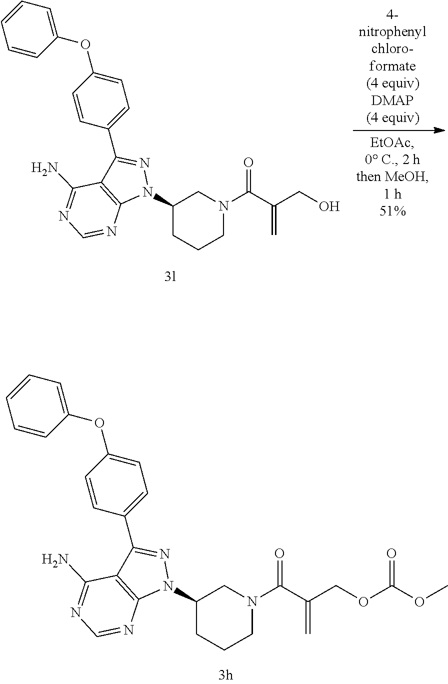 [SMILES: C=C(CO)C(=O)N1CCC[C@@H](n2nc(-c3ccc(Oc4ccccc4)cc3)c3c(N)ncnc32)C1 ; C=C(COC(=O)OC)C(=O)N1CCC[C@@H](n2nc(-c3ccc(Oc4ccccc4)cc3)c3c(N)ncnc32)C1]

To a stirred solution of alcohol (3l) (23.5 mg, 0.05 mmol) in ethyl acetate (0.5 mL) were added 4-nitrophenyl chloroformate (40.8 mg, 0.2 mmol) and 4-Dimethylaminopyridine (24.4 mg, 0.2 mmol) at 0° C. The reaction mixture was allowed to stir at room temperature for 2 h. After completion of the reaction (as monitored by LC-MS, the reaction mixture is quenched with 0.1 N HCl (2 mL) and the aqueous layer was extracted with EtOAc (3×3 mL). The organic layer was concentrated in vacuo and dissolved in MeOH (0.5 mL). The reaction is further allowed to stir for 1 hour at room temperature. After completion of the reaction, methanol was concentrated and the crude product was purified by High Pressure Liquid Chromatography (HPLC) using water:ACN (0.1% formic acid) solvent gradient to afford white solid 3h in (13.4 mg, 51% yield).

¹H NMR (400 MHz, CD₃OD): δ 1.80-1.91 (m, 1H), 2.15-2.23 (m, 1H), 2.33-2.40 (m, 1H), 2.50 (br. s., 1H), 3.56 (br. s., 1H), 3.87 (br. s., 3H), 3.99-4.17 (m, 1H), 4.46-4.60 (m, 1H), 4.87 (d, J=12.1 Hz, 2H), 5.04-5.15 (m, 1H), 5.51 (br. s., 1H), 5.68 (br. s., 1H), 7.12-7.37 (m, 5H), 7.48-7.56 (m, 2H), 7.79 (d, J=8.6 Hz, 2H), 8.50 (s, 1H); ¹³C NMR (100 MHz, CD₃OD) δ 25.4, 28.9, 30.8, 46.8, 54.2, 55.7, 69.2, 119.6, 120.2, 120.8, 125.4, 127.5, 130.2, 131.3, 131.5, 133.2, 140.5, 148.4, 149.0, 157.0, 157.9, 160.8, 170.7.

### (R)-1-(3-(4-amino-3-(4-phenoxyphenyl)-1H-pyrazolo[3,4-d]pyrimidin-1-yl)piperidin-1-yl)-2-(phenoxymethyl)prop-2-en-1-one (3i)

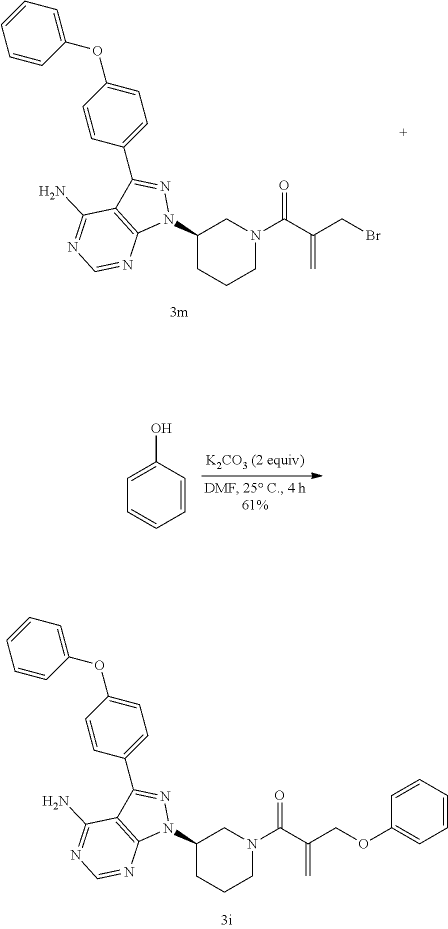 [SMILES: C=C(CBr)C(=O)N1CCC[C@@H](n2nc(-c3ccc(Oc4ccccc4)cc3)c3c(N)ncnc32)C1 ; Oc1ccccc1 ; C=C(COc1ccccc1)C(=O)N1CCC[C@@H](n2nc(-c3ccc(Oc4ccccc4)cc3)c3c(N)ncnc32)C1]

To a stirred solution of bromo compound (3m) (26.5 mg, 0.05 mmol) in dry DMF (1 mL), phenol (4.7 uL, 0.055 mmol) and K₂CO₃ (15.2 mg, 0.11 mmol), were added at 25° C. under an N₂ atmosphere. The reaction mixture was allowed to stir at room temperature for 4 h. After completion (as monitored by LC-MS), the reaction mixture was quenched with H₂O (2 mL). The aqueous layer was extracted with EtOAc (3×3 mL). The organic layer was washed with brine and concentrated in vacuo. The crude product was purified by High-Pressure Liquid Chromatography (HPLC) using water:ACN (0.1% formic acid) solvent gradient to afford white solid (yield 16.6 mg, 61%).

¹H NMR (400 MHz, CD₃OD) δ 1.84 (br. s., 1H), 2.33-2.41 (m, 1H), 2.46 (d, J=11.4 Hz, 1H), 3.00 (br. s., 1H), 3.25-3.48 (m, 1H), 3.68 (br. s., 1H), 3.86 (br. s., 1H), 4.66-4.84 (m, 2H), 4.88 (br. s., 1H), 4.99-5.11 (m, 1H), 5.48 (br. s., 1H), 5.72 (br. s., 1H), 6.47 (br. s., 1H), 6.96-7.12 (m, 2H), 7.21 (m, J=7.7 Hz, 2H), 7.27 (d, J=8.1 Hz, 1H), 7.32-7.43 (m, 3H), 7.46-7.59 (m, 2H), 7.67 (m, J=8.4 Hz, 2H), 8.40 (br. s., 1H), 10.56 (br. s., 1H); ¹³C NMR (100 MHz, CD₃OD): δ 24.6, 30.2, 45.6, 47.3, 53.3, 68.8, 96.9, 114.6, 118.1, 119.2, 120.0, 121.4, 124.7, 129.5, 129.7, 130.2, 139.7, 145.0, 147.2, 151.4, 153.0, 155.5, 158.1, 160.0, 169.7.

### Example 3

### Reactivity and of α-Methacrylamides Compounds

To investigate the reactivity and leaving ability of α-substituted methacrylamides, A set of nine model compounds of various α-substituted N-benzyl-methacrylamides (1b-1j; Table 1) have been synthesized from the corresponding N-benzyl-2-(bromomethyl) acrylamide (Example 2), as well as the unsubstituted acrylamide (BnA) and methacrylamide (1a). These electrophiles were reacted with reduced glutathione (GSH), as a model thiol and monitored the reaction over time via liquid chromatography/mass spectrometry (LC/MS). As an example, analysis of the reaction of 1g (which has coumarin as a substituent) after 0.5 h and 48 h (FIG. 4A), clearly indicates the formation of a substitution product, the formation of 7-hydroxy coumarin, and decrease of starting material. The product formation was quantified via LC/MS of all model compounds and assessed the reaction rates (5 mM GSH; 100 μM acrylamide; 37° C.; Table 1, FIG. 4A).

The substituted methacrylamides, were more reactive than the unsubstituted acrylamide. There was a clear correlation between the pKₐ of the leaving group (pK_{b} in the case of amines)²³⁻²⁵ and the t_{1/2} of the model compounds reaction with GSH (FIG. 4B).

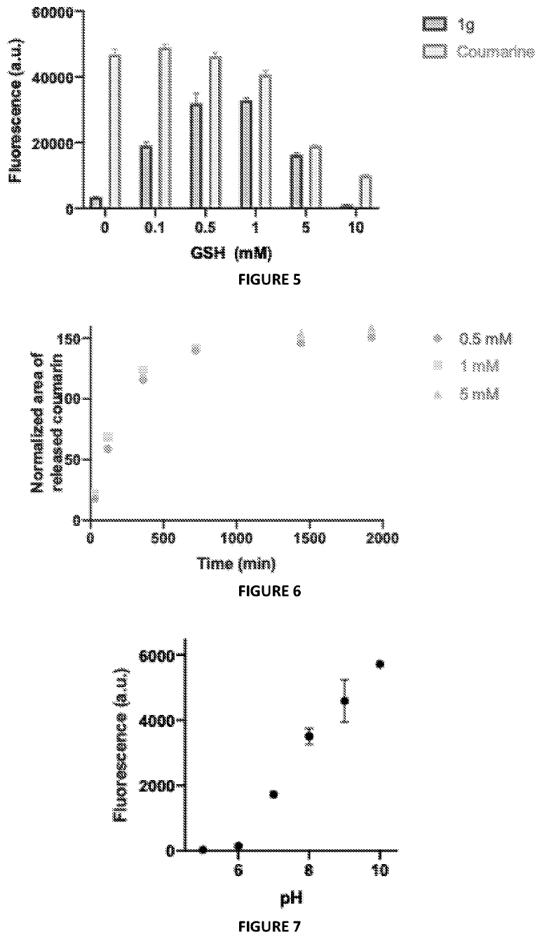

Compound 1g, which in and of itself is not fluorescent, releases coumarin as the leaving group upon reaction with GSH, therefore allowing us to follow the reaction by a turn-on fluorescent readout. Indeed, fluorescence monitoring of the reaction (5 mM GSH, 100 μM 1g, pH 8; FIG. 4C) showed a similar rate to that obtained by LC/MS. To understand the effect of GSH concentration on the fluorescence of 1g, the fluorescence readout was followed at various concentrations of GSH (FIG. 4C). The fluorescence increased as a function of GSH concentration and reaches its maximum at 1 mM of GSH. Further increase in GSH concentration decreased the fluorescence likely due to acidification of the buffer. The decrease in fluorescence of 7-hydroxycoumarin as a control at the high GSH concentrations was observed (FIG. 5). Furthermore, when the reaction of 1g was monitored with different concentrations of GSH (0.5, 1, and 5 mM) by LC/MS, a similar rate for the release of 7-hydroxy coumarin was observed in all three cases (FIG. 6), indicating that the decrease in fluorescence signal was primarily due to the reduction in intrinsic 7-hydroxycoumarin fluorescence at low pH. Indeed, fixing the concentration of the reactants (5 mM GSH; 100 μM 1g) and varying the pH showed a linear effect of the fluorescent signal as a function of pH (FIG. 7), with increasing reaction rates (FIG. 4D) and maximal fluorescence at higher pH values.

TABLE 1
Various hetero-substitutions of α-methacrylamides span 2.5 orders of
magnitude in reactivity towards GSH.

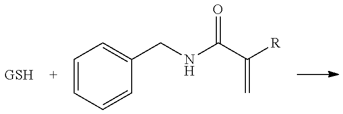

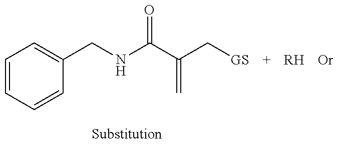

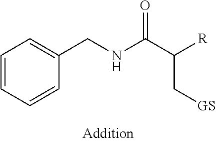

| Compound | R^{a} | GSH t_{1/2} (hours)^{b} | Substitution/Addition^{b} |
|---|---|---|---|
| BnA | H | >100 | Addition |
| 1a | CH₃ | >100 | Neither |
| 1b | 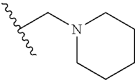 [SMILES: *CN1CCCCC1] | 0.3 | Substitution/Addition ^{~}0%/60% |
| 1c | 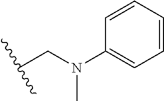 [SMILES: *CN(C)c1ccccc1] | 66 | Substitution |
| 1d | 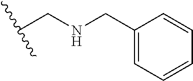 [SMILES: *CNCc1ccccc1] | 0.7 | Substitution/Addition ^{~}40%/60% |
| 1e | 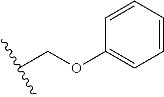 [SMILES: *COc1ccccc1] | 9.9 | Substitution |
| 1f | 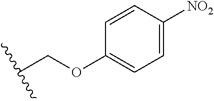 [SMILES: *COc1ccc([N+](=O)[O-])cc1] | 2.6 | Substitution |
| 1g | 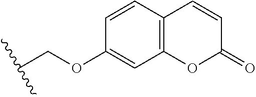 [SMILES: *COc1ccc2ccc(=O)oc2c1] | 3.9 | Substitution |
| 1h | 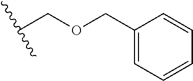 [SMILES: *COCc1ccccc1] | >100 | Addition |
| 1i | 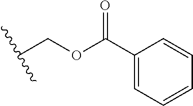 [SMILES: *COC(=O)c1ccccc1] | 1.6 | Substitution |
| 1j | 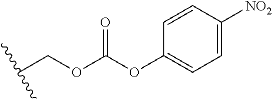 [SMILES: *COC(=O)Oc1ccc([N+](=O)[O-])cc1] | 0.9 | Substitution |

The reaction of substituted α-methacrylamides with GSH can result in either a substitution or addition product.
^{a}Model substituted α-methacrylamides
^{b}Reactivity towards GSH (t_{1/2}) and reaction type were assessed via LC/MS (FIG. 4A).

### Example 4

### Proteomic Reactivity of Substituted α-Methacrylamides

To assess the proteomic reactivity of this new electrophile three model alkynes (Example 2) bearing an α-methacrylamide substituted with either coumarin, N-methyl aniline, or benzoic acid were synthesized (2a-c; FIG. 8A). The coumarin derivatized alkyne 2a shows similar reactivity to 1g in a GSH-triggered fluorescence assay (FIG. 9). Mino cells were treated for two hours with either DMSO, IA-alkyne, or 2a-c. The cells were lysed, labeled the alkynes via copper-catalyzed “click chemistry” with TAMRA (Tetramethylrhodamine)-azide, and imaged the adducts via in-gel fluorescence (FIG. 8B). Compound 2a showed slightly higher reactivity than 2b, similar to the GSH results with 1g and 1c. 2c however, seemed completely inactive in this experiment, despite 1i showing higher reactivity in the GSH experiment. This may be the result of cellular esterases that hydrolyze the ester, leaving an unreactive substituted acrylamide²⁶. All of the acrylamides were markedly less reactive than IA-alkyne.

### Example 5

### Irreversible Kinase Inhibitors

To assess this chemistry in the context of irreversible covalent inhibitors Ibrutinib was chosen as a model compound. Ibrutinib is an irreversible inhibitor of Bruton's tyrosine kinase (BTK) and is FDA approved for several B cell oncogenic malignancies.²⁷ Starting from the parent Ibrutinib, the Morita-Baylis-Hillmann reaction was used to functionalize the acrylamide and have synthesized various Ibrutinib based meth-acrylamide derivatives with different leaving groups including phenols, acids, carbonates, amines, and quaternary ammonium salts (3a-3j; Example 2, FIG. 10A). All of these compounds were able to show the covalent binding of the recombinant BTK kinase domain as assessed by intact protein mass spectrometry (FIG. 10B; Table 2).

TABLE 2
Properites of α-substituted derivatives of ibrutinib.

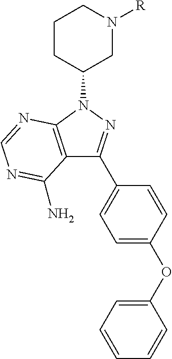

| | | | BTK | | | GSH |
|---|---|---|---|---|---|---|
| | | BHK t_{1/2} | Substitution/ | | GSH t_{1/2} | Substitution/ |
| Compound | R^{a} | (min)^{b} | Addition | IC₅₀ (nM) | (hour)^{b} | Addition |
| Ibrutinib | 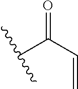 [SMILES: *C(=O)C=C] | <5 | | 0.2 | 2 | |
| 3a | 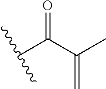 [SMILES: *C(=O)C(=C)C] | >420 | Substitution | 12.2 | No reaction | Substitution |
| 3b | 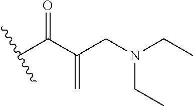 [SMILES: *C(=O)C(=C)CN(CC)CC] | <5 | Substitution/ Addition ^{~}40%/60% | 0.1 | 2 | Substitution |
| 3c | 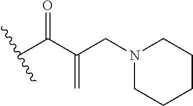 [SMILES: *C(=O)C(=C)CN1CCCCC1] | <5 | Addition | 0.1 | 7 | Substitution |
| 3d | 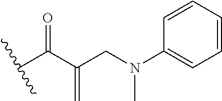 [SMILES: *C(=O)C(=C)CN(C)c1ccccc1] | 47 | Substitution | 5.6 | >100 | Substitution |
| 3e | 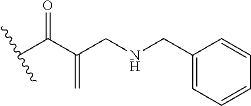 [SMILES: *C(=O)C(=C)CNCc1ccccc1] | <5 | Addition | 0.1 | >100 | Substitution |
| 3f | 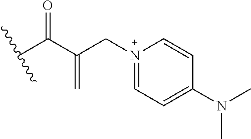 | <5 | Substitution/ Addition ^{~}30%/70% | 0.1 | 7 | Substitution |
| 3g | 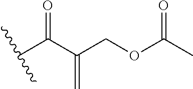 [SMILES: *C(=O)C(=C)COC(C)=O] | <5 | Substitution | 0.1 | 59 | Substitution |
| 3h | 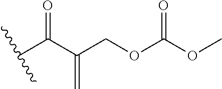 [SMILES: *C(=O)C(=C)COC(=O)OC] | <5 | Substitution | 0.1 | 12 | Substitution |
| 3i | 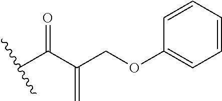 [SMILES: *C(=O)C(=C)COc1ccccc1] | 8 | Substitution | 0.2 | 10 | Substitution |

^{a}Substituted α-methacrylamides analogs of Ibrutinib.
^{b}Reactivity towards GSH (t_{1/2}) and reaction type were assessed via LC/MS.

Similar to the model compounds, phenols, acids, carbonates, and aniline derivatives (3j, 3g, 3h, and 3d) showed 100% labeling through the substitution mechanism within 30 minutes. Basic amine derivatives such as 3b and 3f showed mixed binding with about 35% binding by substitution and 65% binding through Michael addition after two hours of incubation. Finally, 3c and 3e are labeled exclusively through addition with no substitution product.

BTK labeling rates were examined, which now may depend both on tuned intrinsic thiol reactivity, as well as by potentially modified reversible protein recognition. Most compounds were comparable to Ibrutinib, less than two-fold higher or lower, regardless of the reaction mechanism observed (FIG. 10B). Two of the compounds that labeled BTK the slowest, 3i and 3d, correspond to two of the slowest model compounds 1e and 1e respectively. Once again, amine modifications that react solely through the addition mechanism, such as 3e and 3c are amongst the fastest reacting, with t_{1/2} 50% faster than Ibrutinib (FIG. 10B).

To understand the potential of these compounds as inhibitors, an in vitro kinase activity assays were conducted for all the Ibrutinib derivatives against BTK. The IC₅₀s of these compounds (FIG. 10C) closely mirrored the BTK kinetic labeling experiments. With some of the ester, carbonate, and basic amine substituted inhibitors such as 3c, 3g, 3h, and 3e showing IC₅₀ in the <100 μM range, better than Ibrutinib (IC₅₀=288 μM). Other compounds inhibited BTK with IC₅₀ in the 100 μM-12 nM range (FIG. 10C).

Further, a GSH based reactivity assay for all the Ibrutinib derivatives has been conducted (FIG. 10D).

To assess the compatibility of this chemistry with cellular conditions an evaluation of B cell receptor signaling inhibition in primary mouse B cells by Ibrutinib as well as four of our new inhibitors was made. B cells were incubated (24 h; 37° C.) with the inhibitors at various concentrations, treated with anti-IgM, and activation was assessed by flow cytometry detection of CD86 expression. All four inhibitors with substituted methacrylamides (3c, 3e, 3g, and 3h) showed similar activity to Ibrutinib, indicating both cellular engagement as well as stability to cellular conditions.

### Example 6

### Covalent Ligand Directed Release (CoLDR) Chemistry for Functionalization of Irreversible Inhibitors

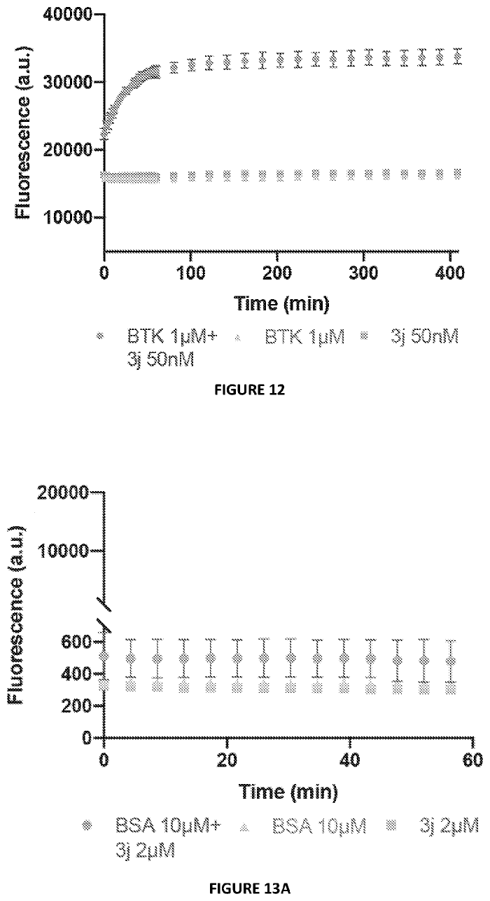

The fact that a specific leaving group is released as a function of selective binding of a target protein can be used to functionalize irreversible inhibitors, for example as turn-on fluorescent probes. To assess the applicability and generality of this approach three therapeutic targets were chosen for which acrylamide inhibitors are available: BTK, EGFR, and K-RAS^{G12C} as model systems. Initially, 3j (FIG. 11A) was treated with BTK, measured the released coumarin fluorescence, and validated the labeling via LC/MS (FIG. 11D, 11G). The fluorescence intensity of 3j at 435 nm increased 30 fold upon the addition of BTK within a few seconds, reaching saturation within 10 minutes. To validate that the increase in fluorescence is due to the release of coumarin after binding to BTK, repetition of the experiment with BTK that was pre-incubated with a non-covalent analog of Ibrutinib (Ibr-H) was preformed. In this experiment, the increase in fluorescence was significantly slower due to the gradual displacement of the Ibr-H by 3j. The rate of the reaction was lowered by using 20:1 equivalents of protein:probe (FIG. 12). The fluorescence was increased corresponding to the interaction with BTK, since incubating 3j with BSA did not result in increased fluorescence (FIG. 13A). The release can also be inhibited by pre-incubation of BTK with Iodoacetamide alkyne (IAA; FIG. 13B).

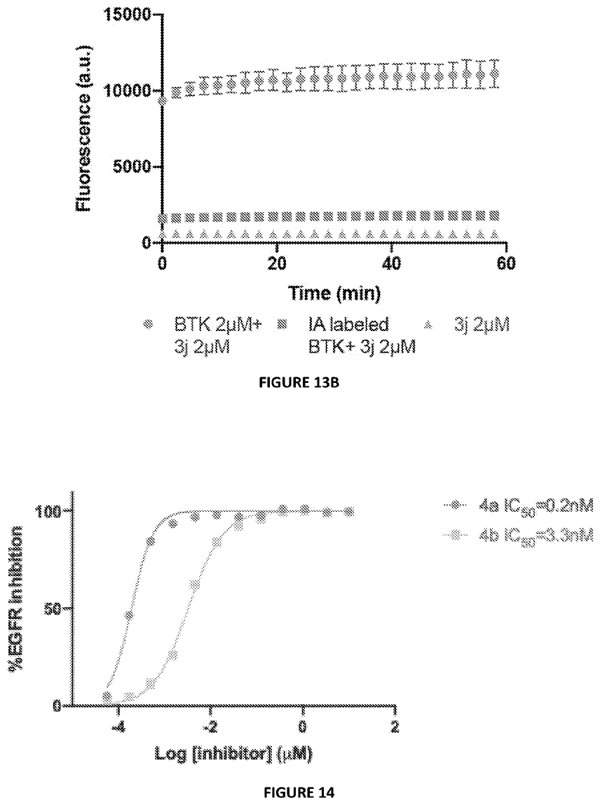

Similarly, 4b (FIG. 11B, Example 1; afatinib derivative functionalized with coumarin) and 5a (FIG. 11C, Example 1; AMG-510 derivative functionalized with coumarin) were treated with EGFR and K-RAS^{G}12c respectively and measured the released coumarin fluorescence (FIG. 11E, 11F). A significant increase in fluorescence intensity was observed in both cases with slower kinetics compared to BTK. LC/MS measurements at the end of the fluorescence measurements showed a shift in the molecular weight of the protein correlating to the size of the labeled compound without the released coumarin (FIG. 11G-11I). In an EGFR kinase activity assay, while 4b was slightly less potent than the unsubstituted 4a (FIG. 14) it still showed an impressive IC₅₀=3.3 nM against EGFR.

Recently, adamantylidene-dioxetane based chemiluminescent turn-on probes for the sensing and imaging of enzymes, reactive oxygen species, and other analytes were reported²⁸⁻³² These probes, upon activation by analytes, release a phenolate-dioxetane intermediate which subsequently decomposes with the emission of a photon in the visible spectrum (FIG. 3). Indeed, these probes showed high sensitivity and signal to background ratios. Accordingly, an Ibrutinib derived chemiluminescent probe for activation by BTK (FIG. 3) was synthesized. The chemiluminescence light emission profile of probe 3k (FIG. 15A, Example 1) upon activation with BTK (2 μM) was measured in the absence and presence of BTK (FIG. 15B). The kinetic profile in the presence of BTK was typical of a chemiluminescent probe with an initial signal was increased to a maximum within 20 minutes, followed by a slow decrease. BTK significantly enhanced chemiluminescence of 3k about 90-fold higher than the total photon counts emitted by probe 3k in the absence of BTK. Pre-incubation of BTK with Ibr-H showed a significant decrease in the luminescence detected, indicating that this probe can be used to measure BTK binding.

The emission profile of probe 3k (FIG. 15A) was measured in the absence and presence of BTK (2 μM; FIG. 15B). The kinetic profile in the presence of BTK was typical of a chemiluminescent probe with an initial signal increase to a maximum within 20 minutes, followed by a slow decrease. BTK significantly enhanced the chemiluminescence of 3k to 90-fold higher than the total photon counts emitted by probe 3k in the absence of BTK. Pre-incubation of BTK with Ibr-H showed a significant decrease in the luminescence detected, indicating that this probe can be used to measure BTK binding.

To demonstrate the possible usage of such compounds, a high throughput screen of ˜4,000 bio-active compounds was conducted. Overall 488 compounds (13%) showed some inhibition of BTK of which 216 (6%) inhibited at least 70% of the signal. 121 out of the 216 strong hit compounds are known kinase inhibitors and 11 out of the 12 known BTK inhibitors in the library were identified as strong hits (FIG. 15C).

### Example 7

### CoLDR Chemistry for the Release of Active Cytotoxic Drugs

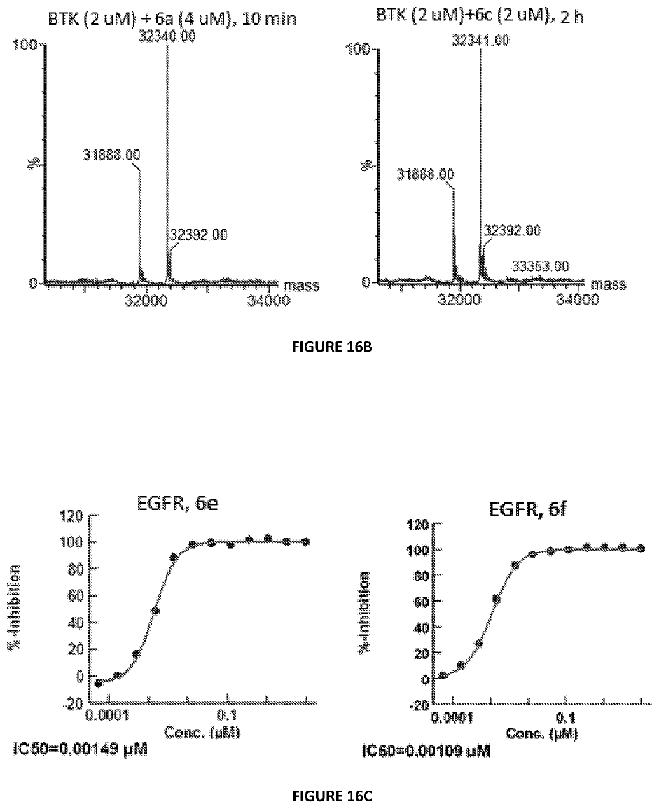

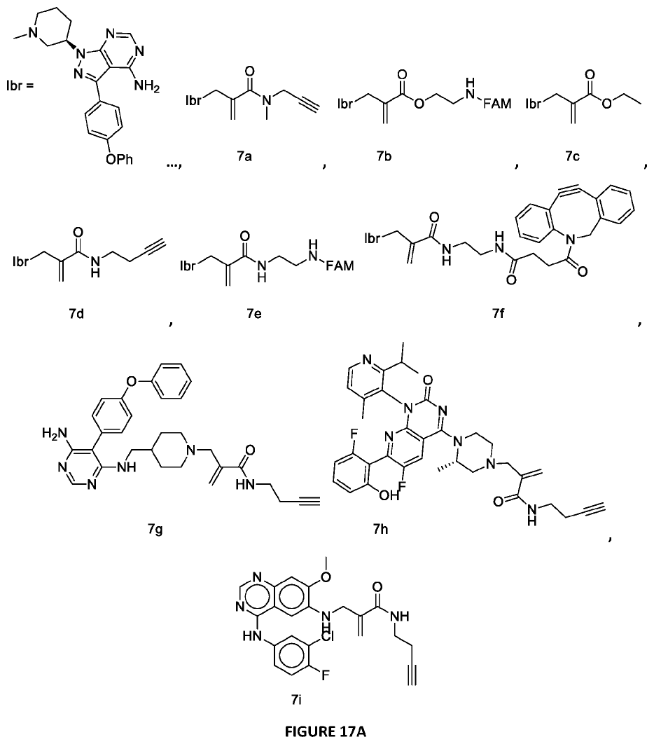

After fluorescent and chemiluminescent turn-on using CoLDR chemistry, the release a toxin turn-on was studied. Some drugs and chemotherapeutic agents are inactive when substituted at particular positions. Examples include amine substitutions of doxorubicin and hydroxy substitutions of camptothecin. In these cases if a chemotherapy drug (in its inactive form) is attached through CoLDR chemistry to the protein binding ligand in such a way that it will be released after the covalent reaction with the protein, it will become toxic only upon release. Ibrutinib attached to camptothecin (6b and 6c) and doxorubicin (6a) were synthesized through a meth-acrylamide for CoLDR chemistry (FIG. 16).

These compounds were treated with reduced glutathione (GSH) to check their releasing ability. Certainly, all three compounds released the corresponding toxin which is identified by LC-MS analysis. Further, when incubating these compounds with the BTK kinase domain, the release of toxins were identified by finding the m/z corresponding to BTK+compound with the release of linker and toxin (FIG. 16B).

To make this releasing chemistry more general, the afatinib derivatives of chemotherapeutic agents were synthesized (6d, 6e, 6f, 6g, 6h). The LC-MS analysis of these compounds in reaction with GSH shows that they can be used for CoLDR chemistry. Further, the in vitro kinase assay of these compounds against EGFR shows that they exhibit nano molar (1 nM) potency FIG. 16C). This indicates modification of afatinib with the chemotherapeutic motifs doesn't interfere with EGFR binding.

### Example 8

### Reverse CoLDR Chemistry for Specific Labelling of Functional Tags on Proteins

Site-selective labeling of proteins plays an important role in understanding the cellular mechanisms and activity-based sensing methods. Particularly, ligand directed site-selective labeling of proteins increases their selectivity towards the protein of interest (POI). Many such methods have been reported in the literature. The key disadvantage of this method is after labeling the probe, the ligand occupies the active pocket and makes the POI inactive. Over the last decade, Hamachi et al (45) have developed many ligand-directed chemistries in which the ligand leaves after the covalent bond formation with nucleophilic residue on the POI. These methods keep the protein active in the cellular environment to monitor cellular mechanisms. In this context, CoLDR chemistry-based site-selective labeling of proteins and kept the POI in its active form was developed. Previously, ColDR chemistry was used to release activity-based probes. Herein, similar chemistry to release the ligand after the covalent bond formation was used (FIG. 2).

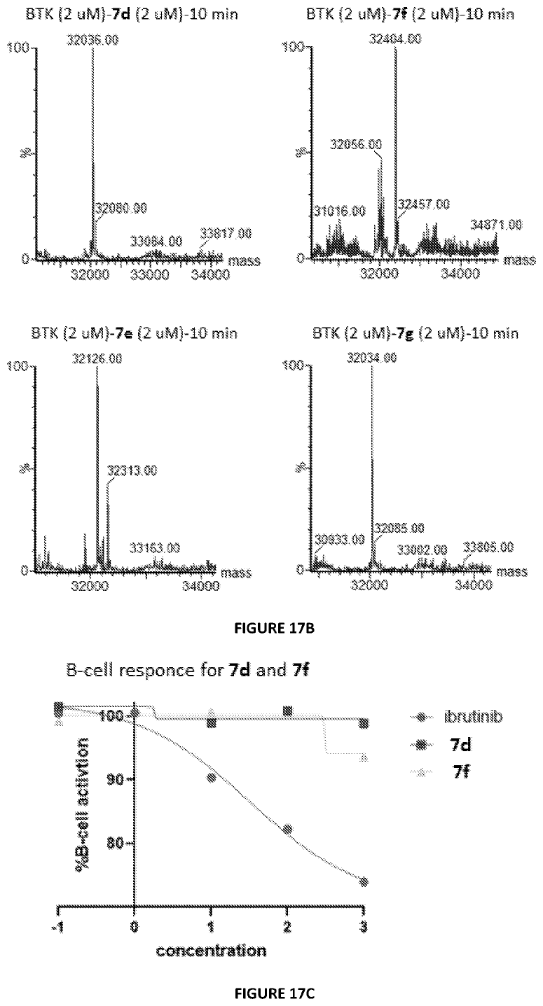

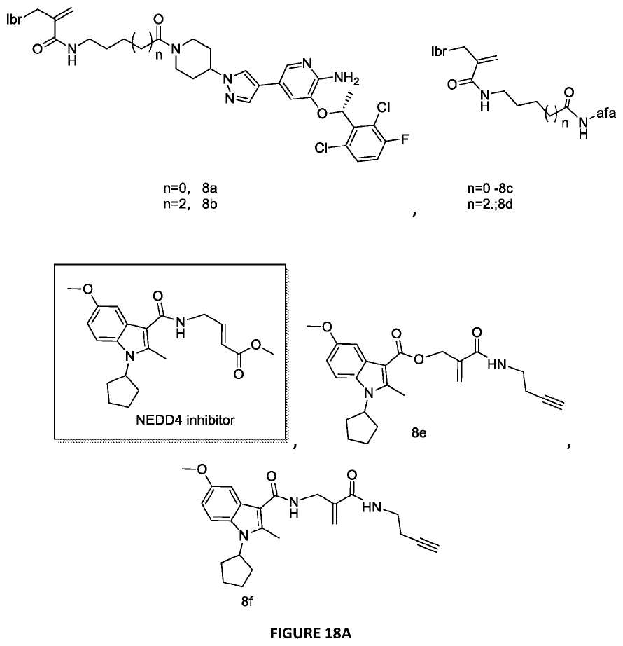

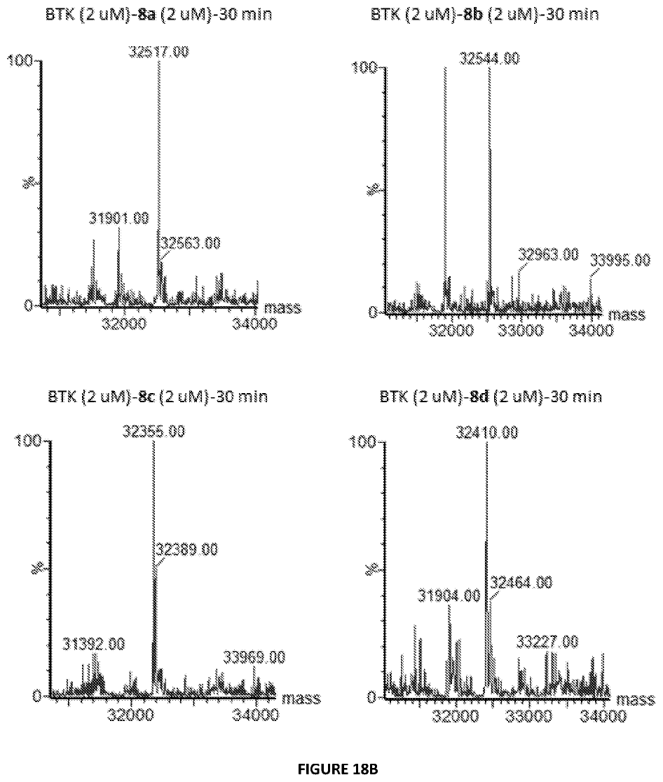

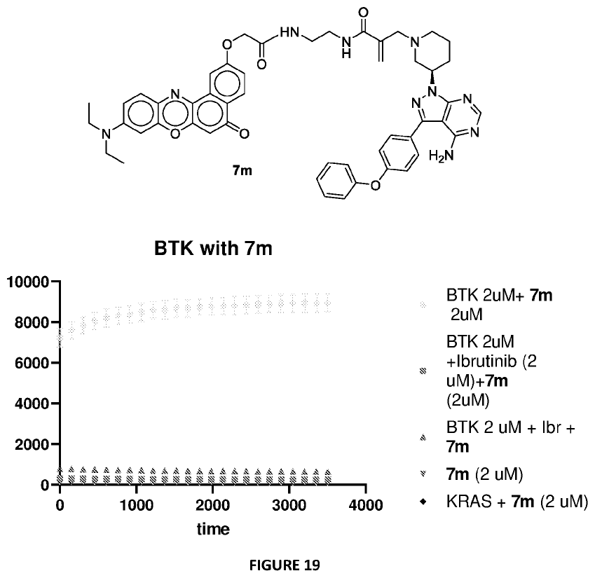

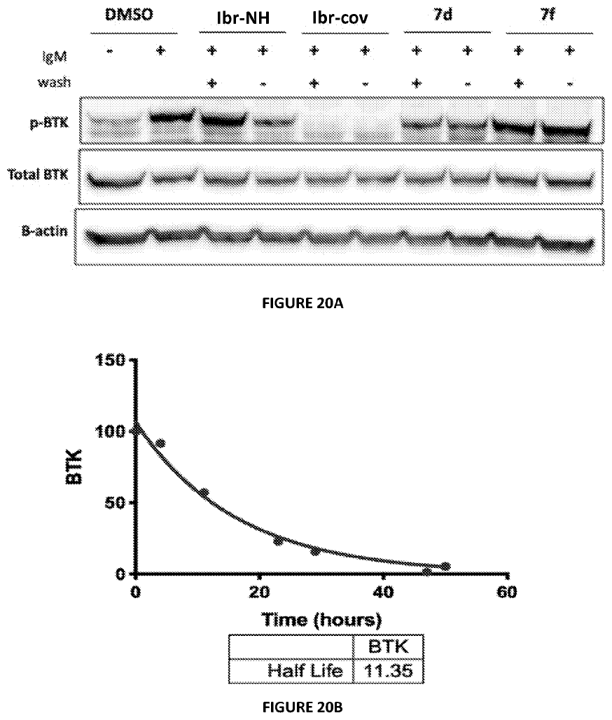

Ibr substituted methacrylamide were synthesized (FIG. 17A) containing an alkyne probe, FAM, and cu-free click probe (FIG. 17A). These compounds showed 100% labeling (2 uM) to BTK (2 uM) with the elimination of Ibr in 1 min. The alkynes and FAM tags on BTK were identified by LC-MS analysis, which shows the m/z corresponds to BTK with tags (FIG. 17B). Further, the compound 7e, which has fluorescein, after incubation with BTK, ran in fluorescent gel and observed the band corresponding mass range.

To assess the compatibility of this chemistry with cellular conditions B cell receptor signaling inhibition was evaluated in primary mouse B cells by two of these compounds 7e and 7f. B cells were incubated (24 h; 37° C.) with the inhibitors at various concentrations, treated with anti-IgM, and activation was assessed by flow cytometry detection of CD86 expression. Both the compounds showed no activity indicating both cellular attachment of the compounds without affecting its activity (FIG. 17C).

### Example 9

### Phosphorylation/Degradation Inducing Reverse CoLDR Tags

The native phosphorylation of BTK even after labelling with alkynes phosphorylated chimeric molecules (PHICs-PMID: 32787262) were prepared. These molecules generally have a binder of a kinase linked to ligands of another protein of interest to which phosphorylation can be done. In this context, Ibr-H substituted methacrylamide linked with ligands like crizotinib (ALK inhibitor) and afatinib were synthesized (FIG. 16A, EGFR inhibitor). It was assumed that compounds 8a and 8b covalently label BTK with elimination of Ibr and reversibly binds with ALK. The close proximity of BTK and ALK can induce the tyrosine phosphorylation in ALK by BTK. Similarly, 8c and 8d can inducetyrosine phosphorylation in EGFR by BTK. When incubating BTK, all the four compounds label BTK within 30 min eliminating Ibr (FIG. 18B).

NEDD 4, an E3 ubiquitin-protein ligase, has a role of selecting specific proteins for conjugation to ubiquitin, and has an acrylate based covalent inhibitor. Labeling of NEDD4 is proposed with another protein ligand using the CoLDR chemistry where NEDD4 inhibitor leaves after labelling and keep the NEDD4 active. Synthesis of an alkyne attached NEDD4 inhibitor (8e, 8f) was preformed to check the engagement of NEDD 4 and leaving its inhibitor ability in cells (FIG. 18A). Coupling of various protein binding moieties to said alkyne will enable degradation of these POIs.

### Example 10

### Ligand Directed Site-Selective Labeling of BTK Active Site Cysteine

To test the engagement of ligand directed chemistry in live cells, Compounds 7a, 7f and 7e in mino cells were tested. Compounds (7a), (7d), and (7f) bind to BTK in cells at 100 nM concentrations. Although (7e), a fluorescein tagged compound, wasn't cell permeable but labelled BTK in lysates at 100 nM.

To assess the activity of the BTK after labelling with alkynes tags in cells, a BTK phosphorylation assay was conducted. It was found that BTK is active and phosphorylated. Labeling of both alkyne probes (7d) and (7f) by leaving ligand out kept the kinase active for the phosphorylation. (FIG. 20a) Further, the CoLDR chemistry was used to check the half-life of the BTK using (7f). The half life of BTK labelled alkyne of (7f) was identified as 11.5 h whereas the half life of BTK with cycloheximide assay was 19.6 h. (FIG. 20b).

### Example 11

### Site Specific Labeling Probes for BTK

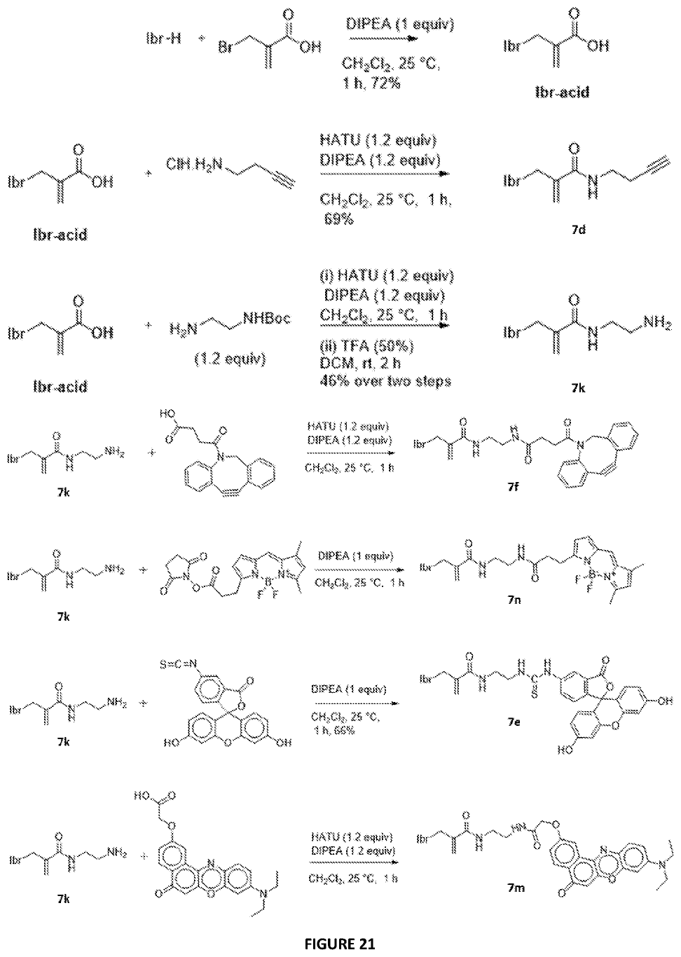

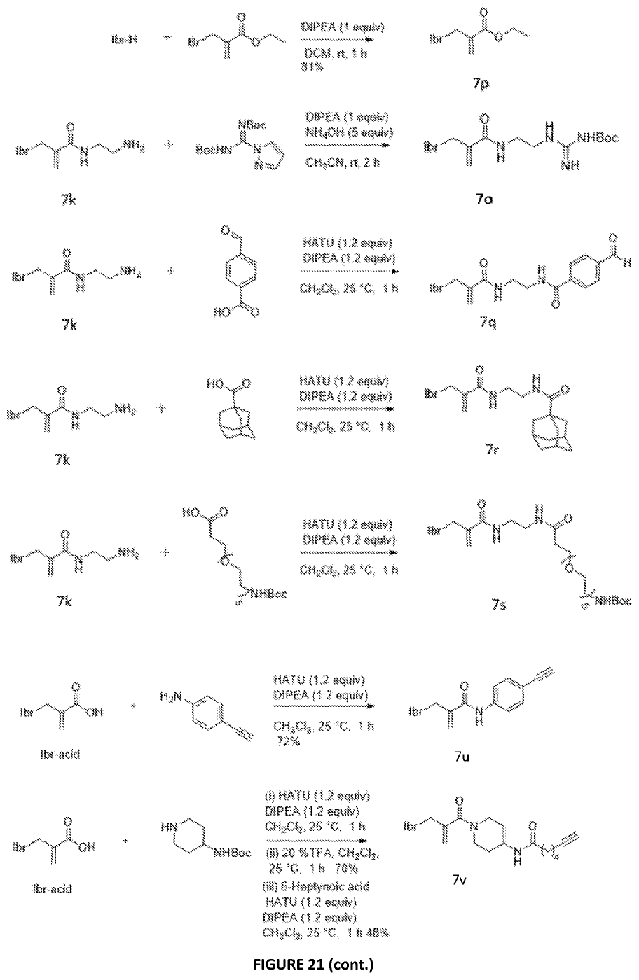

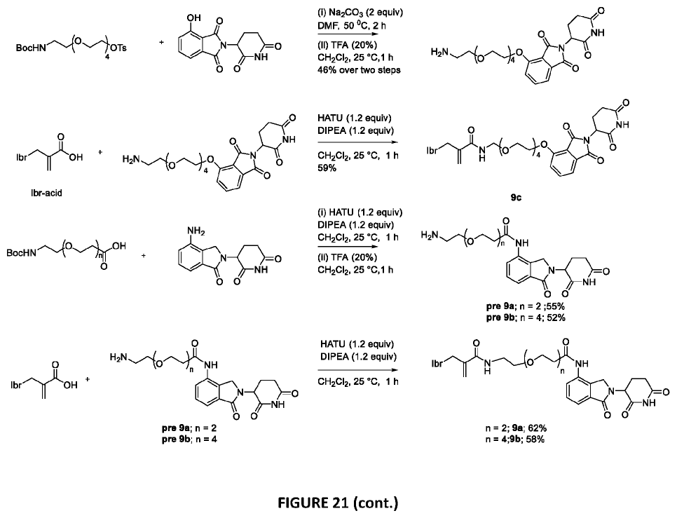

Bruton's tyrosine kinase (BTK), an established drug target for B-cell malignancies, was selected as a model protein for ligand directed site-selective labeling. Ibrutinib, which is a highly potent covalent inhibitor of BTK that binds at its ATP-binding pocket, was used as the ligand to guide the selective labeling of BTK's non-catalytic cysteine 481 (47). The amine precursor for Ibrutinib (Ibr-H; FIG. 17A) contains a piperidine moiety, which can be installed as a hetero substituent on an a-methacrylamide, and thus serve as a leaving group (48). Substituted methacrylamide Ibrutinib analogs (FIG. 17A) which contains various functional probes such as ‘click’ chemistry handles: alkyne (7d) and dibenzyl cyclooctyne (7f), fluorescent dyes (7m, 7n, 7e), hydrophobic tags (7r, 7s), and derivatives of natural amino acid side chains (7c, 7k, 7o, 7g). The synthesis of these probes are described in Example 1 and in FIG. 21.

To assess irreversible labeling and validate the proposed ligand release mechanism, the probes/compounds (2 μM) were incubated with recombinant BTK (2 μM) and monitored the reaction via intact protein liquid chromatography/mass spectrometry (LC/MS). For example, analysis of the reaction with 7n (FIG. 22B) verified that the shift in mass corresponds to labeling BTK with BODIPY and release of Ibr-H (FIG. 22C). All of the tested probes labeled BTK to 100% within 10-120 min at pH 8, 25° C. (FIG. 22D), with an adduct mass corresponding to the probe without ligand.

To verify the site-specificity, BTK incubated with either DMSO or 7d followed by trypsin digestion and analysis of the tryptic peptides by LC/MS/MS was preformed. Cys481 was identified as the site of modification both through MS/MS identification of the 7d modified tryptic peptide (residues 467-487), as well as by depletion of iodoacetamide-labeled 467-487 peptide upon reaction with 7d.

To assess the kinetic parameters of labeling, a time-dependent incubation experiment of BTK (200 nM) was performed with various concentrations of 7d (300-2000 nM, 20 mM Tris, pH 8, 14° C.), resulting in k_{inact}=2.78×10⁻² s⁻¹ and Kᵢ=3.0×10⁻⁷ M under these conditions. These values are similar to previously reported values for Ibrutinib⁵⁴ (k_{inact}=2.70×10⁻² s⁻¹; Kᵢ=5.42×10⁻⁸ M; k_{inact}/Kᵢ=4.98×10⁵) where the reversible binding component is about 5-fold weaker for 1b and k_{inact} is similar.

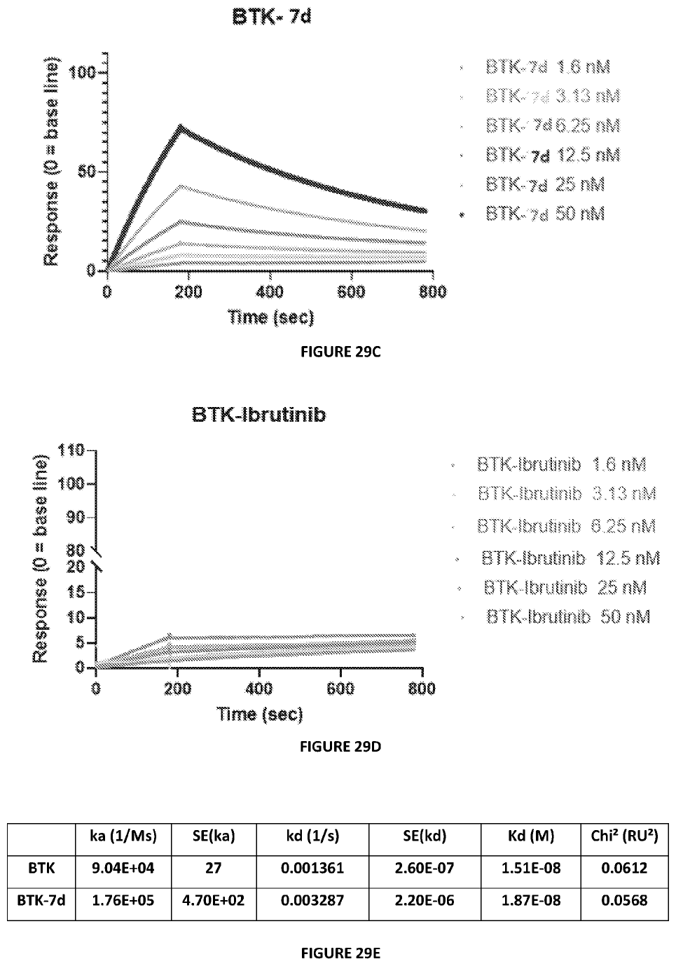

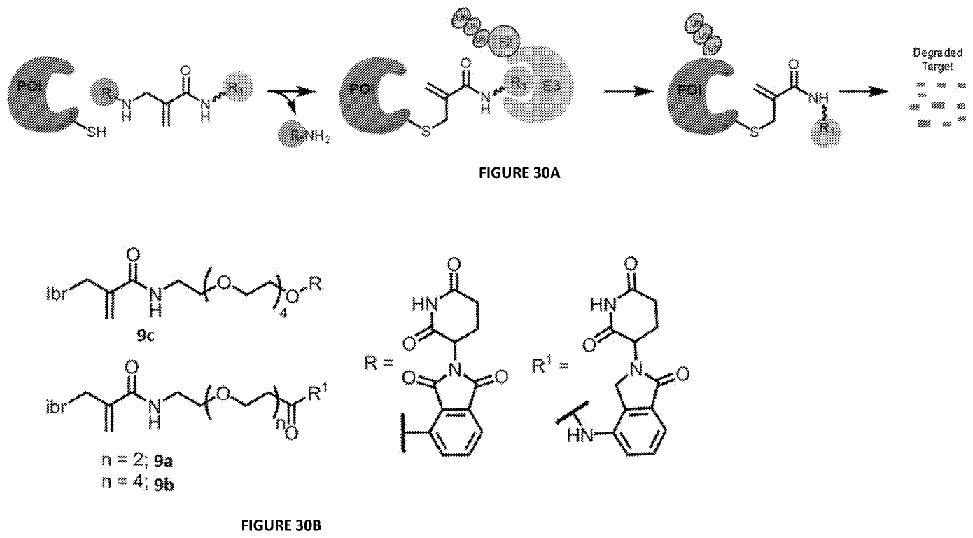

To validate that the binding site of BTK remains vacant following labeling by 7d, a performed surface plasmon resonance (SPR) experiments were performed. A reversible analog of Ibrutinib through a long PEG linker was conjugated to the SPR chip a reversible analog of Ibrutinib through a long PEG linker (9e; FIG. 29A-29D). We then flowed either free BTK (FIG. 29B), 7d labeled BTK (following irreversible labeling we removed excess 7d and Ibr-H via dialysis, see Methods; FIG. 29C) or Ibrutinib labeled BTK (FIG. 29D) at various concentrations over the chip. Free BTK (K_{D}=15 nM) and BTK-7d (K_{D}=18 nM) bind 9e with high affinity (FIG. 29E) whereas BTK-Ibrutinib does not show any binding. This indicates that the labelling of BTK with CoLDR probes does not affect the binding of other reversible ligands.

The stability of BTK labelled with a CoLDR probe was assessed in the presence of reduced glutathione (GSH). BTK (2 μM) was incubated with 7n (2 μM; 30 min; pH 8; 25° C.). The BTK-7n conjugate was then further incubated with GSH (1 mM or 5 mM; 18 h; pH 8; 25° C.). After 18 h, no detachment of the probe from BTK or addition of GSH was observed indicating the stability of this modification to conditions similar to the cellular environment.

Solvatochromic fluorophores possess emission properties that are sensitive to the nature of the local microenvironment which is exploited to study protein structural dynamics and the detection of protein-binding interactions⁴⁹. Recently it was shown that even localization of a solvatochromic fluorophore to a non-specific protein surface can result in ‘turn-on’ fluorescence⁵⁵⁻⁵⁶. However, the presence of bound ligands can impose significant structural changes on the structure of proteins. Compound 7m, which has an environmentally sensitive fluorogenic probe, allowed to develop a turn-on fluorescent probe for BTK in its apo form.

7m has negligible fluorescence in and of itself (Ex/Em=550/620 nm; FIG. 22E). However, upon the addition of BTK (pH 8, 37° C.), the fluorescence intensity of 7m at 550 nm increased 80-fold within seconds, reaching saturation within 5 min (FIG. 22E). Such fast labeling compared to the results reported in FIG. 22D may be the result of the higher temperature at which this experiment was performed. Intact protein LC/MS following the fluorescence measurement showed the expected adduct mass of the fluorophore without the Ibrutinib recognition element, validating covalent binding and the proposed mechanism (FIG. 22F). Pre-incubation with either Ibrutinib or the non-covalent analog of Ibrutinib (Ibr-H), eliminated the fluorescence, indicating that it requires binding at the active site of BTK. Further, the LC/MS chromatogram of these control reactions showed no labelling of 7m in the presence of competitors (FIG. 22F). To assess the selectivity of the probe, it was incubated with an alternative covalent target, K-Ras^{G12C} which did not elicit fluorescence (FIG. 22E). The initial rate of fluorescence generation could be assessed, by reducing the concentration of the reactants 7m (50 nM) with BTK (1 μM) at 30° C.

7m was tested to detect binding events within the active site of BTK. After labelling BTK with 7m, the adduct was incubated with Ibr-H or with Ibrutinib. This resulted in a 2-3 fold decrease of fluorescence, as well as a significant red shift of the emission from 620 nm to 650 nm (FIGS. 28A, 28B). These results indicated that BTK retained the ability to bind the ligands in the active site after being labelled. The change in fluorescence may be due to conformational changes of BTK or in the positioning of the fluorescent probe after binding, resulting in an altered chemical environment. Spectral changes were also observed with BTK pre-labelled with 7n and 7e (FIGS. 28D and 28E).

These spectral changes were followed in a small screen of BTK active site binders. Several BTK active site binders were incubated with 7m labelled BTK and recorded the fluorescence spectra. Interestingly, many compounds shifted the fluorescence spectrum peak from 620 to 650 and/or quenched the fluorescence. Several of the compounds with the most pronounced effects are kinase inhibitors.

### Example 12

### Intrinsic Thiol Reactivity of BTK Probes

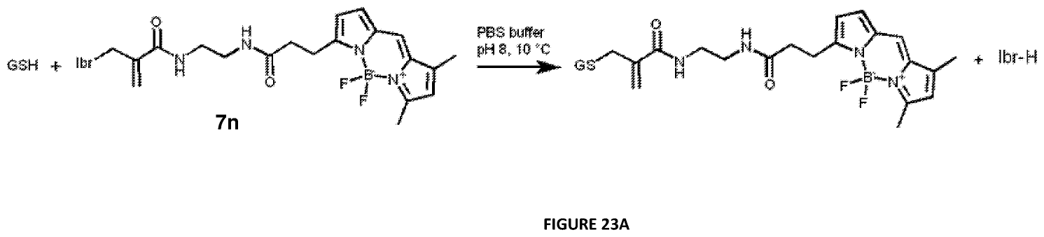

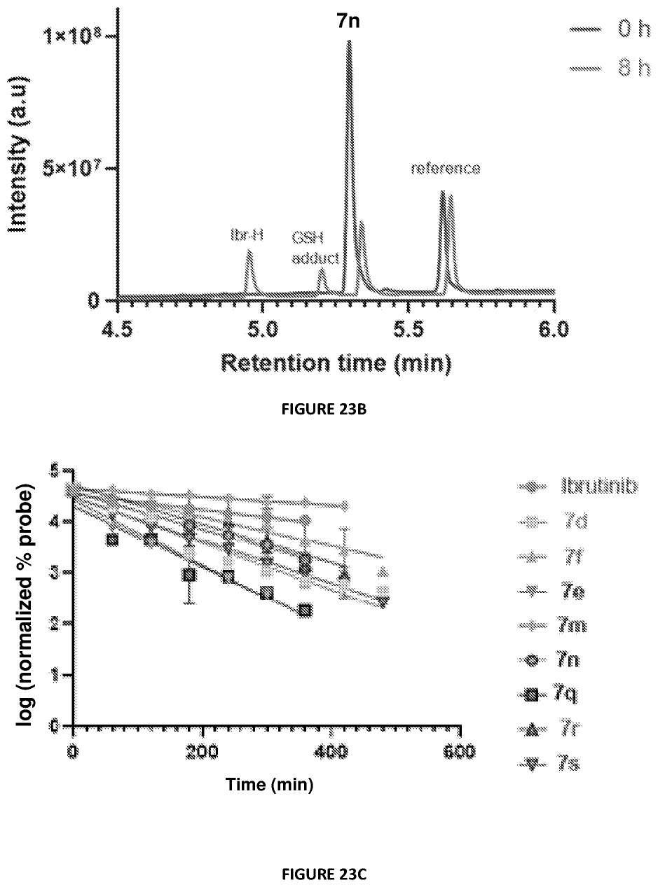

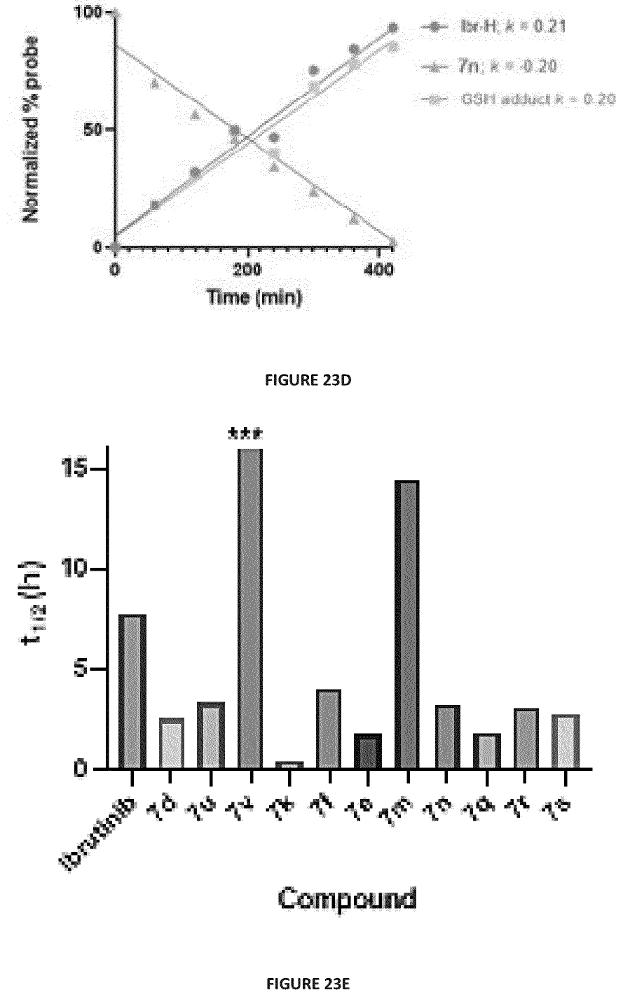

To explore the intrinsic thiol reactivity of these BTK labeling probes, they (7c, 7d, 7k, 7f, 7e, 7m, 7n, 7o, 7q, 7r, 7s) were reacted with reduced glutathione (GSH; 5 mM; PBS buffer at pH 8), as a model thiol and monitored the reaction over time via (LC/MS; FIG. 23A). As an example, analysis of the reaction of Compound 7n after 0 h and 8 h (FIG. 23B), clearly indicates the formation of a substitution product, the release of Ibr-H, and the decrease of starting material. The rates of the release of Ibr-H, formation of the GSH adduct, and depletion of Compound 7n are identical (FIG. 23D), suggesting the release of ligand (Ibr-H) is concomitant with the reaction with GSH. Further, to compare the reactivity of these probes with Ibrutinib, it was measured GSH consumption (t_{1/2}) for all compounds (FIGS. 23C and 23E). Almost all probes show a reactivity within a two-fold rage of Ibrutinib. Most of which are slightly more reactive than Ibrutinib, with the exception of 7m and 7v which is about two-fold less reactive respectively. The ester based 7c is significantly more reactive (t_{1/2}<10 min). It is interesting to compare compounds 7d, 7u, 7v that differ in the nature of the acrylamide amine. The simple primary amine and aniline show moderate reactivity (t_{1/2}=30 min−4 h) towards GSH, whereas 7v with a piperidine moiety shows t_{1/2}.>100 h. This variation in reactivity may help tune the selectivity of these probes. Note that 7m and 7v with the least reactivity towards GSH also showed lower labeling of BTK (FIG. 22D). None of the compounds show decomposition under the GSH reaction conditions.

### Example 13

### CoLDR Labeling is General Across Protein Targets

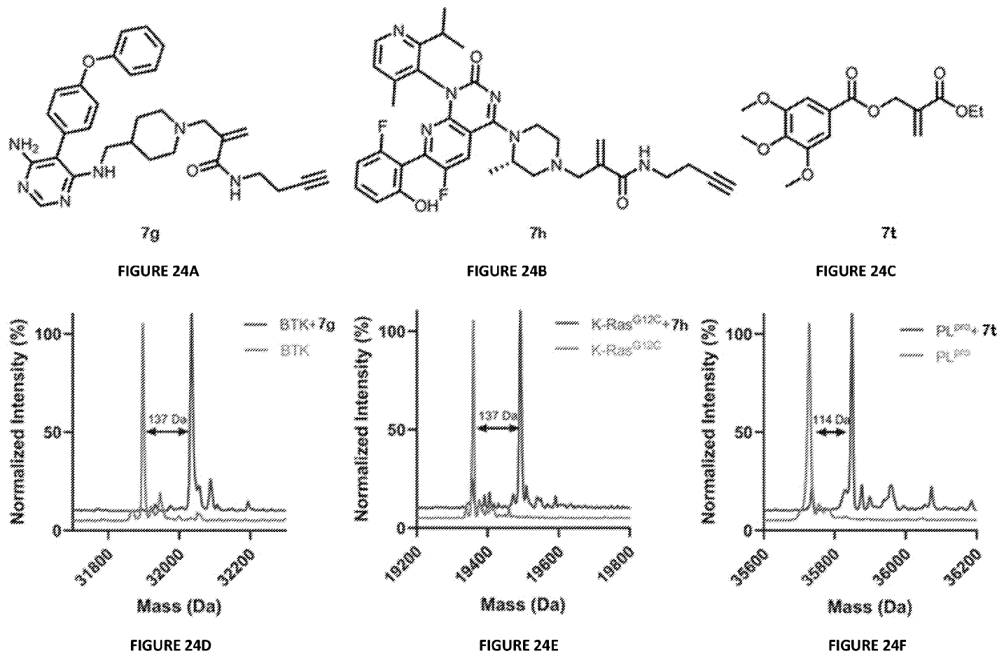

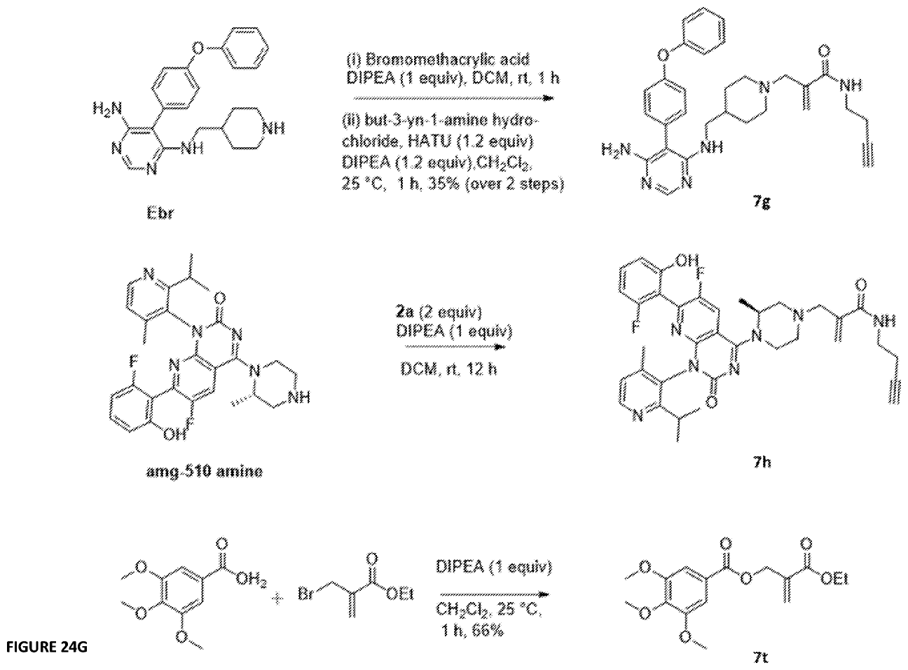

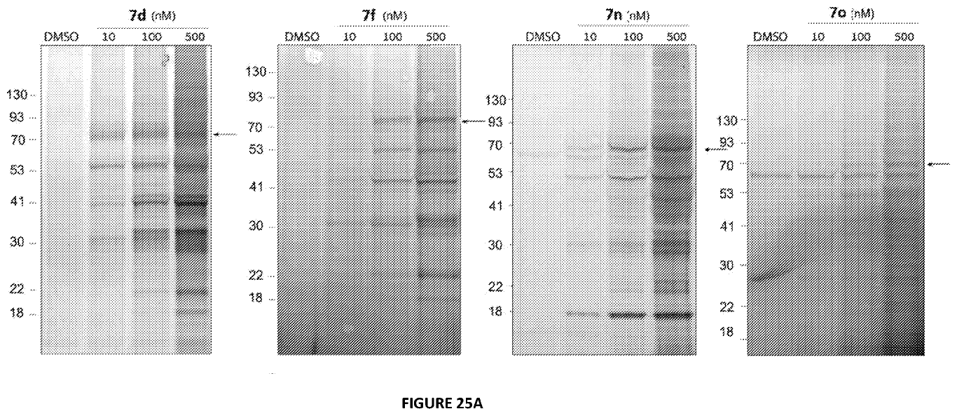

To show the generality of this approach, another ligand of BTK was used: evobrutinib, as well as two other therapeutic targets for which covalent inhibitors were available: K-RAS^{G}12c and the SARS-CoV-2 papain like protease (PL^{pro}) as model systems. An evobrutinib based alkyne probe (7g; FIG. 24A), an AMG-510 based alkyne probe to target K-Ras^{G12C} (7h; FIG. 24B) and an ethyl-acrylate labeling ligand for PL^{pro} based on a covalent ligand were synthesized and previously identified (7t; FIG. 24C). The probes were incubated with their targets (BTK: 2 μM, 10 min, 25° C.; KRas^{G12C}: 10 μM, 16h, 37° C.; PL^{pro}: 2 μM, 16h, 37° C.; all reactions performed at pH=8). All three probes were able to reach 100% single labeling of their target as assessed by LC/MS (FIG. 24D-24F) with the adduct masses corresponding to the alkynes (BTK and KRas^{G12C}) or ethyl acrylate (PL^{pro}). It should be noted that in the case of PL^{pro}, since the cysteine target is the catalytic residue, it is expected that this modification to also inhibit the enzyme.

### Example 14

### Ligand Directed Site-Selective Labeling of BTK in Cells

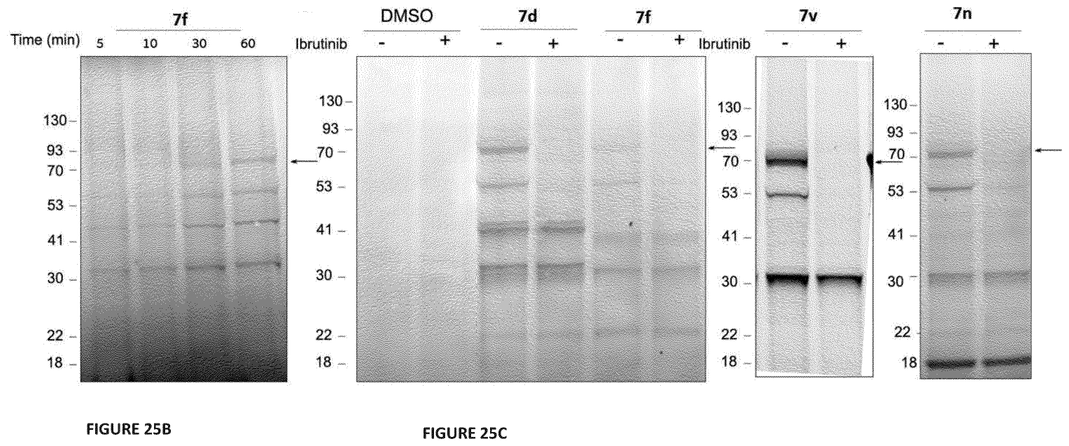

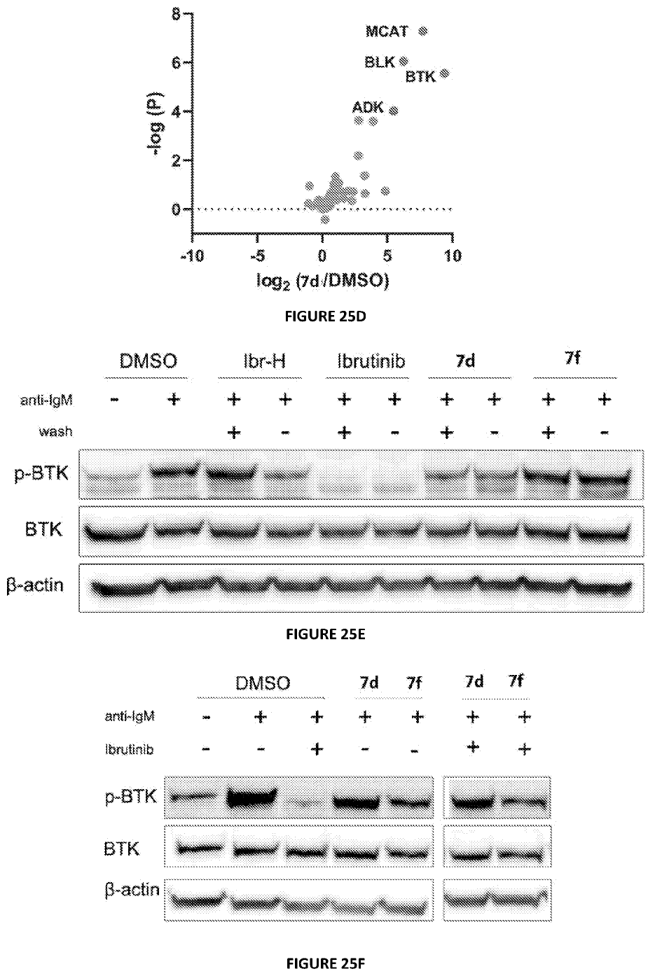

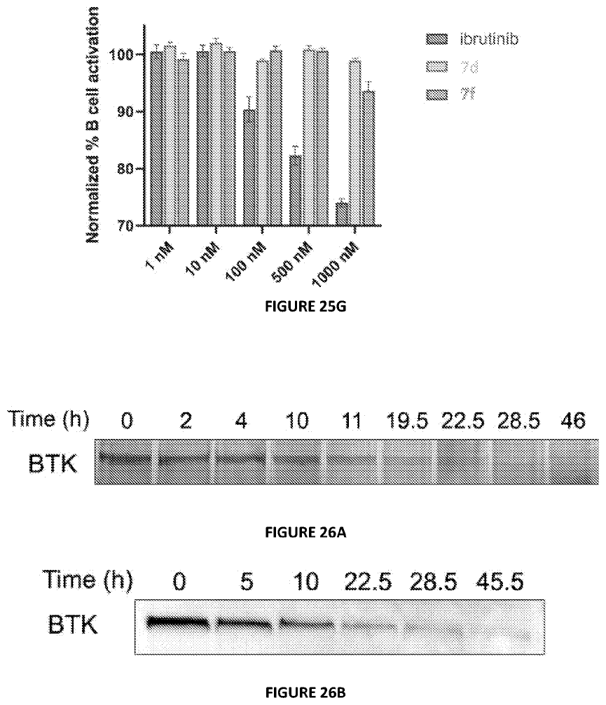

In addition to the in vitro labeling of BTK by the probes described herein, their engagement in cells and proteomic selectivity were tested. Mino B cells were incubated with various probes containing different tags, such as an alkyne (7d, 7u, 7v), dibenzocyclooctyne (7f), and the fluorescent dyes fluorescein (7e), nile red (7m), and BODIPY (7n), and used in-gel fluorescence (following CuAAC of TAMRA-N₃ to the alkyne tags) to image their labeling profiles. Probes 7d and 7n showed robust labeling even at a concentration of 10 nM (FIG. 25A) whereas 7v labelled BTK with more selectivity (FIG. 25C). 7f and 7m labelled BTK at a concentration of 100 nM (FIG. 25A) and 7e did not label BTK in live cells. Negatively charged fluorophores such as fluorescein have known permeability issues. Indeed, in lysate 7e was able to label BTK at a concentration of 100 nM (FIG. 25). To assess the kinetics of the cellular labelling, we followed the time-dependent labelling by 7f which showed robust labelling of BTK within 30-60 min (FIG. 25B).

To validate the molecular target of the probes, a competition experiment was performed, where the cells were pre-incubated with Ibrutinib prior to labelling with the probes (FIG. 25C). This experiment confirmed BTK labelling as Ibrutinib completely competed for the labelling of the band at ˜70 kDa, as well as some of the off-targets. It is interesting to note that some off-targets were not competed by Ibrutinib, indicating these are new off-targets specific to our probes (FIG. 25C). To identify the off-targets of these probes, we performed a pull-down proteomics experiment in Mino cells (FIG. 25D) using 7d. Cells were treated with either DMSO, 7d (100 nM), or pre-treated with Ibrutinib and then with 7d. Biotin was conjugated to the alkyne via CuAAC, and avidin beads were used for enrichment. BLK, MCAT and ADK were found as off-targets for probe 7d (FIG. 25D). ADK (40.5 kDa) and MCAT (Also known as SLC25A20; 33 kDa) correspond to the two bands seen in the gel (FIG. 25C) that are not competed by Ibrutinib. Both were abundant proteins in the cell which may explain probe binding. Overall very few off-targets were detected for all probes at the lower concentration.

### Example 15

### BTK Labeling Preserves its Enzymatic Activity

In order to examine the effect of BTK modification by these probes, on its activity, activity assays were performed in both Mino and primary B cells. Mino cells were incubated (1 h) with probes 7d, 7f, 7m and 7n to allow labeling, followed by BTK activation using anti-human IgM. BTK's autophosphorylation was followed by western blot to assess its activity. While Ibrutinib completely abolished BTK autophosphorylation, BTK remained active after labeling with all four probes. 7f, 7m, and 7n in particular did not affect the activity (FIG. 25E). This effect was indifferent to washing of the cells, which abolished inhibition of the BTK reversible inhibitor Ibr-H, but not that of Ibrutinib (FIG. 25E). Further, to ensure that the activity did not originate from unlabeled BTK, Mino cells were treated with high concentrations of 7d, 7f, 7m and 7n (1 μM) for 2 hours and then incubated with 100 nM Ibrutinib for 45 min before activation with IgM. While Ibrutinib alone completely inhibited BTK's activity, we show that all CoLDR probes can rescue this inhibition. Compounds 7n and 7m, do show some reduction in phosphorylation upon Ibrutinib incubation, indicating incomplete BTK labeling in cells. When the concentration of 7m was increased (5 μM; 4 h incubation) adding Ibrutinib no longer reduced the activity. The fact that BTK's activity remains suggests that the labeled fraction remains active (FIG. 25F). In addition, the effect of these probes on B cell receptor (BCR) signaling in primary mouse B cells were measured. Mouse splenic cells were isolated and treated for 24 h with a dose-response of Ibrutinib, 7d, and 7f, and B cell activation in response to stimulation with anti-IgM was measured by following the expression of CD86. In contrast to Ibrutinib, both probes did not inhibit the activation of B cells, suggesting they do not only preserve BTK autophosphorylation but also do not interfere with its downstream signaling (FIG. 25G).

### Example 16

### BTK Half-Life Determination Using CoLDR Probes

As presented in Example 15, the labeling by 7f does not inhibit its native phosphorylation of BTK and its downstream signalling, this probe was used to measure BTK's half-life in the native cellular environment. For that purpose, Mino cells were incubated for 1 hour with 7f to label BTK, followed by washing to ensure that newly synthesized BTK will not be labeled. Cells were then harvested at different time-points, lysed, and “clicked” using a Cu-free reaction by the addition of TAMRA-azide. BTK abundance was followed by in-gel fluorescence, which allowed quantification and the half-life determination (FIG. 26A). The average half-life of BTK measured with 7f was 10.2±2.0 hours, which is similar to its half-life measured with the traditional cycloheximide (CHX) assay (FIG. 26B, 26C, 26D), but did not require an antibody, western blotting, and importantly did not perturb the cell translation machinery.

It should be noted that the loss of 7f signal is due to a decrease in BTK protein levels and not, for example, probe decomposition, since several 7f off-targets exhibited much longer half-lives, indicating the probe is stable over these time scales.

### Example 17

### BTK Tagging does not Interfere with PROTAC Binding and Ternary Complex Formation

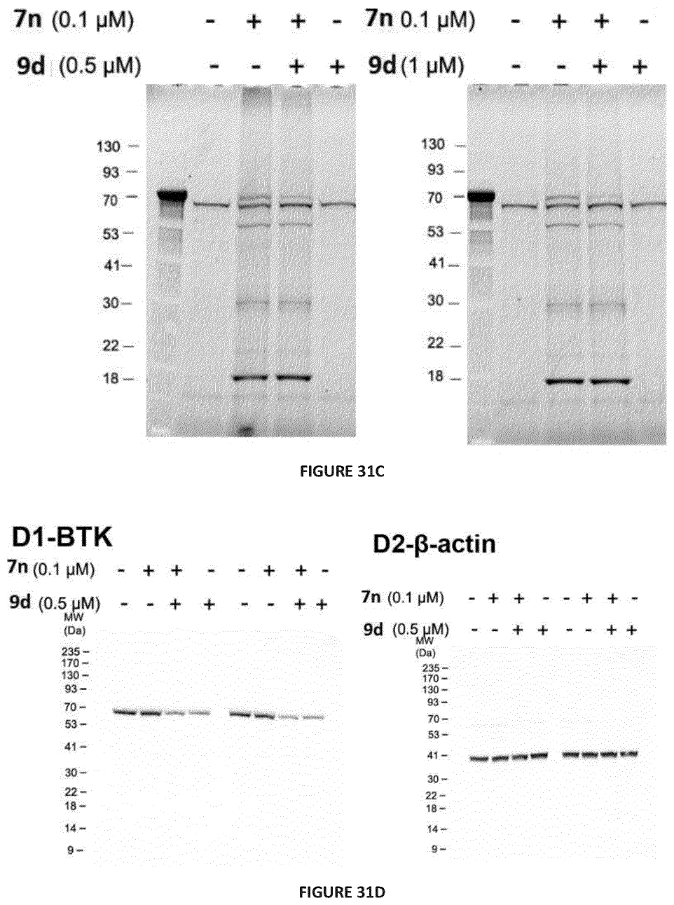

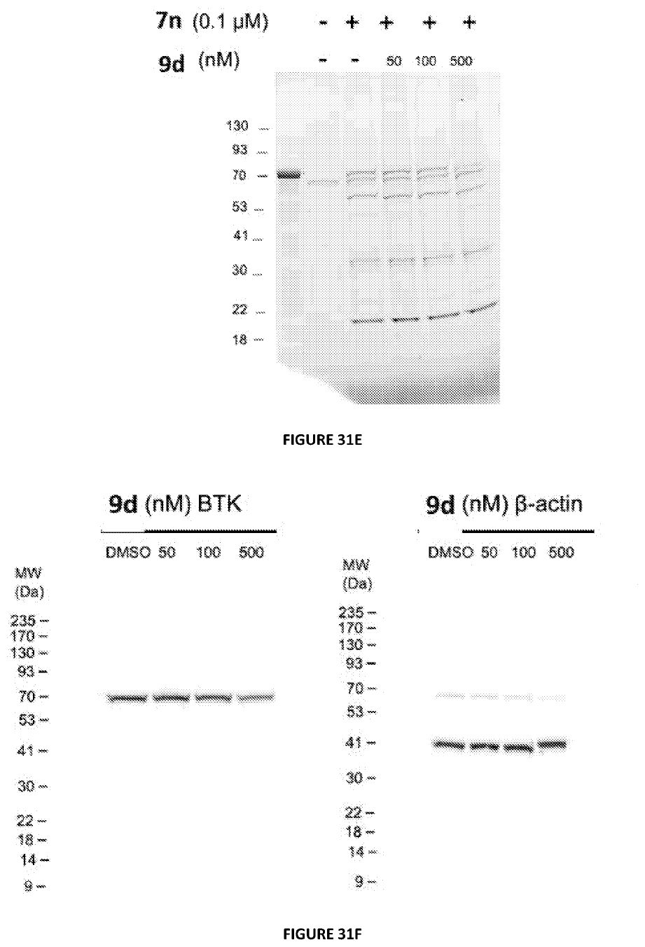

Proteolysis targeting chimeras (PROTACs) are a popular modality to induce selective degradation of cellular proteins. It was shown, that tagging BTK with an alkyne allowed to follow its natural degradation in the cell. The induced targeted degradation was followed by a BTK PROTAC. To do so, we incubated Mino cells with fluorescent probe 7n (100 nM) for 1 h then washed the cells and incubated them with a non-covalent BTK PROTAC 9d⁴⁶ (FIG. 31B) for 2 h and measured BTK degradation using both in-gel fluorescence (FIGS. 26E and 31C) and western blotting (FIG. 31D). Interestingly, degradation of BTK quantified by gel fluorescence (75% at 1 μM, 55% at 0.5 μM) closely corresponds to the quantification by the western blot (71% at 1 μM, 55% at 0.5 μM). This suggests the PROTAC mediated degradation can be followed using in-gel fluorescence by pre-labelling the target with a CoLDR fluorescent tag. Importantly, in the absence of 7n, PROTAC 9d degraded 65% of the protein at 0.5 μM and 1 μM. Both are similar to the degradation by 9d in the presence of the fluorescence tag. Almost no degradation has been observed at lower concentrations of 9d (50 nM and 100 nM) both in the presence and absence of 7n (FIGS. 31E and 31F). Altogether this data suggests the fluorescent tag does not interfere with the binding of a non-covalent PROTAC nor with the formation of a ternary complex with CRBN E3 ligase.

### Example 18

### CoLDR Chemistry Allows the Installation of a Degradation Handle

Small molecule binders are known to thermodynamically stabilize their target proteins, which may also translate to improved cellular stability to degradation.

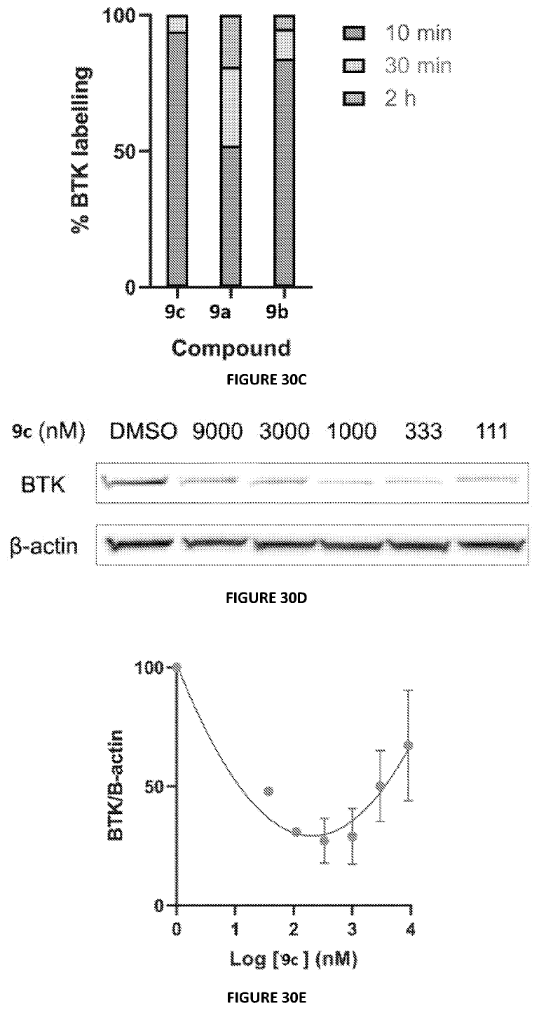

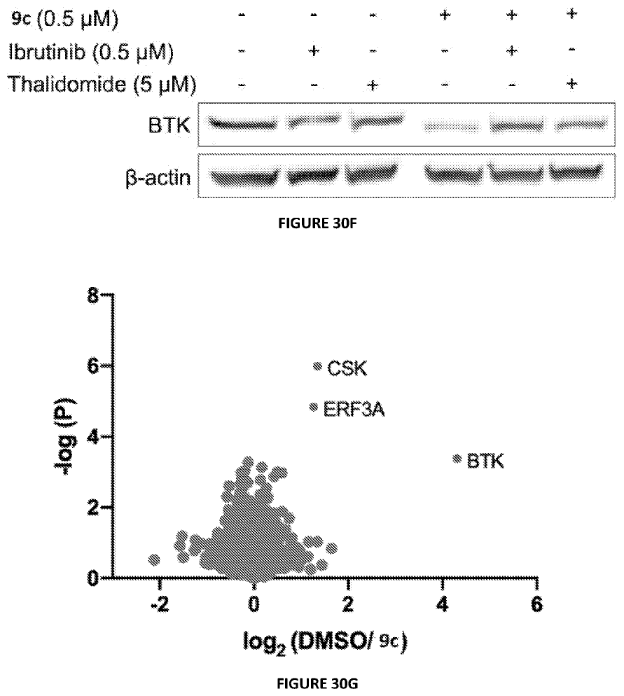

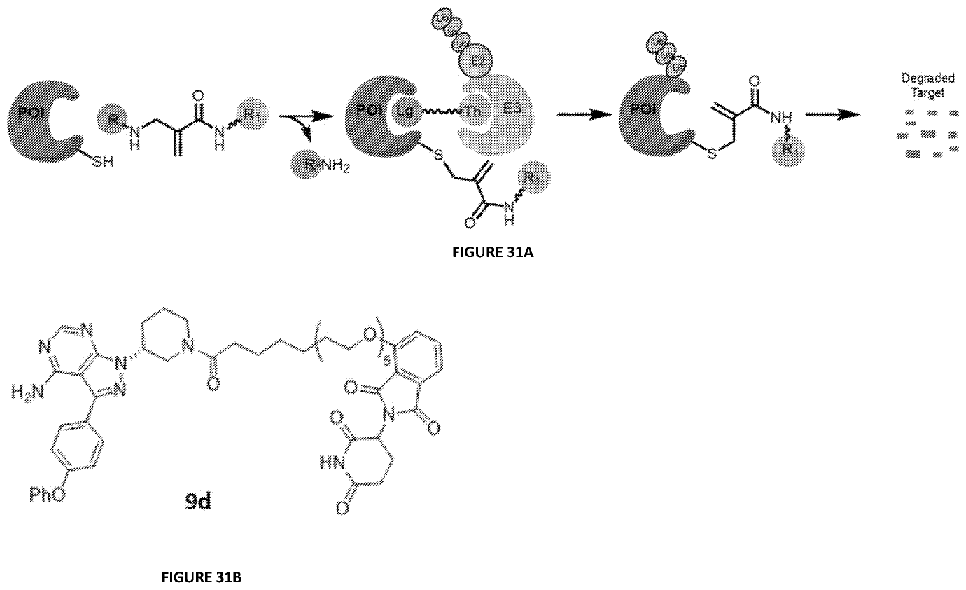

Three CoLDR PROTACs were designed that utilize Ibr-H as a leaving group, to install a CRBN binder (thalidomide/lenalidomide) through a PEG linker onto BTK (FIG. 30B). The synthesis of these compounds is by coupling thalidomide/lenalidomide PEG amine with Ibr-carboxylic acid (FIG. 21). We first assessed BTK labelling by these PROTACs (2 μM BTK, 2 μM PROTAC; pH 8, 25° C.). All three PROTACs labelled BTK by more than 80% within 30 min (FIG. 30C). We then assessed if they can induce BTK degradation in Mino cells. 9c proved the best degrader, with a DC₅₀<100 nM (11.4 nM according to the polynomial fit; FIGS. 30D, 30E). To validate the degradation mechanism of 9c, we pre-treated Mino cells with either Ibrutinib or thalidomide-OH, before incubation with the PROTAC. Both were able to rescue the degradation suggesting it was mediated by binding to BTK and to CRBN (FIG. 30F).

Finally, the proteomic selectivity of 9c was assessed by quantitative label free proteomics (FIG. 30G). Out of the proteins identified and quantified in both DMSO and 9c-treated samples, only three proteins were depleted by more than 50% with ap-value <0.01. The most prominent target was BTK, which was depleted more than 16-fold. A prominent off-target we observed was CSK, a non-covalent off-target of Ibrutinib, which was depleted a little more than 50%. However, depletion of CSK was small relative to values observed for other BTK PROTACs that engaged their target purely noncovalently, indicating that covalent binding plays an important role in target recruitment.

The second major off-target, Erf3A (also known as GSPT1) is a known target for IMiD-CRBN binders. None of the off-targets enriched by 7d (FIG. 25D) was detected as a degradation target of 9c. Very few proteins were identified and quantified only in one set of the samples, precluding their quantification. Three proteins were observed in DMSO-treated samples but were not detected in the 9c treated samples, among them the prominent ibrutinib off-target BLK.

While certain features and uses thereof have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure herein.

### REFERENCES

1. Griffin, B. A.; Adams, S. R.; Tsien, R. Y. Specific Covalent Labeling of Recombinant Protein Molecules inside Live Cells. Science 1998, 281 (5374), 269-272.
2. Xue, L.; Karpenko, I. A.; Hiblot, J.; Johnsson, K. Imaging and Manipulating Proteins in Live Cells through Covalent Labeling. Nat. Chem. Biol. 2015, 11 (12), 917-923.
3. Nischan, N.; Hackenberger, C. P. R. Site-Specific PEGylation of Proteins: Recent Developments. J. Org. Chem. 2014, 79 (22), 10727-10733.
4. Tsai, Y.-H.; Essig, S.; James, J. R.; Lang, K.; Chin, J. W. Selective, Rapid and Optically Switchable Regulation of Protein Function in Live Mammalian Cells. Nat. Chem. 2015, 7 (7), 554-561.
5. Yang, S.-T.; Lim, S. I.; Kiessling, V.; Kwon, I.; Tamm, L. K. Site-Specific Fluorescent Labeling to Visualize Membrane Translocation of a Myristoyl Switch Protein. Sci. Rep. 2016, 6, 32866.
6. Zimmer, M. Green Fluorescent Protein (GFP): Applications, Structure, and Related Photophysical Behavior. Chem. Rev. 2002, 102 (3), 759-781.
7. Los, G. V.; Encell, L. P.; McDougall, M. G.; Hartzell, D. D.; Karassina, N.; Zimprich, C.; Wood, M. G.; Learish, R.; Ohana, R. F.; Urh, M.; Simpson, D.; Mendez, J.; Zimmerman, K.; Otto, P.; Vidugiris, G.; Zhu, J.; Darzins, A.; Klaubert, D. H.; Bulleit, R. F.; Wood, K. V. HaloTag: A Novel Protein Labeling Technology for Cell Imaging and Protein Analysis. ACS Chem. Biol. 2008, 3 (6), 373-382.
8. Zeng, M.; Xiong, Y.; Safaee, N.; Nowak, R. P.; Donovan, K. A.; Yuan, C. J.; Nabet, B.; Gero, T. W.; Fern, F.; Li, L.; Gondi, S.; Ombelets, L. J.; Quan, C.; Janne, P. A.; Kostic, M.; Scott, D. A.; Westover, K. D.; Fischer, E. S.; Gray, N. S. Exploring Targeted Degradation Strategy for Oncogenic KRASG12C. Cell Chem Biol 2020, 27 (1), 19-31.e6.
9. Meyer, T.; Begitt, A.; Vinkemeier, U. Green Fluorescent Protein-Tagging Reduces the Nucleocytoplasmic Shuttling Specifically of Unphosphorylated STAT1. FEBS J. 2007, 274 (3), 815-826.
10. Barth, S.; Glick, D.; Macleod, K. F. Autophagy: Assays and Artifacts. J. Pathol. 2010, 221 (2), 117-124.
11. Lang, K.; Chin, J. W. Cellular Incorporation of Unnatural Amino Acids and Bioorthogonal Labeling of Proteins. Chem. Rev. 2014, 114 (9), 4764-4806.
12. Sletten, E. M.; Bertozzi, C. R. Bioorthogonal Chemistry: Fishing for Selectivity in a Sea of Functionality. Angew. Chem. Int. Ed Engl. 2009, 48 (38), 6974-6998.
13. Spicer, C. D.; Davis, B. G. Selective Chemical Protein Modification. Nat. Commun. 2014, 5, 4740.
14. Zhang, C.; Welbom, M.; Zhu, T.; Yang, N. J.; Santos, M. S.; Van Voorhis, T.; Pentelute, B. L. II-Clamp-Mediated Cysteine Conjugation. Nat. Chem. 2016, 8 (2), 120-128.
15. MacDonald, J. I.; Munch, H. K.; Moore, T.; Francis, M. B. One-Step Site-Specific Modification of Native Proteins with 2-Pyridinecarboxyaldehydes. Nat. Chem. Biol. 2015, 11 (5), 326-331.
16. Boutureira, O.; Bernardes, G. J. L. Advances in Chemical Protein Modification. Chem. Rev. 2015, 115 (5), 2174-2195.
17. Matos, M. J.; Oliveira, B. L.; Martinez-Saez, N.; Guerreiro, A.; Cal, P. M. S. D.; Bertoldo, J.; Maneiro, M.; Perkins, E.; Howard, J.; Deery, M. J.; Chalker, J. M.; Corzana, F.; Jiménez-Osés, G.; Bernardes, G. J. L. Chemo- and Regioselective Lysine Modification on Native Proteins. J. Am. Chem. Soc. 2018, 140 (11), 4004-4017.
18. Adusumalli, S. R.; Rawale, D. G.; Singh, U.; Tripathi, P.; Paul, R.; Kalra, N.; Mishra, R. K.; Shukla, S.; Rai, V. Single-Site Labeling of Native Proteins Enabled by a Chemoselective and Site-Selective Chemical Technology. J. Am. Chem. Soc. 2018, 140 (44), 15114-15123.
19. Purushottam, L.; Adusumalli, S. R.; Singh, U.; Unnikrishnan, V. B.; Rawale, D. G.; Gujrati, M.; Mishra, R. K.; Rai, V. Single-Site Glycine-Specific Labeling of Proteins. Nat. Commun. 2019, 10 (1), 2539.
20. Chen, G.; Heim, A.; Riether, D.; Yee, D.; Milgrom, Y.; Gawinowicz, M. A.; Sames, D.

Reactivity of Functional Groups on the Protein Surface: Development of Epoxide Probes for Protein Labeling. J. Am. Chem. Soc. 2003, 125 (27), 8130-8133.
21. Amaike, K.; Tamura, T.; Hamachi, I. Recognition-Driven Chemical Labeling of Endogenous Proteins in Multi-Molecular Crowding in Live Cells. Chem. Commun. 2017, 53 (88), 11972-11983.
22. Chen, J.; Wang, X.; He, F.; Pan, Z. Development of a Selective Labeling Probe for Bruton's Tyrosine Kinase Quantification in Live Cells. Bioconjug. Chem. 2018, 29 (5), 1640-1645.
23. Zhang, Q.; Liu, H.; Pan, Z. A General Approach for the Development of Fluorogenic Probes Suitable for No-Wash Imaging of Kinases in Live Cells. Chem. Commun. 2014, 50 (97), 15319-15322.
24. Zhao, Z. & Bourne, P. E. Progress with covalent small-molecule kinase inhibitors. Drug Discov. Today 23, 727-735 (2018).
25. Liu, Q. et al. Developing irreversible inhibitors of the protein kinase cysteinome. Chemistry and Biology 20, 146-159 (2013).
26. Baillie, T. A. Targeted Covalent Inhibitors for Drug Design. Angew. Chem. Int. Ed Engl. 55, 13408-13421 (2016).
27. Singh, J., Petter, R. C., Baillie, T. A. & Whitty, A. The resurgence of covalent drugs. Nat. Rev. Drug Discov. 10, 307-317 (2011).
28. Shannon, D. A. & Weerapana, E. Covalent protein modification: the current landscape of residue-specific electrophiles. Curr. Opin. Chem. Biol. 24, 18-26 (2015).
29. Chen, D., Guo, D., Yan, Z. & Zhao, Y. Allenamide as a bioisostere of acrylamide in the design and synthesis of targeted covalent inhibitors. Medchemcomm 9, 244-253 (2018).
30. Mons, E. et al. The Alkyne Moiety as a Latent Electrophile in Irreversible Covalent Small Molecule Inhibitors of Cathepsin K. J. Am. Chem. Soc. 141, 3507-3514 (2019).
31. McAulay, K. et al. Alkynyl Benzoxazines and Dihydroquinazolines as Cysteine Targeting Covalent Warheads and Their Application in Identification of Selective Irreversible Kinase Inhibitors. J Am. Chem. Soc. 142, 10358-10372 (2020).
32. Kojima, H. et al. Cyclization Reaction-Based Turn-on Probe for Covalent Labeling of Target Proteins. Cell Chem Biol 27, 334-349.e11 (2020). 33. Fujishima, S.-H., Yasui, R., Miki, T., Ojida, A. & Hamachi, I. Ligand-directed acyl imidazole chemistry for labeling of membrane-bound proteins on live cells. J Am. Chem. Soc. 134, 3961-3964 (2012).
34. Tamura, T. & Hamachi, I. Chemistry for Covalent Modification of Endogenous/Native Proteins: From Test Tubes to Complex Biological Systems. J. Am. Chem. Soc. 141, 2782-2799 (2019).
35. Tamura, T. et al. Rapid labelling and covalent inhibition of intracellular native proteins using ligand-directed N-acyl-N-alkyl sulfonamide. Nat. Commun. 9, 1870 (2018).
36. Tamura, T. et al. Affinity-Guided Oxime Chemistry for Selective Protein Acylation in Live Tissue Systems. J. Am. Chem. Soc. 139, 14181-14191 (2017).
37. Rautio, J., Meanwell, N. A., Di, L. & Hageman, M. J. The expanding role of prodrugs in contemporary drug design and development. Nat. Rev. Drug Discov. 17, 559-587 (2018).
38. Najjar, A., Najjar, A. & Karaman, R. Newly Developed Prodrugs and Prodrugs in Development; an Insight of the Recent Years. Molecules 25, (2020).
39. Gnaim, S. & Shabat, D. Activity-Based Optical Sensing Enabled by Self-Immolative Scaffolds: Monitoring of Release Events by Fluorescence or Chemiluminescence Output. Acc. Chem. Res. 52, 2806-2817 (2019).
40. Giang, I., Boland, E. L. & Poon, G. M. K. Prodrug applications for targeted cancer therapy. AAPS J. 16, 899-913 (2014).
41. Zhang, X., Li, X., You, Q. & Zhang, X. Prodrug strategy for cancer cell-specific targeting: A recent overview. Eur. J. Med. Chem. 139, 542-563 (2017).
42. Kang, J. J., Toma, I., Sipos, A. & Peti-Peterdi, J. From in vitro to in vivo: imaging from the single cell to the whole organism. Curr. Protoc. Cytom. Chapter 12, Unit 12.12 (2008).
43. Dias, G. G., King, A., de Moliner, F., Vendrell, M. & da Silva Júnior, E. N. Quinone-based fluorophores for imaging biological processes. Chem. Soc. Rev. 47, 12-27 (2018).
44. Haeusler, D., Decristoforo, C., Frost, J., Gobalakrishnan, S. & Huang, Y. Y. Molecular Imaging: In Vivo Agents for the Diagnosis and Treatment of Cancer. Contrast Media Mol. Imaging 2018, U.S. Pat. No. 8,541,915 (2018).
45. Tamura, T., Tsukiji, S., & Hamachi, I. Native FKBP12 engineering by ligand-directed tosyl chemistry: labeling properties and application to photo-cross-linking of protein complexes in vitro and in living cells. Journal of the American Chemical Society, 134(4), 2216-2226 (2012).
46. Gabizon, R.; Shraga, A.; Gehrtz, P.; Livnah, E.; Shorer, Y.; Gurwicz, N.; Avram, L.; Unger, T.; Aharoni, H.; Albeck, S.; Brandis, A.; Shulman, Z.; Katz, B.-Z.; Herishanu, Y.; London, N. Efficient Targeted Degradation via Reversible and Irreversible Covalent PROTACs. J Am. Chem. Soc. 2020. https://doi.org/10.1021/jacs.9b13907.
47. Pan, Z.; Scheerens, H.; Li, S.-J.; Schultz, B. E.; Sprengeler, P. A.; Burrill, L. C.; Mendonca, R. V.; Sweeney, M. D.; Scott, K. C. K.; Grothaus, P. G.; Jeffery, D. A.; Spoerke, J. M.; Honigberg, L. A.; Young, P. R.; Dalrymple, S. A.; Palmer, J. T. Discovery of Selective Irreversible Inhibitors for Bruton's Tyrosine Kinase. ChemMedChem 2007, 2 (1), 58-61.
48. Reddi, R. N.; Resnick, E.; Rogel, A.; Rao, B. V.; Gabizon, R.; Goldenberg, K.; Gurwicz, N.; Zaidman, D.; Plotnikov, A.; Barr, H.; Shulman, Z.; London, N. Tunable Methacrylamides for Covalent Ligand Directed Release Chemistry. J. Am. Chem. Soc. 2021, 143 (13), 4979-4992.
49. Tsukiji, S.; Miyagawa, M.; Takaoka, Y.; Tamura, T.; Hamachi, I. Ligand-Directed Tosyl Chemistry for Protein Labeling in Vivo. Nat. Chem. Biol. 2009, 5 (5), 341-343.
50. Takaoka, Y.; Nishikawa, Y.; Hashimoto, Y.; Sasaki, K.; Hamachi, I. Ligand-Directed Dibromophenyl Benzoate Chemistry for Rapid and Selective Acylation of Intracellular Natural Proteins. Chem. Sci. 2015, 6 (5), 3217-3224.
51. Matsuo, K.; Nishikawa, Y.; Masuda, M.; Hamachi, I. Live-Cell Protein Sulfonylation Based on Proximity-Driven N-Sulfonyl Pyridone Chemistry. Angew. Chem. Int. Ed Engl. 2018, 57 (3), 659-662.
52. Kiyonaka, S.; Sakamoto, S.; Wakayama, S.; Morikawa, Y.; Tsujikawa, M.; Hamachi, I. Ligand-Directed Chemistry of AMPA Receptors Confers Live-Cell Fluorescent Biosensors. ACS Chem. Biol. 2018, 13 (7), 1880-1889.
53. Kajino, H.; Nagatani, T.; Oi, M.; Kujirai, T.; Kurumizaka, H.; Nishiyama, A.; Nakanishi, M.; Yamatsugu, K.; Kawashima, S. A.; Kanai, M. Synthetic Hyperacetylation of Nucleosomal Histones. RSC Chem Biol 2020, 1 (2), 56-59.
54. Barf, T.; Covey, T.; Izumi, R.; van de Kar, B.; Gulrajani, M.; van Lith, B.; van Hoek, M.; de Zwart, E.; Mittag, D.; Demont, D.; Verkaik, S.; Krantz, F.; Pearson, P. G.; Ulrich, R.; Kaptein, A. Acalabrutinib (ACP-196): A Covalent Bruton Tyrosine Kinase Inhibitor with a Differentiated Selectivity and In Vivo Potency Profile. J. Pharmacol. Exp. Ther. 2017, 363 (2), 240-252.
55. Hatai, J.; Prasad, P. K.; Lahav-Mankovski, N.; Oppenheimer-Low, N.; Unger, T.; Sirkis, Y. F.; Dadosh, T.; Motiei, L.; Margulies, D. Assessing Changes in the Expression Levels of Cell Surface Proteins with a Turn-on Fluorescent Molecular Probe. Chem. Commun. 2021, 57 (15), 1875-1878.
56. Unger-Angel, L.; Rout, B.; Ilani, T.; Eisenstein, M.; Motiei, L.; Margulies, D. Protein Recognition by Bivalent, “turn-On” fluorescent Molecular Probes. Chem. Sci. 2015, 6 (10), 5419-5425.

1. A Covalent Ligand Directed Releasing (CoLDR) Compound represented by the structure of formula I: 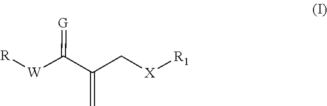
wherein:
R is a protein binding ligand, a fluorescent a chemiluminescent, a radiolabeled probe or a bio-active group;
R₁ is a releasing group comprising a protein binding ligand, a fluorescent, a chemiluminescent, a radiolabeled probe or a bio-active group;
wherein R and R₁ are different and at least one of R and R1 is a protein binding ligand;
W is a bond, NH, O, CH₂ or a linker;
G is O or S; and
X is a bond or a linker: wherein the linker comprises an alkyl, an aryl, an ester bond, an amide bond, a PEG, a carbamate bond, an anhydride bond, an oxygen atom, an amine, a sulfur atom, a nitrogen atom, a dendrimer, a self immolative linker or combination thereof;
wherein, if X is a bond then R₁ is linked to the backbone structure directly via an ester bond, an amide bond, an anhydride bond, a carbamate bond, an oxygen atom, a sulfur atom or a nitrogen atom: wherein the bio-active group is selected from the group consisting of a an approved drug, a targeted inhibitor, a cytotoxic, a chemotherapeutic, a radiopharmaceutical, substructure and derivative thereof; wherein the protein binding ligand is selected from the group consisting of acrylamide-based, vinylsulfone based, α,β unsaturated carbonyl based protein inhibitor and analog thereof.

2. The CoLDR Compound according to claim 1, wherein the CoLDR Compound is represented by the structure of formula IA: 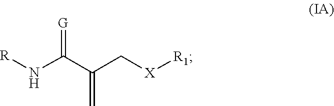
wherein R, R₁, G and X are as defined in claim 1.

3. The CoLDR Compound according to claim 1, wherein the CoLDR Compound is represented by the structure of formula IB:

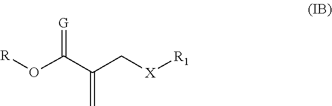

4. The CoLDR Compound according to claim 1, wherein the CoLDR Compound is represented by the structure of formula IC: 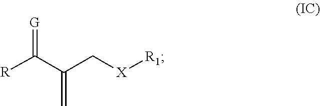
wherein R, R₁, G and X are as defined in claim 1.

5. (canceled)

6. (canceled)

7. The CoLDR Compound according to claim 1, wherein X is a bond.

8. The CoLDR Compound according to claim 1, wherein R is a protein binding ligand and R₁ is a fluorescent, a chemiluminescent or a radiolabeled probe.

9. The CoLDR Compound according to claim 1, wherein R is a protein binding ligand and R1 is a bio-active group.

10. The CoLDR Compound according to claim 1, wherein R is a fluorescent or chemiluminescent probe and R₁ is a protein binding ligand.

11. The CoLDR Compound according to claim 1, wherein R is a bio-active group and R₁ is a protein binding ligand.

12. The CoLDR Compound according to claim 1, wherein R or R₁ are both protein binding ligands and one of R or R¹ is a Ubiquitin ligase binder, thereby obtaining a CoLDR-based protein PROTAC compound.

13. (canceled)

14. The CoLDR Compound according to claim 1, wherein upon interaction between a protein and the protein binding ligand, R₁ (the Releasing Compound) is released.

15. The CoLDR Compound according to claim 1, wherein a covalent bond is formed between a protein and the protein binding ligand.

16. The CoLDR Compound according to claim 15, wherein the covalent bond is formed via a nucleophilic moiety of the protein being a thiol, an amine or a hydroxyl group and the double bond (—C═CH2) of the compounds of formula I, IA, IB or IC.

17. A prodrug comprising a Covalent Ligand Directed Releasing (CoLDR) Compound according to claim 1, wherein R is a protein binding ligand and R₁ is a drug or a targeted inhibitor, wherein, upon interaction between a protein and the protein binding ligand, the drug or the targeted inhibitor is released, wherein the CoLDR compound of the structure of Formula I, IA, IB or IC.

18. The prodrug according to claim 17, wherein a covalent bond is formed between the protein and the protein binding ligand.

19. The prodrug according to claim 17, wherein the covalent bond is formed via a nucleophilic moiety of the protein being a thiol, an amine or a hydroxyl group and the double bond (—C═CH₂) of the compounds of formula I, IA, IB or IC.

20. A pharmaceutical composition comprising the prodrug of claim 17, and a pharmaceutical acceptable carrier.

21. A protein sensor or a protein label comprising a Covalent Ligand Directed Releasing (CoLDR) Compound according to claim 1, wherein R or R₁ is a fluorescent probe or a chemiluminescent probe, wherein,
if R is a fluorescent probe or a chemiluminescent probe, and R₁ is a protein binding ligand; upon interaction between a protein and the protein bindingligand, the ligand is released and the fluorescent or the chemiluminescent probe is covalently attached to the protein and thereby results in change in fluorescence or chemiluminescence of the probe (FIG. 2); or
if R is a protein target ligand and R₁ is a fluorescent probe or a chemiluminescent probe, upon interaction between a protein and the protein binding ligand, the fluorescent probe or the or the chemiluminescent probe is released and the protein binding ligand is covalently attached to the protein and thereby results in change in fluorescence or chemiluminescence of the probes. (FIG. 1).

22. The protein sensor according to claim 21, wherein a covalent bond is formed between the protein and the protein binding ligand.

23. The protein sensor according to claim 22, wherein the covalent bond is formed via a nucleophilic group of the protein being a thiol, an amine or a hydroxyl group and the double bond (—C═CH₂) of the CoLDR Compound of formula I, IA, IB or IC.

24. A protein proximity inducer of a first protein and a second protein comprising a Covalent Ligand Directed Releasing (CoLDR) Compound according to claim 1, wherein R is a protein binding ligand for the first protein and R₁ is another protein binding ligand for the second protein, wherein, upon interaction between the second protein and the its protein binding ligand, R₁ is released, and the second protein is then active and is labeled with R, inducing a new interaction with the first protein.
